SEQUENCE LISTING (324 sequences)

SEQ ID NO: 1 - protein, 18 aa, h. sapiens
QQTDRSSPVRPSSEKNDC

SEQ ID NO: 2 - protein, 19 aa, h. sapiens
PKDMNNFKLERERSYELVR

SEQ ID NO: 3 - protein, 10 aa, h. sapiens
CTVKDAPSRK

SEQ ID NO: 4 - DNA, 36 bp, h. sapiens
gacgcaccaagccgaaagtaaaaacacaaaaacaga

SEQ ID NO: 5 - DNA, 35 bp, Artificial Sequence
  Description of Artificial Sequencepcr primer
gacgcaccaagccgaaagctcgagaagcttaacag

SEQ ID NO: 6 - protein, 9 aa, Artificial Sequence
  Description of Artificial Sequencepredicted peptide
RRIKGITKK

SEQ ID NO: 7 - protein, 9 aa, Artificial Sequence
  Description of Artificial Sequencepredicted peptide
KRYFELIHR

SEQ ID NO: 8 - protein, 9 aa, Artificial Sequence
  Description of Artificial Sequencepredicted peptide
GRFTKPVLL

SEQ ID NO: 9 - protein, 9 aa, Artificial Sequence
  Description of Artificial Sequencepredicted peptide
ARWERVRRY

SEQ ID NO: 10 - protein, 9 aa, Artificial Sequence
  Description of Artificial Sequencepredicted peptide
FRLQPAGEA

SEQ ID NO: 11 - protein, 9 aa, Artificial Sequence
  Description of Artificial Sequencepredicted peptide
SRVDASDDN

SEQ ID NO: 12 - protein, 9 aa, Artificial Sequence
  Description of Artificial Sequencepredicted peptide
SRSNITEQN

SEQ ID NO: 13 - protein, 9 aa, Artificial Sequence
  Description of Artificial Sequencepredicted peptide
SRASVTEAN

SEQ ID NO: 14 - protein, 9 aa, Artificial Sequence
  Description of Artificial Sequencepredicted peptide
SRALVTEVN

SEQ ID NO: 15 - protein, 9 aa, Artificial Sequence
  Description of Artificial Sequencepredicted peptide
SRAWVTEGN

SEQ ID NO: 16 - protein, 9 aa, Artificial Sequence
  Description of Artificial Sequencepredicted peptide
SRAIVTETN

SEQ ID NO: 17 - protein, 9 aa, Artificial Sequence
  Description of Artificial Sequencepredicted peptide
RRMGCPTPK

SEQ ID NO: 18 - protein, 9 aa, Artificial Sequence
  Description of Artificial Sequencepredicted peptide
TRYRSGGGK

SEQ ID NO: 19 - protein, 9 aa, Artificial Sequence
  Description of Artificial Sequencepredicted peptide
RRAKRMRAE

SEQ ID NO: 20 - protein, 9 aa, Artificial Sequence
  Description of Artificial Sequencepredicted peptide
ARTHNLRRR

SEQ ID NO: 21 - protein, 9 aa, Artificial Sequence
  Description of Artificial Sequencepredicted peptide
SRSKKATNA

SEQ ID NO: 22 - protein, 9 aa, Artificial Sequence
  Description of Artificial Sequencepredicted peptide
RRLEREGLS

SEQ ID NO: 23 - protein, 9 aa, Artificial Sequence
  Description of Artificial Sequencepredicted peptide
RRSEKPPLE

SEQ ID NO: 24 - protein, 9 aa, Artificial Sequence
  Description of Artificial Sequencepredicted peptide
MRIRLEQAR

SEQ ID NO: 25 - protein, 9 aa, Artificial Sequence
  Description of Artificial Sequencepredicted peptide
PRIQRLNEL

SEQ ID NO: 26 - protein, 9 aa, Artificial Sequence
  Description of Artificial Sequencepredicted peptide
MRAEAMNIK

SEQ ID NO: 27 - protein, 9 aa, Artificial Sequence
  Description of Artificial Sequencepredicted peptide
VRKLGVIDS

SEQ ID NO: 28 - protein, 9 aa, Artificial Sequence
  Description of Artificial Sequencepredicted peptide
ARWLEEVQQ

SEQ ID NO: 29 - protein, 9 aa, Artificial Sequence
  Description of Artificial Sequencepredicted peptide
YRYTLDDLY

SEQ ID NO: 30 - protein, 9 aa, Artificial Sequence
  Description of Artificial Sequencepredicted peptide
GRVPVLDTL

SEQ ID NO: 31 - protein, 9 aa, Artificial Sequence
  Description of Artificial Sequencepredicted peptide
ERSYELVRS

SEQ ID NO: 32 - protein, 9 aa, Artificial Sequence
  Description of Artificial Sequencepredicted peptide
QRLNELEAQ

SEQ ID NO: 33 - protein, 9 aa, Artificial Sequence
  Description of Artificial Sequencepredicted peptide
RRRMGCPTP

SEQ ID NO: 34 - protein, 9 aa, Artificial Sequence
  Description of Artificial Sequencepredicted peptide
ERKDYIVEN

SEQ ID NO: 35 - protein, 9 aa, Artificial Sequence
  Description of Artificial Sequencepredicted peptide
GRSIPVHLN

SEQ ID NO: 36 - protein, 9 aa, Artificial Sequence
  Description of Artificial Sequencepredicted peptide
LRYMIERTV

SEQ ID NO: 37 - protein, 9 aa, Artificial Sequence
  Description of Artificial Sequencepredicted peptide
ERVKKMRTP

SEQ ID NO: 38 - protein, 9 aa, Artificial Sequence
  Description of Artificial Sequencepredicted peptide
ERILNPYNL

SEQ ID NO: 39 - protein, 9 aa, Artificial Sequence
  Description of Artificial Sequencepredicted peptide
GRQCVEHYR

SEQ ID NO: 40 - protein, 9 aa, Artificial Sequence
  Description of Artificial Sequencepredicted peptide
ARLQELLTV

SEQ ID NO: 41 - protein, 9 aa, Artificial Sequence
  Description of Artificial Sequencepredicted peptide
PRLETLVAE

SEQ ID NO: 42 - protein, 9 aa, Artificial Sequence
  Description of Artificial Sequencepredicted peptide
ARLREMEAL

SEQ ID NO: 43 - protein, 9 aa, Artificial Sequence
  Description of Artificial Sequencepredicted peptide
KRRLEREGL

SEQ ID NO: 44 - protein, 9 aa, Artificial Sequence
  Description of Artificial Sequencepredicted peptide
WRLVSTIEE

SEQ ID NO: 45 - protein, 9 aa, Artificial Sequence
  Description of Artificial Sequencepredicted peptide
LRHLRLVTQ

SEQ ID NO: 46 - protein, 9 aa, Artificial Sequence
  Description of Artificial Sequencepredicted peptide
IRLEQARWL

SEQ ID NO: 47 - protein, 9 aa, Artificial Sequence
  Description of Artificial Sequencepredicted peptide
LRLANEGKL

SEQ ID NO: 48 - protein, 9 aa, Artificial Sequence
  Description of Artificial Sequencepredicted peptide
VRLPEGDAL

SEQ ID NO: 49 - protein, 9 aa, Artificial Sequence
  Description of Artificial Sequencepredicted peptide
ERTVNWQHR

SEQ ID NO: 50 - protein, 9 aa, Artificial Sequence
  Description of Artificial Sequencepredicted peptide
DRSSPVRPS

SEQ ID NO: 51 - protein, 9 aa, Artificial Sequence
  Description of Artificial Sequencepredicted peptide
CRGKRDGIN

SEQ ID NO: 52 - protein, 9 aa, Artificial Sequence
  Description of Artificial Sequencepredicted peptide
EREGLSSER

SEQ ID NO: 53 - protein, 9 aa, Artificial Sequence
  Description of Artificial Sequencepredicted peptide
ERWERVKKM

SEQ ID NO: 54 - protein, 9 aa, Artificial Sequence
  Description of Artificial Sequencepredicted peptide
ARDYTLRTF

SEQ ID NO: 55 - protein, 9 aa, Artificial Sequence
  Description of Artificial Sequencepredicted peptide
PRAYHSGFN

SEQ ID NO: 56 - protein, 9 aa, Artificial Sequence
  Description of Artificial Sequencepredicted peptide
HRYCVFSHD

SEQ ID NO: 57 - protein, 9 aa, Artificial Sequence
  Description of Artificial Sequencepredicted peptide
LRIWLCPHC

SEQ ID NO: 58 - protein, 9 aa, Artificial Sequence
  Description of Artificial Sequencepredicted peptide
SRWQASAGQ

SEQ ID NO: 59 - protein, 9 aa, Artificial Sequence
  Description of Artificial Sequencepredicted peptide
GRSCIPLHG

SEQ ID NO: 60 - protein, 9 aa, Artificial Sequence
  Description of Artificial Sequencepredicted peptide
ERKLKRRLE

SEQ ID NO: 61 - protein, 9 aa, Artificial Sequence
  Description of Artificial Sequencepredicted peptide
DRKWTKIAT

SEQ ID NO: 62 - protein, 9 aa, Artificial Sequence
  Description of Artificial Sequencepredicted peptide
VRDGKIKLS

SEQ ID NO: 63 - protein, 9 aa, Artificial Sequence
  Description of Artificial Sequencepredicted peptide
LRYRYTLDD

SEQ ID NO: 64 - protein, 9 aa, Artificial Sequence
  Description of Artificial Sequencepredicted peptide
KRQTRYRSG

SEQ ID NO: 65 - protein, 9 aa, Artificial Sequence
  Description of Artificial Sequencepredicted peptide
LRQFVTQLY

SEQ ID NO: 66 - protein, 9 aa, Artificial Sequence
  Description of Artificial Sequencepredicted peptide
VRSAETHSL

SEQ ID NO: 67 - protein, 9 aa, Artificial Sequence
  Description of Artificial Sequencepredicted peptide
TRVKLNFLD

SEQ ID NO: 68 - protein, 9 aa, Artificial Sequence
  Description of Artificial Sequencepredicted peptide
LRTFGEMAD

SEQ ID NO: 69 - protein, 9 aa, Artificial Sequence
  Description of Artificial Sequencepredicted peptide
QRIRVRLPE

SEQ ID NO: 70 - protein, 9 aa, Artificial Sequence
  Description of Artificial Sequencepredicted peptide
IRVRLPEGD

SEQ ID NO: 71 - protein, 9 aa, Artificial Sequence
  Description of Artificial Sequencepredicted peptide
IRGHYERIL

SEQ ID NO: 72 - protein, 9 aa, Artificial Sequence
  Description of Artificial Sequencepredicted peptide
ARRAKRMRA

SEQ ID NO: 73 - protein, 9 aa, Artificial Sequence
  Description of Artificial Sequencepredicted peptide
DRLLLCDGC

SEQ ID NO: 74 - protein, 9 aa, Artificial Sequence
  Description of Artificial Sequencepredicted peptide
YRSGGGKSQ

SEQ ID NO: 75 - protein, 9 aa, Artificial Sequence
  Description of Artificial Sequencepredicted peptide
KLYDIVQYV

SEQ ID NO: 76 - protein, 9 aa, Artificial Sequence
  Description of Artificial Sequencepredicted peptide
YIWETCTPL

SEQ ID NO: 77 - protein, 9 aa, Artificial Sequence
  Description of Artificial Sequencepredicted peptide
TMMTVQARI

SEQ ID NO: 78 - protein, 9 aa, Artificial Sequence
  Description of Artificial Sequencepredicted peptide
TTATHQARA

SEQ ID NO: 79 - protein, 9 aa, Artificial Sequence
  Description of Artificial Sequencepredicted peptide
TVATWQARC

SEQ ID NO: 80 - protein, 9 aa, Artificial Sequence
  Description of Artificial Sequencepredicted peptide
TVATYQART

SEQ ID NO: 81 - protein, 9 aa, Artificial Sequence
  Description of Artificial Sequencepredicted peptide
DVRTRHRVT

SEQ ID NO: 82 - protein, 9 aa, Artificial Sequence
  Description of Artificial Sequencepredicted peptide
DVRTNDDFT

SEQ ID NO: 83 - protein, 9 aa, Artificial Sequence
  Description of Artificial Sequencepredicted peptide
DVRTDDDFT

SEQ ID NO: 84 - protein, 9 aa, Artificial Sequence
  Description of Artificial Sequencepredicted peptide
TLMTHEVPV

SEQ ID NO: 85 - protein, 9 aa, Artificial Sequence
  Description of Artificial Sequencepredicted peptide
LLKDLLNRV

SEQ ID NO: 86 - protein, 9 aa, Artificial Sequence
  Description of Artificial Sequencepredicted peptide
KLLSPLQDV

SEQ ID NO: 87 - protein, 9 aa, Artificial Sequence
  Description of Artificial Sequencepredicted peptide
RLETLVAEV

SEQ ID NO: 88 - protein, 9 aa, Artificial Sequence
  Description of Artificial Sequencepredicted peptide
ALKDSVQRA

SEQ ID NO: 89 - protein, 9 aa, Artificial Sequence
  Description of Artificial Sequencepredicted peptide
WLPLGRQCV

SEQ ID NO: 90 - protein, 9 aa, Artificial Sequence
  Description of Artificial Sequencepredicted peptide
RLIDLGVGL

SEQ ID NO: 91 - protein, 9 aa, Artificial Sequence
  Description of Artificial Sequencepredicted peptide
FLPPPECPV

SEQ ID NO: 92 - protein, 9 aa, Artificial Sequence
  Description of Artificial Sequencepredicted peptide
QLAEMRIRL

SEQ ID NO: 93 - protein, 9 aa, Artificial Sequence
  Description of Artificial Sequencepredicted peptide
WLEEVQQAC

SEQ ID NO: 94 - protein, 9 aa, Artificial Sequence
  Description of Artificial Sequencepredicted peptide
SLNSLATAV

SEQ ID NO: 95 - protein, 9 aa, Artificial Sequence
  Description of Artificial Sequencepredicted peptide
VLDTLIELV

SEQ ID NO: 96 - protein, 9 aa, Artificial Sequence
  Description of Artificial Sequencepredicted peptide
SLPRLETLV

SEQ ID NO: 97 - protein, 9 aa, Artificial Sequence
  Description of Artificial Sequencepredicted peptide
NLKFVQDRV

SEQ ID NO: 98 - protein, 9 aa, Artificial Sequence
  Description of Artificial Sequencepredicted peptide
PLHGVSPEV

SEQ ID NO: 99 - protein, 9 aa, Artificial Sequence
  Description of Artificial Sequencepredicted peptide
EIEEIPAYL

SEQ ID NO: 100 - protein, 9 aa, Artificial Sequence
  Description of Artificial Sequencepredicted peptide
CLIPPLHDV

SEQ ID NO: 101 - protein, 9 aa, Artificial Sequence
  Description of Artificial Sequencepredicted peptide
GLLVCLHHV

SEQ ID NO: 102 - protein, 9 aa, Artificial Sequence
  Description of Artificial Sequencepredicted peptide
TLDDMRRLI

SEQ ID NO: 103 - protein, 9 aa, Artificial Sequence
  Description of Artificial Sequencepredicted peptide
SLSDLERAL

SEQ ID NO: 104 - protein, 9 aa, Artificial Sequence
  Description of Artificial Sequencepredicted peptide
SLQRIRVRL

SEQ ID NO: 105 - protein, 9 aa, Artificial Sequence
  Description of Artificial Sequencepredicted peptide
ELLMEAQLL

SEQ ID NO: 106 - protein, 9 aa, Artificial Sequence
  Description of Artificial Sequencepredicted peptide
QLENVMKKL

SEQ ID NO: 107 - protein, 9 aa, Artificial Sequence
  Description of Artificial Sequencepredicted peptide
ELFVSQPDL

SEQ ID NO: 108 - protein, 9 aa, Artificial Sequence
  Description of Artificial Sequencepredicted peptide
KINKKKSLV

SEQ ID NO: 109 - protein, 9 aa, Artificial Sequence
  Description of Artificial Sequencepredicted peptide
SLATAVKEI

SEQ ID NO: 110 - protein, 9 aa, Artificial Sequence
  Description of Artificial Sequencepredicted peptide
PLQDVDIKI

SEQ ID NO: 111 - protein, 9 aa, Artificial Sequence
  Description of Artificial Sequencepredicted peptide
PLEKILPLL

SEQ ID NO: 112 - protein, 9 aa, Artificial Sequence
  Description of Artificial Sequencepredicted peptide
VLAHITADI

SEQ ID NO: 113 - protein, 9 aa, Artificial Sequence
  Description of Artificial Sequencepredicted peptide
ALRETVRKL

SEQ ID NO: 114 - protein, 9 aa, Artificial Sequence
  Description of Artificial Sequencepredicted peptide
ELRQFVTQL

SEQ ID NO: 115 - protein, 9 aa, Artificial Sequence
  Description of Artificial Sequencepredicted peptide
RLANEGKLL

SEQ ID NO: 116 - protein, 9 aa, Artificial Sequence
  Description of Artificial Sequencepredicted peptide
PLLASLQRI

SEQ ID NO: 117 - protein, 9 aa, Artificial Sequence
  Description of Artificial Sequencepredicted peptide
EIQELYQTL

SEQ ID NO: 118 - protein, 9 aa, Artificial Sequence
  Description of Artificial Sequencepredicted peptide
ILNPYNLFL

SEQ ID NO: 119 - protein, 9 aa, Artificial Sequence
  Description of Artificial Sequencepredicted peptide
VLDVVVAST

SEQ ID NO: 120 - protein, 9 aa, Artificial Sequence
  Description of Artificial Sequencepredicted peptide
TVNELRQFV

SEQ ID NO: 121 - protein, 9 aa, Artificial Sequence
  Description of Artificial Sequencepredicted peptide
CLDPSSLTL

SEQ ID NO: 122 - protein, 9 aa, Artificial Sequence
  Description of Artificial Sequencepredicted peptide
KILPLLASL

SEQ ID NO: 123 - protein, 9 aa, Artificial Sequence
  Description of Artificial Sequencepredicted peptide
TLLAKPSPA

SEQ ID NO: 124 - protein, 9 aa, Artificial Sequence
  Description of Artificial Sequencepredicted peptide
CLQPEGDEV

SEQ ID NO: 125 - protein, 9 aa, Artificial Sequence
  Description of Artificial Sequencepredicted peptide
RLNELEAQT

SEQ ID NO: 126 - protein, 9 aa, Artificial Sequence
  Description of Artificial Sequencepredicted peptide
KLNFLDQIA

SEQ ID NO: 127 - protein, 9 aa, Artificial Sequence
  Description of Artificial Sequencepredicted peptide
NMPVMEQSV

SEQ ID NO: 128 - protein, 9 aa, Artificial Sequence
  Description of Artificial Sequencepredicted peptide
GMKLPWLYV

SEQ ID NO: 129 - protein, 9 aa, Artificial Sequence
  Description of Artificial Sequencepredicted peptide
QLYALPCVL

SEQ ID NO: 130 - protein, 9 aa, Artificial Sequence
  Description of Artificial Sequencepredicted peptide
GLKRKQRKL

SEQ ID NO: 131 - protein, 9 aa, Artificial Sequence
  Description of Artificial Sequencepredicted peptide
DLFQLNKLV

SEQ ID NO: 132 - protein, 9 aa, Artificial Sequence
  Description of Artificial Sequencepredicted peptide
YLHWGEPKT

SEQ ID NO: 133 - protein, 9 aa, Artificial Sequence
  Description of Artificial Sequencepredicted peptide
SLVSFKALI

SEQ ID NO: 134 - protein, 9 aa, Artificial Sequence
  Description of Artificial Sequencepredicted peptide
LLSEETPSA

SEQ ID NO: 135 - protein, 9 aa, Artificial Sequence
  Description of Artificial Sequencepredicted peptide
PVLDTLIEL

SEQ ID NO: 136 - protein, 9 aa, Artificial Sequence
  Description of Artificial Sequencepredicted peptide
LLEVLCPRC

SEQ ID NO: 137 - protein, 9 aa, Artificial Sequence
  Description of Artificial Sequencepredicted peptide
ALTESKETA

SEQ ID NO: 138 - protein, 9 aa, Artificial Sequence
  Description of Artificial Sequencepredicted peptide
TLHPGPRPA

SEQ ID NO: 139 - protein, 9 aa, Artificial Sequence
  Description of Artificial Sequencepredicted peptide
ELQGSTLKI

SEQ ID NO: 140 - protein, 9 aa, Artificial Sequence
  Description of Artificial Sequencepredicted peptide
NLFLSGDSL

SEQ ID NO: 141 - protein, 9 aa, Artificial Sequence
  Description of Artificial Sequencepredicted peptide
TLRTFGEMA

SEQ ID NO: 142 - protein, 9 aa, Artificial Sequence
  Description of Artificial Sequencepredicted peptide
LVSTIEEDV

SEQ ID NO: 143 - protein, 9 aa, Artificial Sequence
  Description of Artificial Sequencepredicted peptide
CLHHVKELC

SEQ ID NO: 144 - protein, 9 aa, Artificial Sequence
  Description of Artificial Sequencepredicted peptide
DLYPMMNAL

SEQ ID NO: 145 - protein, 9 aa, Artificial Sequence
  Description of Artificial Sequencepredicted peptide
DLLRHLRLV

SEQ ID NO: 146 - protein, 9 aa, Artificial Sequence
  Description of Artificial Sequencepredicted peptide
ELEAQTRVK

SEQ ID NO: 147 - protein, 9 aa, Artificial Sequence
  Description of Artificial Sequencepredicted peptide
TLKIPHVER

SEQ ID NO: 148 - protein, 9 aa, Artificial Sequence
  Description of Artificial Sequencepredicted peptide
ILDLFQLNK

SEQ ID NO: 149 - protein, 9 aa, Artificial Sequence
  Description of Artificial Sequencepredicted peptide
KLVAEEGGF

SEQ ID NO: 150 - protein, 9 aa, Artificial Sequence
  Description of Artificial Sequencepredicted peptide
AVGSHIRGH

SEQ ID NO: 151 - protein, 9 aa, Artificial Sequence
  Description of Artificial Sequencepredicted peptide
NLTTDTKDK

SEQ ID NO: 152 - protein, 9 aa, Artificial Sequence
  Description of Artificial Sequencepredicted peptide
MVPTELVEK

SEQ ID NO: 153 - protein, 9 aa, Artificial Sequence
  Description of Artificial Sequencepredicted peptide
ELVEKEFWR

SEQ ID NO: 154 - protein, 9 aa, Artificial Sequence
  Description of Artificial Sequencepredicted peptide
KLSPEEEEY

SEQ ID NO: 155 - protein, 9 aa, Artificial Sequence
  Description of Artificial Sequencepredicted peptide
VMEQSVLAH

SEQ ID NO: 156 - protein, 9 aa, Artificial Sequence
  Description of Artificial Sequencepredicted peptide
YVGMCFSSF

SEQ ID NO: 157 - protein, 9 aa, Artificial Sequence
  Description of Artificial Sequencepredicted peptide
FVSQPDLLH

SEQ ID NO: 158 - protein, 9 aa, Artificial Sequence
  Description of Artificial Sequencepredicted peptide
LMTHEVPVY

SEQ ID NO: 159 - protein, 9 aa, Artificial Sequence
  Description of Artificial Sequencepredicted peptide
FVITFPRAY

SEQ ID NO: 160 - protein, 9 aa, Artificial Sequence
  Description of Artificial Sequencepredicted peptide
TVDWLPLGR

SEQ ID NO: 161 - protein, 9 aa, Artificial Sequence
  Description of Artificial Sequencepredicted peptide
PLGRQCVEH

SEQ ID NO: 162 - protein, 9 aa, Artificial Sequence
  Description of Artificial Sequencepredicted peptide
CVEHYRLLH

SEQ ID NO: 163 - protein, 9 aa, Artificial Sequence
  Description of Artificial Sequencepredicted peptide
RLLHRYCVF

SEQ ID NO: 164 - protein, 8 aa, Artificial Sequence
  Description of Artificial Sequencepredicted peptide
EICKMASK

SEQ ID NO: 165 - protein, 9 aa, Artificial Sequence
  Description of Artificial Sequencepredicted peptide
VVVASTVQK

SEQ ID NO: 166 - protein, 9 aa, Artificial Sequence
  Description of Artificial Sequencepredicted peptide
KLGVIDSER

SEQ ID NO: 167 - protein, 8 aa, Artificial Sequence
  Description of Artificial Sequencepredicted peptide
CVKCTTCF

SEQ ID NO: 168 - protein, 9 aa, Artificial Sequence
  Description of Artificial Sequencepredicted peptide
LLVCLHHVK

SEQ ID NO: 169 - protein, 9 aa, Artificial Sequence
  Description of Artificial Sequencepredicted peptide
ELCSCPPYK

SEQ ID NO: 170 - protein, 9 aa, Artificial Sequence
  Description of Artificial Sequencepredicted peptide
PMMNALKLR

SEQ ID NO: 171 - protein, 9 aa, Artificial Sequence
  Description of Artificial Sequencepredicted peptide
ALKLRAESY

SEQ ID NO: 172 - protein, 9 aa, Artificial Sequence
  Description of Artificial Sequencepredicted peptide
NVNEALEAK

SEQ ID NO: 173 - protein, 9 aa, Artificial Sequence
  Description of Artificial Sequencepredicted peptide
ALEAKINKK

SEQ ID NO: 174 - protein, 9 aa, Artificial Sequence
  Description of Artificial Sequencepredicted peptide
ALIEESEMK

SEQ ID NO: 175 - protein, 9 aa, Artificial Sequence
  Description of Artificial Sequencepredicted peptide
RLVTQDAEK

SEQ ID NO: 176 - protein, 9 aa, Artificial Sequence
  Description of Artificial Sequencepredicted peptide
LLNGKRQTR

SEQ ID NO: 177 - protein, 9 aa, Artificial Sequence
  Description of Artificial Sequencepredicted peptide
VLSQTPLLK

SEQ ID NO: 178 - protein, 9 aa, Artificial Sequence
  Description of Artificial Sequencepredicted peptide
PLLKDLLNR

SEQ ID NO: 179 - protein, 9 aa, Artificial Sequence
  Description of Artificial Sequencepredicted peptide
DLLNRVEDF

SEQ ID NO: 180 - protein, 9 aa, Artificial Sequence
  Description of Artificial Sequencepredicted peptide
DLLDVSFEF

SEQ ID NO: 181 - protein, 9 aa, Artificial Sequence
  Description of Artificial Sequencepredicted peptide
ELPQLAEMR

SEQ ID NO: 182 - protein, 9 aa, Artificial Sequence
  Description of Artificial Sequencepredicted peptide
SLTLDDMRR

SEQ ID NO: 183 - protein, 9 aa, Artificial Sequence
  Description of Artificial Sequencepredicted peptide
DLGVGLAPY

SEQ ID NO: 184 - protein, 9 aa, Artificial Sequence
  Description of Artificial Sequencepredicted peptide
TVSEHWDDK

SEQ ID NO: 185 - protein, 9 aa, Artificial Sequence
  Description of Artificial Sequencepredicted peptide
SLLKARPRH

SEQ ID NO: 186 - protein, 9 aa, Artificial Sequence
  Description of Artificial Sequencepredicted peptide
YLPNGAALK

SEQ ID NO: 187 - protein, 9 aa, Artificial Sequence
  Description of Artificial Sequencepredicted peptide
PVHLNSLPR

SEQ ID NO: 188 - protein, 9 aa, Artificial Sequence
  Description of Artificial Sequencepredicted peptide
LVAEVQAWK

SEQ ID NO: 189 - protein, 9 aa, Artificial Sequence
  Description of Artificial Sequencepredicted peptide
SLLEVLCPR

SEQ ID NO: 190 - protein, 9 aa, Artificial Sequence
  Description of Artificial Sequencepredicted peptide
LLGLKRKQR

SEQ ID NO: 191 - protein, 9 aa, Artificial Sequence
  Description of Artificial Sequencepredicted peptide
AMATLGEAR

SEQ ID NO: 192 - protein, 9 aa, Artificial Sequence
  Description of Artificial Sequencepredicted peptide
EMEALQSLR

SEQ ID NO: 193 - protein, 9 aa, Artificial Sequence
  Description of Artificial Sequencepredicted peptide
SLRLANEGK

SEQ ID NO: 194 - protein, 9 aa, Artificial Sequence
  Description of Artificial Sequencepredicted peptide
PMIQCELCR

SEQ ID NO: 195 - protein, 9 aa, Artificial Sequence
  Description of Artificial Sequencepredicted peptide
GLRIWLCPH

SEQ ID NO: 196 - protein, 9 aa, Artificial Sequence
  Description of Artificial Sequencepredicted peptide
LLASLQRIR

SEQ ID NO: 197 - protein, 9 aa, Artificial Sequence
  Description of Artificial Sequencepredicted peptide
RLPEGDALR

SEQ ID NO: 198 - protein, 9 aa, Artificial Sequence
  Description of Artificial Sequencepredicted peptide
QLLSSGNLK

SEQ ID NO: 199 - protein, 9 aa, Artificial Sequence
  Description of Artificial Sequencepredicted peptide
LLSSGNLKF

SEQ ID NO: 200 - protein, 9 aa, Artificial Sequence
  Description of Artificial Sequencepredicted peptide
SLPDDWDNR

SEQ ID NO: 201 - protein, 9 aa, Artificial Sequence
  Description of Artificial Sequencepredicted peptide
SLPEIQELY

SEQ ID NO: 202 - protein, 9 aa, Artificial Sequence
  Description of Artificial Sequencepredicted peptide
ELYQTLLAK

SEQ ID NO: 203 - protein, 9 aa, Artificial Sequence
  Description of Artificial Sequencepredicted peptide
SLERKLKRR

SEQ ID NO: 204 - protein, 9 aa, Artificial Sequence
  Description of Artificial Sequencepredicted peptide
GLSSERWER

SEQ ID NO: 205 - protein, 9 aa, Artificial Sequence
  Description of Artificial Sequencepredicted peptide
RVKKMRTPK

SEQ ID NO: 206 - protein, 9 aa, Artificial Sequence
  Description of Artificial Sequencepredicted peptide
KMRTPKKKK

SEQ ID NO: 207 - protein, 9 aa, Artificial Sequence
  Description of Artificial Sequencepredicted peptide
DMNNFKLER

SEQ ID NO: 208 - protein, 9 aa, Artificial Sequence
  Description of Artificial Sequencepredicted peptide
ELVRSAETH

SEQ ID NO: 209 - protein, 9 aa, Artificial Sequence
  Description of Artificial Sequencepredicted peptide
GVSPEMAEK

SEQ ID NO: 210 - protein, 9 aa, Artificial Sequence
  Description of Artificial Sequencepredicted peptide
TVKDAPSRK

SEQ ID NO: 211 - protein, 9 aa, Artificial Sequence
  Description of Artificial Sequencepredicted peptide
ATTLHPGPR

SEQ ID NO: 212 - protein, 9 aa, Artificial Sequence
  Description of Artificial Sequencepredicted peptide
VFEPSWEEF

SEQ ID NO: 213 - protein, 9 aa, Artificial Sequence
  Description of Artificial Sequencepredicted peptide
FADPFAFIH

SEQ ID NO: 214 - protein, 9 aa, Artificial Sequence
  Description of Artificial Sequencepredicted peptide
PFAFIHKIR

SEQ ID NO: 215 - protein, 9 aa, Artificial Sequence
  Description of Artificial Sequencepredicted peptide
IAEQTGICK

SEQ ID NO: 216 - protein, 9 aa, Artificial Sequence
  Description of Artificial Sequencepredicted peptide
FACDVDKLH

SEQ ID NO: 217 - protein, 9 aa, Artificial Sequence
  Description of Artificial Sequencepredicted peptide
NFLDQIAKY

SEQ ID NO: 218 - protein, 9 aa, Artificial Sequence
  Description of Artificial Sequencepredicted peptide
GFAVVCKDR

SEQ ID NO: 219 - protein, 9 aa, Artificial Sequence
  Description of Artificial Sequencepredicted peptide
FAVVCKDRK

SEQ ID NO: 220 - protein, 9 aa, Artificial Sequence
  Description of Artificial Sequencepredicted peptide
RILNPYNLF

SEQ ID NO: 221 - protein, 9 aa, Artificial Sequence
  Description of Artificial Sequencepredicted peptide
LFLSGDSLR

SEQ ID NO: 222 - protein, 9 aa, Artificial Sequence
  Description of Artificial Sequencepredicted peptide
TTDTKDKEY

SEQ ID NO: 223 - protein, 9 aa, Artificial Sequence
  Description of Artificial Sequencepredicted peptide
PSETCPPAR

SEQ ID NO: 224 - protein, 9 aa, Artificial Sequence
  Description of Artificial Sequencepredicted peptide
PARRAKRMR

SEQ ID NO: 225 - protein, 9 aa, Artificial Sequence
  Description of Artificial Sequencepredicted peptide
EARTHNLRR

SEQ ID NO: 226 - protein, 9 aa, Artificial Sequence
  Description of Artificial Sequencepredicted peptide
PTPKCENEK

SEQ ID NO: 227 - protein, 9 aa, Artificial Sequence
  Description of Artificial Sequencepredicted peptide
SIKQEPIER

SEQ ID NO: 228 - protein, 9 aa, Artificial Sequence
  Description of Artificial Sequencepredicted peptide
YIVENEKEK

SEQ ID NO: 229 - protein, 9 aa, Artificial Sequence
  Description of Artificial Sequencepredicted peptide
KATNAVDLY

SEQ ID NO: 230 - protein, 9 aa, Artificial Sequence
  Description of Artificial Sequencepredicted peptide
TFCLIPPLH

SEQ ID NO: 231 - protein, 9 aa, Artificial Sequence
  Description of Artificial Sequencepredicted peptide
AFGFEQAAR

SEQ ID NO: 232 - protein, 9 aa, Artificial Sequence
  Description of Artificial Sequencepredicted peptide
GFEQAARDY

SEQ ID NO: 233 - protein, 9 aa, Artificial Sequence
  Description of Artificial Sequencepredicted peptide
QAARDYTLR

SEQ ID NO: 234 - protein, 9 aa, Artificial Sequence
  Description of Artificial Sequencepredicted peptide
TFGEMADAF

SEQ ID NO: 235 - protein, 9 aa, Artificial Sequence
  Description of Artificial Sequencepredicted peptide
MADAFKSDY

SEQ ID NO: 236 - protein, 9 aa, Artificial Sequence
  Description of Artificial Sequencepredicted peptide
PTELVEKEF

SEQ ID NO: 237 - protein, 9 aa, Artificial Sequence
  Description of Artificial Sequencepredicted peptide
GADIASKEF

SEQ ID NO: 238 - protein, 9 aa, Artificial Sequence
  Description of Artificial Sequencepredicted peptide
ASKEFGSGF

SEQ ID NO: 239 - protein, 9 aa, Artificial Sequence
  Description of Artificial Sequencepredicted peptide
EFGSGFPVR

SEQ ID NO: 240 - protein, 9 aa, Artificial Sequence
  Description of Artificial Sequencepredicted peptide
ATADICGMK

SEQ ID NO: 241 - protein, 9 aa, Artificial Sequence
  Description of Artificial Sequencepredicted peptide
SFCWHIEDH

SEQ ID NO: 242 - protein, 9 aa, Artificial Sequence
  Description of Artificial Sequencepredicted peptide
WSYSINYLH

SEQ ID NO: 243 - protein, 9 aa, Artificial Sequence
  Description of Artificial Sequencepredicted peptide
KTWYGVPGY

SEQ ID NO: 244 - protein, 9 aa, Artificial Sequence
  Description of Artificial Sequencepredicted peptide
MTHEVPVYR

SEQ ID NO: 245 - protein, 9 aa, Artificial Sequence
  Description of Artificial Sequencepredicted peptide
RTNQCAGEF

SEQ ID NO: 246 - protein, 9 aa, Artificial Sequence
  Description of Artificial Sequencepredicted peptide
CAGEFVITF

SEQ ID NO: 247 - protein, 9 aa, Artificial Sequence
  Description of Artificial Sequencepredicted peptide
VITFPRAYH

SEQ ID NO: 248 - protein, 9 aa, Artificial Sequence
  Description of Artificial Sequencepredicted peptide
FSHDEMICK

SEQ ID NO: 249 - protein, 9 aa, Artificial Sequence
  Description of Artificial Sequencepredicted peptide
MAIMIEDEK

SEQ ID NO: 250 - protein, 9 aa, Artificial Sequence
  Description of Artificial Sequencepredicted peptide
MIEDEKALR

SEQ ID NO: 251 - protein, 9 aa, Artificial Sequence
  Description of Artificial Sequencepredicted peptide
KALRETVRK

SEQ ID NO: 252 - protein, 9 aa, Artificial Sequence
  Description of Artificial Sequencepredicted peptide
VIDSERMDF

SEQ ID NO: 253 - protein, 9 aa, Artificial Sequence
  Description of Artificial Sequencepredicted peptide
MSAISCSCK

SEQ ID NO: 254 - protein, 9 aa, Artificial Sequence
  Description of Artificial Sequencepredicted peptide
CSCPPYKYK

SEQ ID NO: 255 - protein, 9 aa, Artificial Sequence
  Description of Artificial Sequencepredicted peptide
EALEAKINK

SEQ ID NO: 256 - protein, 9 aa, Artificial Sequence
  Description of Artificial Sequencepredicted peptide
LIEESEMKK

SEQ ID NO: 257 - protein, 9 aa, Artificial Sequence
  Description of Artificial Sequencepredicted peptide
KFPDNDLLR

SEQ ID NO: 258 - protein, 9 aa, Artificial Sequence
  Description of Artificial Sequencepredicted peptide
VAQQLLNGK

SEQ ID NO: 259 - protein, 9 aa, Artificial Sequence
  Description of Artificial Sequencepredicted peptide
LTVNELRQF

SEQ ID NO: 260 - protein, 9 aa, Artificial Sequence
  Description of Artificial Sequencepredicted peptide
SSLTLDDMR

SEQ ID NO: 261 - protein, 9 aa, Artificial Sequence
  Description of Artificial Sequencepredicted peptide
LAPYSAVEK

SEQ ID NO: 262 - protein, 9 aa, Artificial Sequence
  Description of Artificial Sequencepredicted peptide
SAVEKAMAR

SEQ ID NO: 263 - protein, 9 aa, Artificial Sequence
  Description of Artificial Sequencepredicted peptide
KAKSLLKAR

SEQ ID NO: 264 - protein, 9 aa, Artificial Sequence
  Description of Artificial Sequencepredicted peptide
KSLLKARPR

SEQ ID NO: 265 - protein, 9 aa, Artificial Sequence
  Description of Artificial Sequencepredicted peptide
NYLHWGEPK

SEQ ID NO: 266 - protein, 9 aa, Artificial Sequence
  Description of Artificial Sequencepredicted peptide
LVAEVQAWK

SEQ ID NO: 267 - protein, 9 aa, Artificial Sequence
  Description of Artificial Sequencepredicted peptide
ITADICGMK

SEQ ID NO: 268 - protein, 9 aa, Artificial Sequence
  Description of Artificial Sequencepredicted peptide
PPFACDVDK

SEQ ID NO: 269 - protein, 9 aa, Artificial Sequence
  Description of Artificial Sequencepredicted peptide
LNFLDQIAK

SEQ ID NO: 270 - protein, 9 aa, Artificial Sequence
  Description of Artificial Sequencepredicted peptide
WELQGSTLK

SEQ ID NO: 271 - protein, 9 aa, Artificial Sequence
  Description of Artificial Sequencepredicted peptide
LKIPHVERK

SEQ ID NO: 272 - protein, 9 aa, Artificial Sequence
  Description of Artificial Sequencepredicted peptide
TKMGFAPGK

SEQ ID NO: 273 - protein, 9 aa, Artificial Sequence
  Description of Artificial Sequencepredicted peptide
MRAEAMNIK

SEQ ID NO: 274 - protein, 9 aa, Artificial Sequence
  Description of Artificial Sequencepredicted peptide
RRMGCPTPK

SEQ ID NO: 275 - protein, 9 aa, Artificial Sequence
  Description of Artificial Sequencepredicted peptide
KDYIVENEK

SEQ ID NO: 276 - protein, 9 aa, Artificial Sequence
  Description of Artificial Sequencepredicted peptide
YIVENEKEK

SEQ ID NO: 277 - protein, 9 aa, Artificial Sequence
  Description of Artificial Sequencepredicted peptide
KCLAQECSK

SEQ ID NO: 278 - protein, 9 aa, Artificial Sequence
  Description of Artificial Sequencepredicted peptide
MVPTELVEK

SEQ ID NO: 279 - protein, 9 aa, Artificial Sequence
  Description of Artificial Sequencepredicted peptide
EYGADIASK

SEQ ID NO: 280 - protein, 9 aa, Artificial Sequence
  Description of Artificial Sequencepredicted peptide
SGFPVRDGK

SEQ ID NO: 281 - protein, 9 aa, Artificial Sequence
  Description of Artificial Sequencepredicted peptide
EQLENVMKK

SEQ ID NO: 282 - protein, 9 aa, Artificial Sequence
  Description of Artificial Sequencepredicted peptide
EMICKMASK

SEQ ID NO: 283 - protein, 9 aa, Artificial Sequence
  Description of Artificial Sequencepredicted peptide
VVVASTVQK

SEQ ID NO: 284 - protein, 9 aa, Artificial Sequence
  Description of Artificial Sequencepredicted peptide
MAIMIEDEK

SEQ ID NO: 285 - protein, 9 aa, Artificial Sequence
  Description of Artificial Sequencepredicted peptide
KALRETVRK

SEQ ID NO: 286 - protein, 9 aa, Artificial Sequence
  Description of Artificial Sequencepredicted peptide
MSAISCSCK

SEQ ID NO: 287 - protein, 9 aa, Artificial Sequence
  Description of Artificial Sequencepredicted peptide
LYPMMNALK

SEQ ID NO: 288 - protein, 9 aa, Artificial Sequence
  Description of Artificial Sequencepredicted peptide
NVNEALEAK

SEQ ID NO: 289 - protein, 9 aa, Artificial Sequence
  Description of Artificial Sequencepredicted peptide
EALEAKINK

SEQ ID NO: 290 - protein, 9 aa, Artificial Sequence
  Description of Artificial Sequencepredicted peptide
LEAKINKKK

SEQ ID NO: 291 - protein, 9 aa, Artificial Sequence
  Description of Artificial Sequencepredicted peptide
ALIEESEMK

SEQ ID NO: 292 - protein, 9 aa, Artificial Sequence
  Description of Artificial Sequencepredicted peptide
LIEESEMKK

SEQ ID NO: 293 - protein, 9 aa, Artificial Sequence
  Description of Artificial Sequencepredicted peptide
TRYRSGGGK

SEQ ID NO: 294 - protein, 9 aa, Artificial Sequence
  Description of Artificial Sequencepredicted peptide
EDFQQHSQK

SEQ ID NO: 295 - protein, 9 aa, Artificial Sequence
  Description of Artificial Sequencepredicted peptide
TVSEHWDDK

SEQ ID NO: 296 - protein, 9 aa, Artificial Sequence
  Description of Artificial Sequencepredicted peptide
CDIGLLGLK

SEQ ID NO: 297 - protein, 9 aa, Artificial Sequence
  Description of Artificial Sequencepredicted peptide
IGLLGLKRK

SEQ ID NO: 298 - protein, 9 aa, Artificial Sequence
  Description of Artificial Sequencepredicted peptide
LGLKRKQRK

SEQ ID NO: 299 - protein, 9 aa, Artificial Sequence
  Description of Artificial Sequencepredicted peptide
EPLPNGKKK

SEQ ID NO: 300 - protein, 9 aa, Artificial Sequence
  Description of Artificial Sequencepredicted peptide
SPLQDVDIK

SEQ ID NO: 301 - protein, 9 aa, Artificial Sequence
  Description of Artificial Sequencepredicted peptide
DIKICLCQK

SEQ ID NO: 302 - protein, 9 aa, Artificial Sequence
  Description of Artificial Sequencepredicted peptide
QLLSSGNLK

SEQ ID NO: 303 - protein, 9 aa, Artificial Sequence
  Description of Artificial Sequencepredicted peptide
ELYQTLLAK

SEQ ID NO: 304 - protein, 9 aa, Artificial Sequence
  Description of Artificial Sequencepredicted peptide
DGINSLERK

SEQ ID NO: 305 - protein, 9 aa, Artificial Sequence
  Description of Artificial Sequencepredicted peptide
RVKKMRTPK

SEQ ID NO: 306 - protein, 9 aa, Artificial Sequence
  Description of Artificial Sequencepredicted peptide
KKMRTPKKK

SEQ ID NO: 307 - protein, 9 aa, Artificial Sequence
  Description of Artificial Sequencepredicted peptide
KKIKLSHPK

SEQ ID NO: 308 - protein, 9 aa, Artificial Sequence
  Description of Artificial Sequencepredicted peptide
GVSPEMAEK

SEQ ID NO: 309 - protein, 9 aa, Artificial Sequence
  Description of Artificial Sequencepredicted peptide
YICVRCTVK

SEQ ID NO: 310 - protein, 9 aa, Artificial Sequence
  Description of Artificial Sequencepredicted peptide
TVKDAPSRK

SEQ ID NO: 311 - protein, 9 aa, Artificial Sequence
  Description of Artificial Sequencepredicted peptide
PTPKCENEK

SEQ ID NO: 312 - protein, 9 aa, Artificial Sequence
  Description of Artificial Sequencepredicted peptide
RTPKKKKIK

SEQ ID NO: 313 - protein, 9 aa, Artificial Sequence
  Description of Artificial Sequencepredicted peptide
PFAFIHKIR

SEQ ID NO: 314 - protein, 9 aa, Artificial Sequence
  Description of Artificial Sequencepredicted peptide
GFAVVCKDR

SEQ ID NO: 315 - protein, 9 aa, Artificial Sequence
  Description of Artificial Sequencepredicted peptide
LFLSGDSLR

SEQ ID NO: 316 - protein, 9 aa, Artificial Sequence
  Description of Artificial Sequencepredicted peptide
DTLIELVTR

SEQ ID NO: 317 - protein, 9 aa, Artificial Sequence
  Description of Artificial Sequencepredicted peptide
ATLGEARLR

SEQ ID NO: 318 - protein, 9 aa, Artificial Sequence
  Description of Artificial Sequencepredicted peptide
ADPFAFIHK

SEQ ID NO: 319 - protein, 9 aa, Artificial Sequence
  Description of Artificial Sequencepredicted peptide
IAEQTGICK

SEQ ID NO: 320 - protein, 9 aa, Artificial Sequence
  Description of Artificial Sequencepredicted peptide
ELEAQTRVK

SEQ ID NO: 321 - protein, 9 aa, Artificial Sequence
  Description of Artificial Sequencepredicted peptide
ILDLFQLNK

SEQ ID NO: 322 - protein, 9 aa, Artificial Sequence
  Description of Artificial Sequencepredicted peptide
EGGFAVVCK

SEQ ID NO: 323 - DNA, 6393 bp, h. sapiens
gccgcgtctgcacgggtctcggaccgagcggagctcgcagcctcggtcccggagcccaccttcgcctcgcccttgcccagcctgcggtgatggaggcggccaccacactgcacccaggcccgcgcccggcgctgcccctcgggggcccgggcccgctgggcgagttcctgcctccacccgagtgcccggtcttcgaacccagctgggaagagttcgcggaccccttcgctttcatccacaagatccggcccatagccgagcagactggcatctgtaaggtgcggccgccgccggattggcagccaccatttgcatgtgatgttgataaacttcattttacgccacgtatccagagactgaatgaattggaggcccaaactcgtgtaaaattgaatttcttggaccagattgcaaagtactgggagttacagggaagtactctgaaaattccacatgtggagaggaagatcttggacttatttcagcttaataagttagttgcagaagaaggtggatttgcagttgtttgcaaggatagaaaatggaccaaaattgctaccaagatggggtttgctcctggcaaagcagtgggctcacatatcagagggcattatgaacgaattctcaacccctacaacttattcctgtccggagacagcctaaggtgtttgcagaagccaaacctgaccacagacactaaggacaaggagtacaaaccccatgatattccccagaggcagtctgtgcagccttcggaaacgtgccccccagcccgacgagcaaaacgcatgagagcagaggccatgaatattaaaatagaacccgaggagacaacagaagccagaactcataatctgagacgtcgaatgggttgtccaactccaaaatgtgaaaatgagaaagaaatgaagagtagcatcaagcaagaacctattgagaggaaagattatattgtagaaaatgagaaggaaaagcccaagagtcgatctaaaaaagccaccaatgctgtggacctgtatgtctgtcttttatgtggcagtggcaatgatgaagaccggctactgttgtgtgatggctgtgatgacagttaccataccttttgcttgatcccacctctccatgatgttcccaagggagactggaggtgtcctaagtgtttggctcaggaatgtagtaagccacaagaagcatttggctttgaacaagcagccagggactataccctccgtacttttggggaaatggcagatgcgttcaaatctgattacttcaacatgccagtccatatggtccccacagagcttgttgagaaagaattttggagactagtaagcactattgaggaggatgtcacagtggaatatggagctgacattgcctcaaaggaatttggcagtggctttcctgtccgagatgggaaaatcaaactctcacctgaggaagaggagtatcttgatagtggctggaatttgaacaacatgccagtgatggagcagtctgtccttgcacatattactgctgatatatgtggcatgaaacttccttggttgtatgtgggaatgtgcttttcttcattctgttggcacattgaagaccactggagctattcaattaactacttgcactggggtgagccaaaaacctggtatggagtcccagggtatgctgctgagcagctagaaaatgtaatgaagaaactagctccagaactctttgtgtcccagccggatctcctccatcagcttgtgaccatcatgaaccccaataccctgatgactcatgaagtgcctgtttaccgaactaatcagtgtgctggggagtttgtgattacatttccaagagcctaccacagtggttttaaccagggttttaattttgctgaggctgttaacttctgcactgttgattggctgccattaggccgacagtgtgtggagcattatcgcttgcttcatcgatattgtgtgttttcccacgatgagatgatctgcaagatggcttccaaggctgatgtattagatgttgtagtggcttcaactgttcagaaagacatggccattatgattgaggatgagaaagctttaagagaaactgtccgtaaattgggagtgattgattcggaaagaatggattttgagctgttgccagatgatgaacgtcagtgtgtaaaatgcaaaactacatgcttcatgtctgccatctcctgttcttgtaaacctggccttcttgtttgcctgcatcatgtaaaagaattgtgttcctgtcctccttacaaatataaattgcggtataggtacacgctggatgatctctaccctatgatgaatgcattgaagcttcgagcagaatcttacaacgaatgggccttgaatgtgaatgaagctttggaggcaaagatcaacaagaagaaaagccttgtcagcttcaaggctttaattgaagaatctgaaatgaagaaattcccagacaatgatcttttgcgacaccttcgcctagtcacacaggatgcagagaagtgtgcctctgttgcgcagcagttgcttaatggcaaaaggcaaactagatatcgatctggtggagggaaatcccaaaatcagttgacagtgaatgagctccggcagtttgtaacacagctgtatgctcttccatgtgtcctcagtcagacaccattactaaaggatctcttgaatcgtgtagaagattttcaacagcatagtcagaaactactctctgaggaaacgcctagtgctgcggagctgcaggacttgctagatgtcagctttgaatttgatgttgaacttccacagcttgctgagatgcgtatccgtttggaacaagcccgttggctagaagaggtgcagcaagcttgcctagaccccagctcccttactttagatgatatgagacgtctcatagacctaggggtagggctggccccgtattcagcagtggagaaagctatggcccggctgcaggaactgctcacagtgtcagagcactgggacgacaaagccaagagtctcctcaaggccaggccacgacattcattgaatagccttgctacggcagtaaaggaaatcgaagagatccctgcatatctgcccaatggtgcggctctgaaagactcagtgcagagagccagagactggcttcaggatgtagagggcctgcaggctggaggacgtgtgccagtgttagacacactcatagaacttgttacacgaggccgatctatccccgtacatctgaattctttgccaagactggaaaccctagtagctgaggttcaggcttggaaagaatgtgctgttaatacattcttgactgagaattctccatattctctcttagaggtgctgtgtcctcgatgtgatattggccttttgggattgaaaaggaagcagagaaagttaaaggagcccttgccaaatggaaagaaaaaaagcaccaaattagagagtctgagtgacctggagagagctttaactgaaagcaaggagactgcttcagctatggcaactcttggggaagctcgcctaagggaaatggaagccttgcagtctctcagactcgccaatgaagggaaattgctgtcgcctctccaagatgtggatataaaaatctgcctatgtcagaaggccccagctgcccctatgattcaatgtgaactctgcagggatgctttccacaccagttgtgtggcggtacccagtatttcacagggcctgcgaatctggctttgtccccattgtcggaggtcagagaaacctccattagagaaaattctgcccctgctcgcctcccttcagcgtatccgagttcgccttcctgagggagatgcacttcgatatatgattgaaagaaccgtgaactggcagcacagagcccagcaactgctttcgtcagggaatcttaaatttgtgcaagatcgagtgggctcaggactgttatatagcagatggcaagcctcagcaggacaggtgtcagacacaaacaaggtatctcaacctcctggcacaacatcattttctttgcctgatgactgggacaacagaacctcatatttgcactcccccttctcaactggacgaagttgtatccccctccatggtgttagtccagaagtgaatgaactattgatggaagcccagctgctccaggtatcccttcctgaaattcaggaactttaccagactttacttgcaaagccaagccctgctcagcagactgaccgaagctcaccagtgagacccagcagtgagaagaatgactgttgccgagggaagcgagatggaattaacagtcttgagagaaaactgaagagacgcctggaaagagagggcctctccagtgagcggtgggaacgagttaagaaaatgcggacccccaaaaagaagaaaatcaaactgagccaccccaaggacatgaacaatttcaagttagagagagagcgtagctatgaattagttcgttctgctgaaactcattccctgccctcagacacatcctattccgaacaggaagactctgaggatgaagatgccatctgcccagctgtgagctgcctgcagccagaaggagatgaggtggactgggtccagtgtgatggcagctgcaatcagtggtttcatcaggtctgtgttggtgtctccccagagatggcagagaaagaagactacatctgtgtgcgctgtactgtgaaggacgcaccaagccgaaagtaaaaacacaaaaacagatacccccctacttaatgtaattcaggactccaaccaagaggatttcttcaaatctcagcaaagctacaggactggtactcaagccagcctgtaaacggtgctatttctattccttatgggatcatttttccaggactctttgaagaaaagaaaaaacaactaaaaaaatttttgacactttttgtattttttccttaagagctatttgtggttgttgaggtttgaaaagctgactgttttttttgcaggggttcccaccaatttggaaggcattgaagcttgcaccttttcatgtacagcattaaaattttacctctctctgggatttaccagcttaagagtccaactcacttccagtgcccaaaagggcacccaccagaaattccagtaaatcctcatttgaggaagctctcccttgtttactctgttaccacattggggaaatttttaagtttttcactttgggagtttttgtttgtttcttcttttcctttatccacttttcttcttcctggtagactaggtttatttatctgagcaataacttctatgttggtttcagtggctggaattaaaacaaaacaaaacaaacttccaaacagtgtgttggtgctttagcgattgattgatgtacagaacacaaatgtctagtttctagtgtcactgatgaactagtgatgtagaaaagagacttctctgtaagtaattgccacagctgtattttggctttctccctgtcccttcctttctcccctattttttggtagcttgtatcaaatgttacagtttatattgtggaataaatccttcgtcctaacataacactaaatgctgattatttagagccattagagcacagcttcttctgcccctctactgttgcacagccagaaggggctgcttgcttttgcctctgcccaaccaggttgcagtagcagtggatgttagcctgcaaacaacattaggggattcttcttttgtgtcctgctctgtttggtgggccatatgcctacagccctcactaacaaagaacccctctgtcttcaaggactagaacctatctttaaagccgtgctcttttaaaataagctttctcagaatgttggcaaatcacttcaatcctcaaatcagtcttccttgtggaatgctgcctttattatatttgaattgacaggggaacttggtttagggttaagacgttggaaggaaatctaaggaaaattaatcctcacagaggtccgggtttagtatatgtcttgagaggagacttgtgaattcccagactctgcctcctggtttcctttctcatttctttctaactgtagctatcattactggctgtggatagcccatagctattttccttgcttttcttttttaaaggggtctgttctgcagtggagaagacattctggtgaccagacttttgcttaccttttcctattctgttgccaatttttgttttccccacattctatagccataaacctgaagatgagtaaaactggtgggtctttaataaaacaacaacaaaaacagcagtttgtgatatagcagaggtttaaatgtaccctccccttttatgcacttcaaataattaaatctctttaagaatggaaaaaaaaaaaaaaaaaaaaaaaaaaaaaaaaaaaaa

SEQ ID NO: 324 - protein, 1544 aa, h. sapiens
MEAATTLHPGPRPALPLGGPGPLGEFLPPPECPVFEPSWEEFADPFAFIHKIRPIAEQTGICKVRPPPDWQPPFACDVDKLHFTPRIQRLNELEAQTRVKLNFLDQIAKYWELQGSTLKIPHVERKILDLFQLNKLVAEEGGFAVVCKDRKWTKIATKMGFAPGKAVGSHIRGHYERILNPYNLFLSGDSLRCLQKPNLTTDTKDKEYKPHDIPQRQSVQPSETCPPARRAKRMRAEAMNIKIEPEETTEARTHNLRRRMGCPTPKCENEKEMKSSIKQEPIERKDYIVENEKEKPKSRSKKATNAVDLYVCLLCGSGNDEDRLLLCDGCDDSYHTFCLIPPLHDVPKGDWRCPKCLAQECSKPQEAFGFEQAARDYTLRTFGEMADAFKSDYFNMPVHMVPTELVEKEFWRLVSTIEEDVTVEYGADIASKEFGSGFPVRDGKIKLSPEEEEYLDSGWNLNNMPVMEQSVLAHITADICGMKLPWLYVGMCFSSFCWHIEDHWSYSINYLHWGEPKTWYGVPGYAAEQLENVMKKLAPELFVSQPDLLHQLVTIMNPNTLMTHEVPVYRTNQCAGEFVITFPRAYHSGFNQGFNFAEAVNFCTVDWLPLGRQCVEHYRLLHRYCVFSHDEMICKMASKADVLDVVVASTVQKDMAIMIEDEKALRETVRKLGVIDSERMDFELLPDDERQCVKCKTTCFMSAISCSCKPGLLVCLHHVKELCSCPPYKYKLRYRYTLDDLYPMMNALKLRAESYNEWALNVNEALEAKINKKKSLVSFKALIEESEMKKFPDNDLLRHLRLVTQDAEKCASVAQQLLNGKRQTRYRSGGGKSQNQLTVNELRQFVTQLYALPCVLSQTPLLKDLLNRVEDFQQHSQKLLSEETPSAAELQDLLDVSFEFDVELPQLAEMRIRLEQARWLEEVQQACLDPSSLTLDDMRRLIDLGVGLAPYSAVEKAMARLQELLTVSEHWDDKAKSLLKARPRHSLNSLATAVKEIEEIPAYLPNGAALKDSVQRARDWLQDVEGLQAGGRVPVLDTLIELVTRGRSIPVHLNSLPRLETLVAEVQAWKECAVNTFLTENSPYSLLEVLCPRCDIGLLGLKRKQRKLKEPLPNGKKKSTKLESLSDLERALTESKETASAMATLGEARLREMEALQSLRLANEGKLLSPLQDVDIKICLCQKAPAAPMIQCELCRDAFHTSCVAVPSISQGLRIWLCPHCRRSEKPPLEKILPLLASLQRIRVRLPEGDALRYMIERTVNWQHRAQQLLSSGNLKFVQDRVGSGLLYSRWQASAGQVSDTNKVSQPPGTTSFSLPDDWDNRTSYLHSPFSTGRSCIPLHGVSPEVNELLMEAQLLQVSLPEIQELYQTLLAKPSPAQQTDRSSPVRPSSEKNDCCRGKRDGINSLERKLKRRLEREGLSSERWERVKKMRTPKKKKIKLSHPKDMNNFKLERERSYELVRSAETHSLPSDTSYSEQEDSEDEDAICPAVSCLQPEGDEVDWVQCDGSCNQWFHQVCVGVSPEMAEKEDYICVRCTVKDAPSRK

(19) United States
(12) Patent Application Publication
(10) Pub. No.: US 2003/0211521 A1
(43) Pub. Date: Nov. 13, 2003

(54) Breast cancer antigen

(75) Inventor: Joyce Taylor-Papadimitriou, London (GB)

Correspondence Address: NIXON & VANDERHYE, PC, 1100 N GLEBE ROAD, 8TH FLOOR, ARLINGTON, VA, 22201-4714, US

(73) Assignee: IMPERIAL CANCER RESEARCH TECHNOLOGY LIMITED

(21) Appl. No.: 10/334,726

(22) Filed: Jan. 2, 2003

Related U.S. Application Data
(63) Continuation of application No. 09/645,446, filed on Aug. 25, 2000, now abandoned.
Application No. 09/645,446 is a continuation of application No. PCT/GB99/00866, filed on Mar. 19, 1999.

(30) Foreign Application Priority Data
Mar. 20, 1998 (GB) 9805877.9

(51) Int. Cl. C12Q 1/68; G01N 33/574; C07H 21/04; C12P 21/02; C12N 5/06; C07K 14/47; C07K 16/30

(52) U.S. Cl. 435/6; 435/7.23; 435/69.1; 435/320.1; 435/325; 530/350; 530/388.8; 536/23.5

(57) ABSTRACT

A gene encoding a polypeptide, related in sequence to retinoblastoma binding proteins 1 and 2, which is expressed in breast cancer, therapeutic and diagnostic methods relating to this gene and polypeptide.

The present invention relates to cancer and in particular to breast cancer.

Cancer is a serious disease and a major killer. Although there have been advances in the diagnosis and treatment of certain cancers in recent years, there is still a need for improvements in diagnosis and treatment.

Cancer is a genetic disease and in most cases involves mutations in one or more genes. There are believed to be around 200,000 genes in the human genome but only a handful of these genes have been shown to be involved in cancer. Although it is surmised that many more genes than have been presently identified will be found to be involved in cancer, progress in this area has remained slow despite the availability of molecular analytical techniques. This may be due to the varied structure and function of genes which have been identified to date which suggests that cancer genes can take many forms and have many different functions.

Breast cancer is one of the most significant diseases that affects women. At the current rate, American women have a 1 in 8 risk of developing cancer by the age of 95 (American Cancer Society, Cancer Facts and Figures, 1992, American Cancer Society, Atlanta, Ga., USA). Genetic factors contribute to an ill-defined proportion of breast cancer cases, estimated to be about 5% of all cases but approximately 25% of cases diagnosed before the age of 40 (Claus et al (1991) Am J. Hum. Genet. 48, 232-242). Breast cancer has been divided into two types, early-age onset and late stage onset, based on an inflection in the age-specific incidence curve at around the age of 50. Mutation of one gene, BRCA1, is thought to account for approximately 45% of familial breast cancer, but at least 80% of families with both breast and ovarian cancer (Easton et al (1993) Am. J. Hum. Genet. 52, 678-701).

Ovarian cancer is the most frequent cause of death from gynaecological malignancies in the Western World, with an incidence of 5,000 new cases every year in England and Wales. It is the fourth most common cause of cancer mortality in American women. The majority of patients with epithelial ovarian cancer present at an advanced stage of the disease. Consequently, the 5 year survival rate is only 30% after adequate surgery and chemotherapy despite the introduction of new drugs such as platinum and taxol (Advanced Ovarian Cancer Trialists Group (1991) BMJ 303, 884-893; Ozols (1995) Semin Oncol. 22, 61-66). However, patients who have stage I disease (confined to the ovaries) do better with the 5 year survival rate being 70%. It is therefore desirable to have techniques to detect the cancer before metastasis to have a significant impact on survival.

Epithelial ovarian cancer constitutes 70-80% of ovarian cancer and encompasses a broad spectrum of lesions, ranging from localized benign tumours and neoplasms of borderline malignant potential to invasive adenocarcinomas. Histologically, the common epithelial ovarian cancers, are classified into several types, that is, serous, mucinous, endometrioid, clear cell, Brenner, mixed epithelial, and undifferentiated tumours. The heterogeneity of histological subtypes reflects the metaplastic potential of the ovarian surface Mullerian epithelium which shares a common embryological origin with the peritoneum and the rest of the uro-genital system. Germ cell, sex cord/stromal tumours and sarcomas represent the remainder of ovarian cancers. The histogenesis and biological characteristics of epithelial ovarian cancer are poorly understood as are the molecular genetic alterations that may contribute to the development of such tumours or their progression. Epidemiological factors related to ovulation seem to be important, whereby ovarian epithelial cells undergo several rounds of division and proliferative growth to heal the wound in the epithelial surface. These lead to the development of epithelial inclusion cysts and frank malignant tumours may arise from them (Fathalla (1971) Lancet 2, 163).

Despite the recent interest in the breast cancer predisposing genes, BRCA1 and BRCA2, there remains the need for further information on breast cancer, and the need for further diagnostic markers and targets for therapeutic intervention. Recently, the role of tumour-associated antigens in the biology of cancer has begun to be investigated. Probably the best studied example of tumour-associated antigens are the MAGE antigens which are involved in melanoma and certain other cancers, such as breast cancer. Therapeutic and diagnostic approaches making use of the MAGE antigens are described in Gattoni-Celli & Cole (1996) Seminars in Oncology 23, 754-758, Itoh et al (1996) J. Biochem. 119, 385-390, WO 92/20356, WO 94/23031, WO 94/05304, WO 95/20974 and WO 95/23874. However, other tumour-associated antigens have also been implicated in breast cancer. For example, studies concerning the antigens expressed by breast cancer cells, and in particular how these relate to the antigenic profile of the normal mammary epithelial cell, have been and continue to be a major activity in breast cancer research. The role of certain antigens in breast cancer, especially the role of polymorphic epithelial mucin (PEM; the product of the MUC 1 gene) and the c-erbB2 protooncogene, are reviewed in Taylor-Papadimitriou et al (1993) Annals NY Acad. Sci. 698, 31-47. Other breast cancer associated antigens include MAGE-1 and CEA.

Immunotherapeutic strategies and vaccines involving the MUC1 gene or PEM are described in Burchell et al (1996), pp 309-313, In Breast Cancer, Advances in Biology and Therapeutics, Calvo et al (eds), John Libbey Eurotext; Graham et al (1996) Int. J. Cancer 65, 664-670; Graham et al (1995) Tumor Targeting 1, 211-221; Finn et al (1995) Immunol. Rev. 145, 61-89; Burchell et al (1993) Cancer Surveys 18, 135-148; Scholl & Pouillart (1997) Bull. Cancer 84, 61-64; and Zhang et al (1996) Cancer Res. 56, 3315-3319.

Defeo-Jones et al (1991) Nature 352, 251-254 describes the cloning of cDNAs for cellular proteins that bind to the retinoblastoma gene product (RB); Fattaey et al (1993) Oncogene 8, 3149-3156 describes the characterisation of the retinoblastoma binding proteins RBP1 and RBP2; Wu et al (1994) Hum. Mol. Genet. 3, 153-160 describes the isolation and characterization of XE 169, a human gene that escapes X inactivation; Agulnik et al (1994) Hum. Mol. Genet. 3, 879-884 describes an X chromosome gene, with a widely transcribed Y-linked homologue, which escapes X-inactivation in mouse and human; and various expressed sequence tags (ESTs) which have been designated as being derived from a gene called RBP3 have been described in the GenBank database.

None of these genes have been shown to be associated with cancer. There remains a need for the identification of further tumour-associated antigens, especially breast cancer-associated antigens since immunotherapeutic treatments may be HLA-type specific and a single tumour antigen may not be useful in all cases.

I have now, surprisingly, found that a gene encoding a polypeptide which has similarity to the retinoblastoma binding proteins (RBPs), and also has similarity to the polypeptides encoded by the genes described in Wu et al supra and Agulnik et al supra, is associated with breast cancer and probably also with ovarian cancer. In particular, the mRNA and polypeptide encoded by the gene, which I have called plu-1, is present in breast cancer cells. The plu-1 antigen appears to be more ubiquitously expressed in breast tumours than some existing tumour antigens.

I have isolated the full length plu-I cDNA. Partial length and incomplete cDNAs which seem to be derived from the same gene appear to be known as expressed sequence tags (ESTs) as is described in more detail below, but the present patent application is, as far as I am aware, the first disclosure of the full length coding sequence. The plu-1 polypeptide has not, as far as I am aware, been described previously.

As is discussed more fully below, the plu-1 cDNA and polypeptide share some similarity to RBP-1 and RBP-2. In addition, portions of the plu-1 cDNA share substantially complete identity with various ESTs and other sequences in the database. One particular sequence (HSU50848) has been labelled “RBP-3” in the GenBank database on the basis of its similarity to RBP-1 and RBP-2; however, I have found from the complete plu-1 cDNA sequence and encoded polypeptide that the RB-binding motify (LXCXE) is absent from plu-1 and so it seems unlikely that the plu-1 polypeptide binds RB.

An object of the invention is to provide a full length cDNA for plu-1 and thereby provide a polypeptide encoded by the plu-1 cDNA and gene.

Further objects of the invention include the provision of peptide fragments of the plu-1 polypeptide and plu-1 polynucleotides which are useful for raising an immune response.

Still further objects of the invention include the provision of antibodies which are selective for the plu-1 polypeptide; and uses of such antibodies for diagnostic and other methods; the provision of diagnostic and therapeutic methods which involve the plu-1 gene, cDNA or polypeptide or portions thereof; and cancer vaccines which make use of the plu-gene, cDNA or polypeptide or portions thereof.

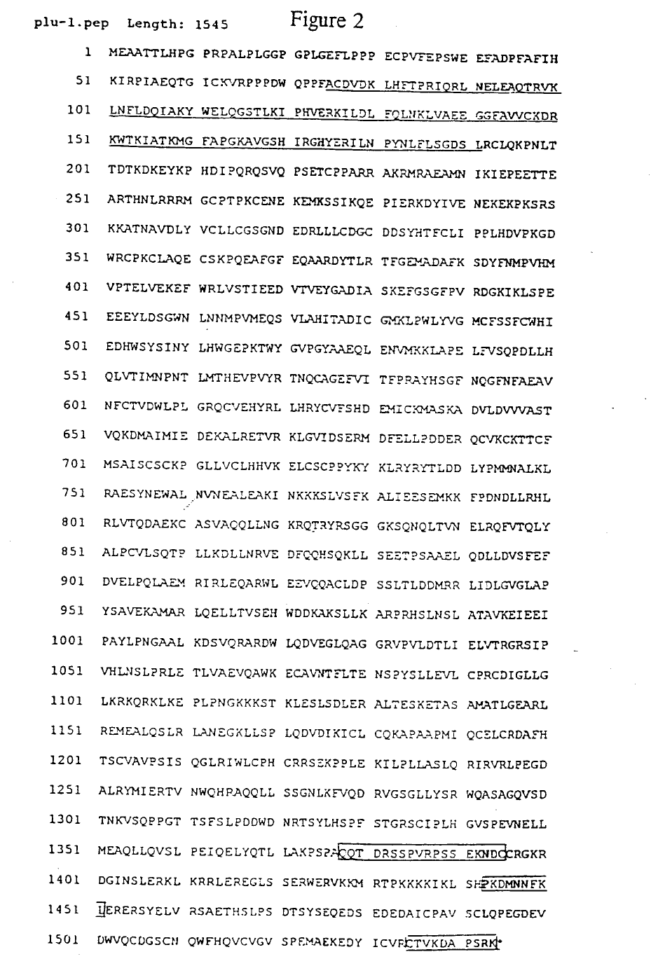

A first aspect of the invention provides a recombinant polynucleotide encoding a polypeptide comprising the amino acid sequence shown in FIG. 2 or variants or fragments or fusions or derivatives thereof, or fusions of said variants or fragments or derivatives. The amino acid sequence shown in FIG. 2 is that of the plu-1 polypeptide.

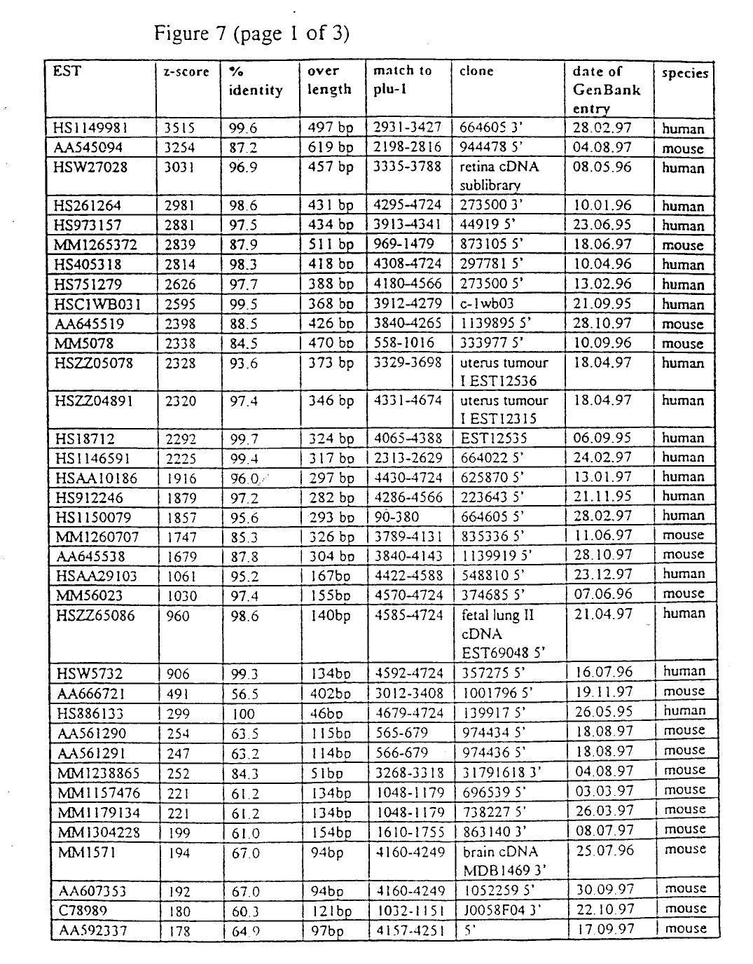

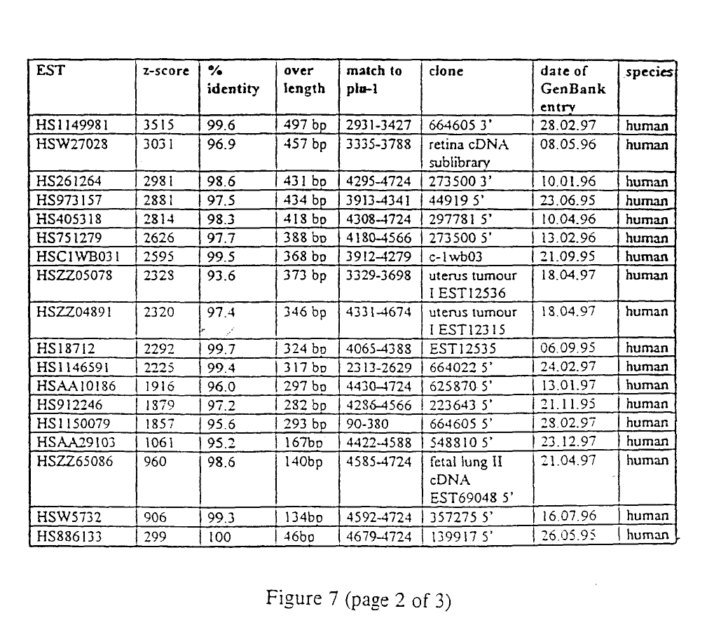

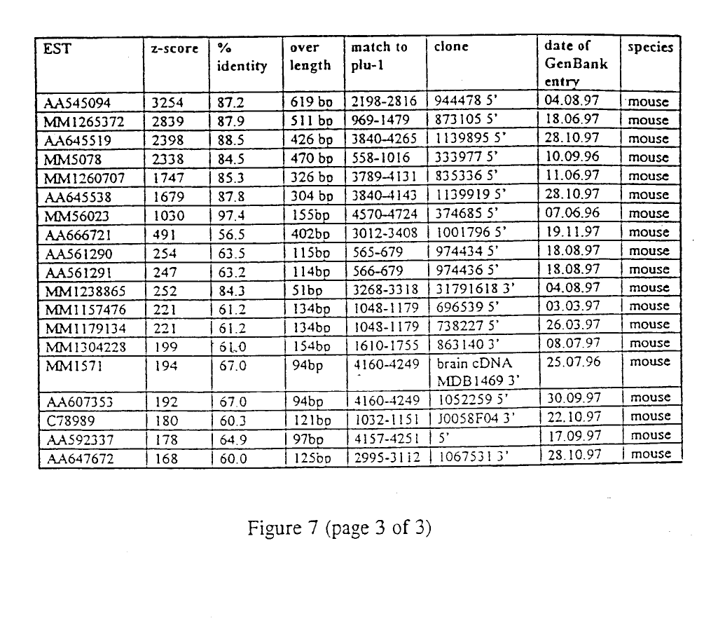

The invention does not include the recombinant polynucleotides per se which are disclosed in GenBank and which are related to the plu-1 cDNA. These include polynucleotides disclosed by reference to the GenBank accession details shown in FIG. 7 and the details of the clone described under GenBank accession no HSU50848 (called “RBP-3”) and a clone related to plu-1 described in GenBank accession no KIAA0234.

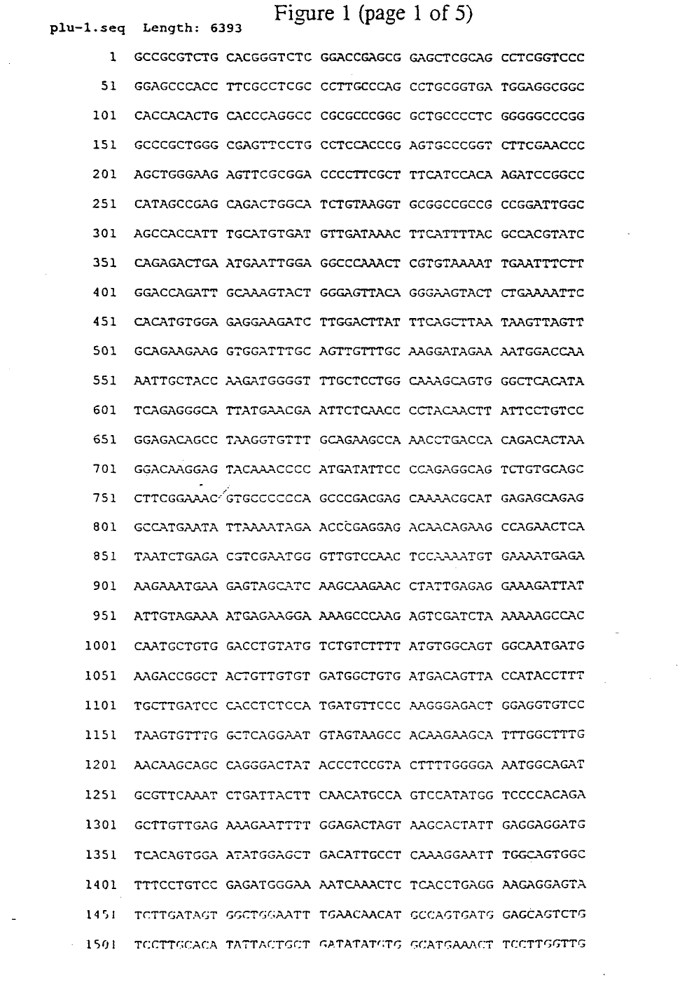

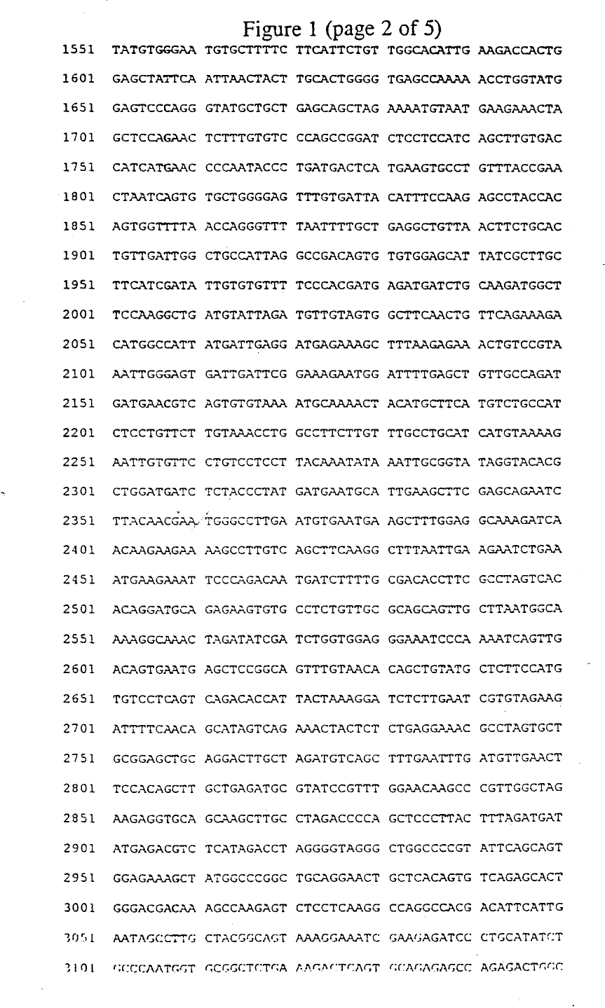

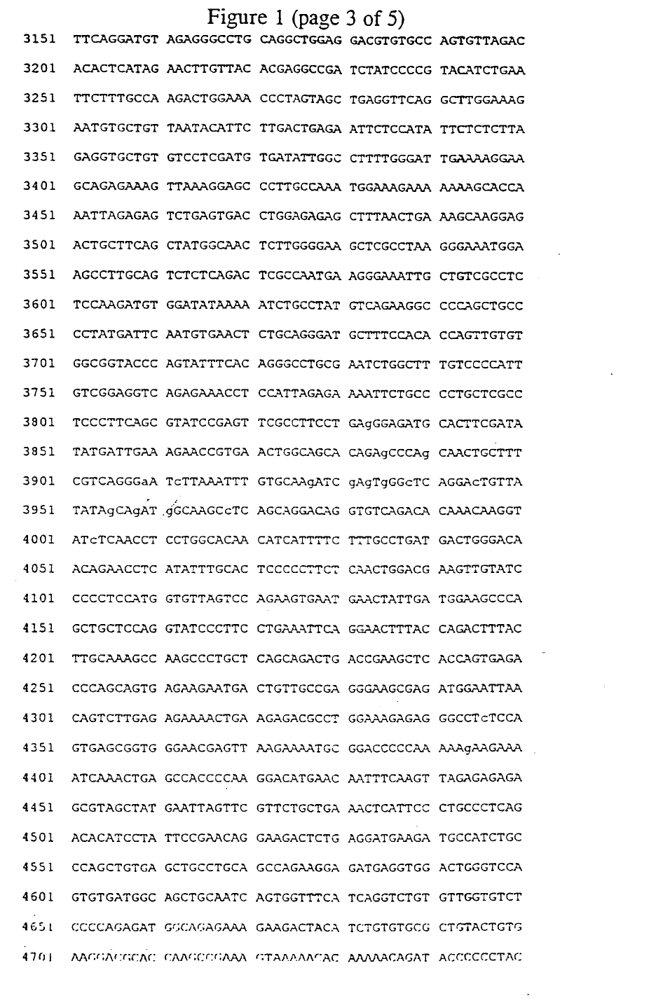

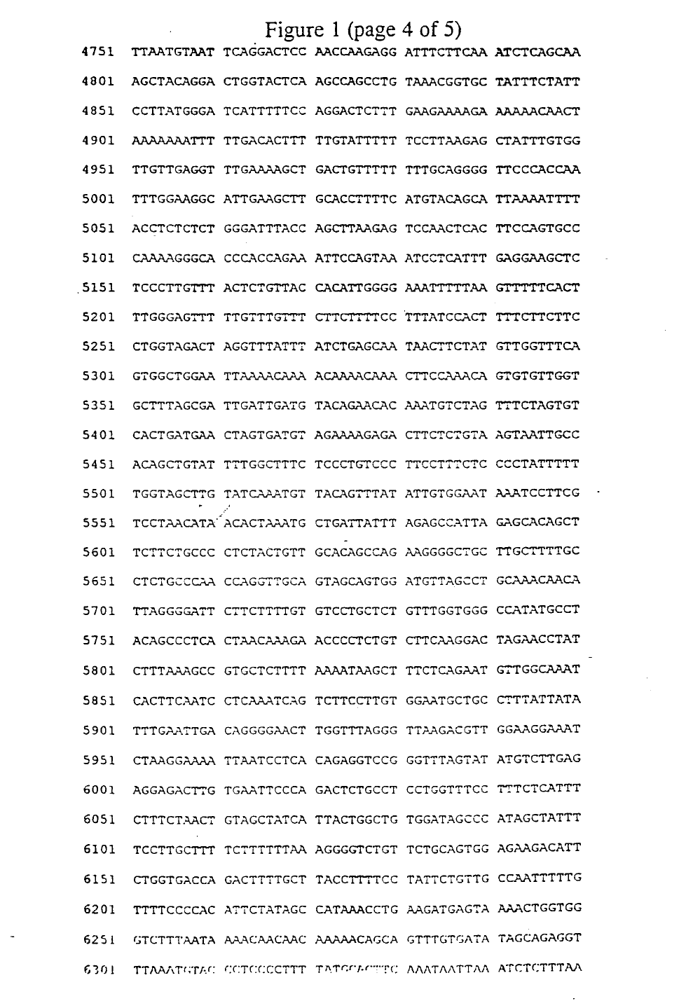

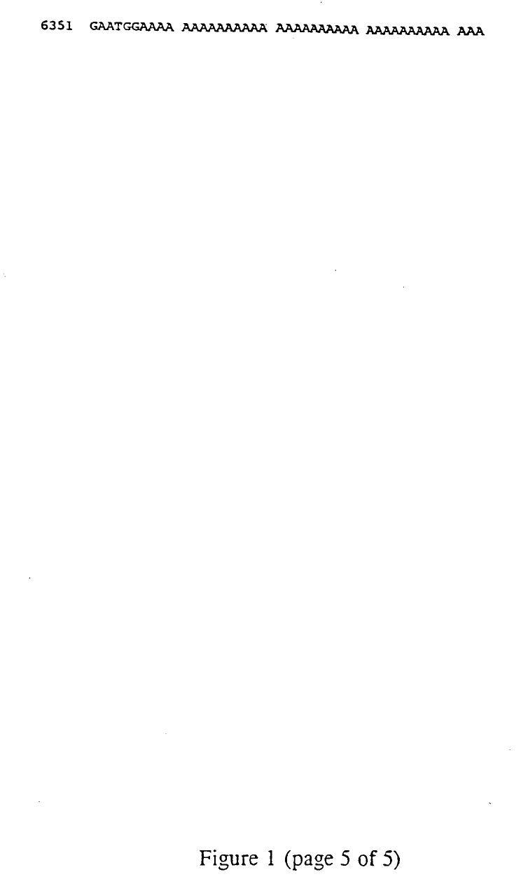

FIG. 2 shows the amino acid sequence encoded by the cDNA insert shown in FIG. 1.

Throughout the specification where the term plu-1 is used, and the context does not indicate otherwise, it includes as appropriate the polypeptide which has the amino acid sequence given in FIG. 2 or the cDNA whose sequence is given in FIG. 1 (more particularly the coding sequence thereof which is found from positions 90 to 4724) or the gene which encodes the plu-1 polypeptide.

It will be appreciated that a plu-1-encoding cDNA may be readily obtained using the methods described in the Examples or by using a suitable probe derived from the FIG. 1 nucleotide sequence to screen a human cDNA library at high stringency. The plu-1 amino acid sequence may readily be deduced from the full length cDNA sequence.

Amino acid residues are given in standard single letter code or standard three letter code throughout the specification.

It will be appreciated that the recombinant polynucleotides per se of the invention do not include polynucleotides which encode retinoblastoma binding protein-1 (RBP-1) or retinoblastoma binding protein-2 (RBP-2) or the polynucleotides associated with the GenBank accession no HSU50848 designated “RBP-3” or the other polynucleotides identified above.

Preferably, the fragments and variants and derivatives are those that include a polynucleotide which encodes a portion or portions of plu-1 which are portions that distinguish plu-1 from RBP-1, RBP-2, the portions of “RBP-3” (as designated) which are described by reference to FIG. 7 and other polypeptides encoded by the polynucleotides identified by reference to FIG. 7 and which are described in more detail below and by reference to FIG. 2.

The polynucleotide may be DNA or RNA but it is preferred if it is DNA. The polynucleotide may or may not contain introns. It is preferred that it does not contain introns and it is particularly preferred if the polynucleotide is a cDNA. A polynucleotide of the invention includes the plu-1 gene which may be obtained using a suitable gene library (such as a human YAC or PAC or cosmid library, particularly one which includes DNA from human chromosome 1) and a probe derived from the plu-1 cDNA. By “plu-1 gene” we include elements associated with the plu-1 coding region which are involved in control of plu-1 expression, such as regions which are susceptible to methylation (eg CpG islands).

A polynucleotide of the invention is one which comprises the polynucleotide whose sequence is given in FIG. 1. Thus, a polynucleotide of the invention includes the one with the sequence shown in FIG. 1.

It is particularly preferred if the polynucleotide of the invention is one which comprises the polynucleotide whose sequence is given between positions 90 and 4724 in FIG. 1 since this is believed to be the coding sequence for the plu-1 polypeptide.

The invention includes a polynucleotide comprising a fragment of the recombinant polynucleotide of the first aspect of the invention. Preferably, the polynucleotide comprises a fragment which is at least 10 nucleotides in length, more preferably at least 14 nucleotides in length and still more preferably at least 18 nucleotides in length. Such polynucleotides are useful as PCR primers.

A “variation” of the polynucleotide includes one which is (i) usable to produce a protein or a fragment thereof which is in turn usable to prepare antibodies which specifically bind to the protein encoded by the said polynucleotide or (ii) an antisense sequence corresponding to the gene or to a variation of type (i) as just defined. For example, different codons can be substituted which code for the same amino acid(s) as the original codons. Alternatively, the substitute codons may code for a different amino acid that will not affect the activity or immunogenicity of the protein or which may improve or otherwise modulate its activity or immunogenicity. For example, site-directed mutagenesis or other techniques can be employed to create single or multiple mutations, such as replacements, insertions, deletions, and transpositions, as described in Botstein and Shortle, “Strategies and Applications of In Vitro Mutagenesis,” Science, 229: 193-210 (1985), which is incorporated herein by reference. Since such modified polynucleotides can be obtained by the application of known techniques to the teachings contained herein, such modified polynucleotides are within the scope of the claimed invention.

Moreover, it will be recognised by those skilled in the art that the polynucleotide sequence (or fragments thereof) of the invention can be used to obtain other polynucleotide sequences that hybridise with it under conditions of high stringency. Such polynucleotides include any genomic DNA. Accordingly, the polynucleotide of the invention includes polynucleotides that shows at least 90 percent, preferably 95 percent, and more preferably at least 99 percent and most preferably at least 99.9 percent homology with the plu-1 polynucleotide shown in FIG. 1, provided that such homologous polynucleotide encodes a polypeptide which is usable in at least some of the methods described below or is otherwise useful. It is particularly preferred that in this embodiment, the polynucleotide is one which encodes a polypeptide containing a portion or portions that distinguish plu-1 from any of RBP-1, RBP-2, and the other polypeptides encoded by the polynucleotides identified by reference to FIG. 7.

It is believed that plu-1 is found in mammals other than human. The present invention therefore includes polynucleotides which encode plu-1 from other mammalian species including rat, mouse, cow, pig, sheep, rabbit and so on.

Percent homology can be determined by, for example, the GAP program of the University of Wisconsin Genetic Computer Group.

DNA-DNA, DNA-RNA and RNA-RNA hybridisation may be performed in aqueous solution containing between 0.1×SSC and 6×SSC and at temperatures of between 55° C. and 70° C. It is well known in the art that the higher the temperature or the lower the SSC concentration the more stringent the hybridisation conditions. By “high stringency” we mean 2×SSC and 65° C. 1×SSC is 0.15M NaCl/0.015M sodium citrate. Polynucleotides which hybridise at high stringency are included within the scope of the claimed invention.

“Variations” of the polynucleotides also include polynucleotide in which relatively short stretches (for example 20 to 50 nucleotides) have a high degree of homology (at least 90% and preferably at least 99 or 99.9%) with equivalent stretches of the polynucleotide of the invention even though the overall homology between the two polynucleotides may be much less. This is because important active or binding sites may be shared even when the general architecture of the protein is different.

By “variants” of the polypeptide we include insertions, deletions and substitutions, either conservative or non-conservative, where such changes do not substantially alter the activity of the said plu-1.

Variants and variations of the polynucleotide and polypeptide include natural variants, including allelic variants and naturally-occurring mutant forms.

By “conservative substitutions” is intended combinations such as Gly, Ala; Val, Ile, Leu; Asp; Glu; Asn, Gln; Ser, Thr; Lys, Arg; and Phe, Tyr.

Such variants may be made using the methods of protein engineering and site-directed mutagenesis well known in the art.

Preferably, the variant or variation of the polynucleotide encodes a plu-1 that has at least 30%, preferably at least 50% and more preferably at least 70% of the activity of a natural plu-1, under the same assay conditions.

Analysis of the plu-1 polypeptide suggests that it may be involved in binding DNA and in modulating transcription. The polypeptide contains three PHD finger motifs (positions 309-359, 1176-1224 and 1484-1538) suggesting that it may bind to chromatin and change its structure, thereby modulating transcriptional activity. The plu-1 polypeptide may be involved in regulating the transcription of a number of genes and it may have a nuclear localization. Bipartite nuclear localisation signals are found at positions 1102-1119 and 1399-1416. A further proposed DNA binding motif, the dead ringer domain, stretches from amino acids 75-191 and is underlined in FIG. 2.

A review of PHD fingers is given in Aasland et al (1995) Trends Biochem. Sci. 20, 56-59.

By “fragment of plu-1” we include any fragment which retains activity or which is useful in some other way, for example, for use in raising antibodies or in a binding assay. Preferably, the fragment of plu-1 is not a fragment of plu-1 which could also be a fragment of RBP-1 or RPB-2 or any other polypeptides encoded by the polynucleotides identified by reference to FIG. 7.

By “fusion of plu-1” we include said plu-1 fused to any other polypeptide. For example, the said plu-1 may be fused to a polypeptide such as glutathione-S-transferase (GST) or protein A in order to facilitate purification of plu-1, or it may be fused to some other polypeptide which imparts some desirable characteristics on the plu-1 fusion. Fusions to any variant, fragment or derivative of plu-1 are also included in the scope of the invention.

For the avoidance of doubt, I believe that a clone containing a full-length coding region for plu-i has not been disclosed previously and that there has been no suggestion that plu-1 may be a tumour-associated antigen. Thus, in relation to all of the methods using plu-i cDNAs or genes or polypeptides or variants or fragments or derivatives or fusions thereof, or fusions of said variants, fragments or derivatives, known materials may be used in these methods as well as the new materials disclosed herein.

A further aspect of the invention provides a replicable vector comprising a recombinant polynucleotide encoding plu-1, or a variant, fragment, derivative or fusion of plu-1 or a fusion of said variant, fragment or derivative.

A variety of methods have been developed to operably link polynucleotides, especially DNA, to vectors for example via complementary cohesive termini. For instance, complementary homopolymer tracts can be added to the DNA segment to be inserted to the vector DNA. The vector and DNA segment are then joined by hydrogen bonding between the complementary homopolymeric tails to form recombinant DNA molecules.

Synthetic linkers containing one or more restriction sites provide an alternative method of joining the DNA segment to vectors. The DNA segment, generated by endonuclease restriction digestion as described earlier, is treated with bacteriophage T4 DNA polymerase or E. coli DNA polymerase I, enzymes that remove protruding, 3′-single-stranded termini with their 3′-5′-exonucleolytic activities, and fill in recessed 3′-ends with their polymerizing activities.

The combination of these activities therefore generates blunt-ended DNA segments. The blunt-ended segments are then incubated with a large molar excess of linker molecules in the presence of an enzyme that is able to catalyze the ligation of blunt-ended DNA molecules, such as bacteriophage T4 DNA ligase. Thus, the products of the reaction are DNA segments carrying polymeric linker sequences at their ends. These DNA segments are then cleaved with the appropriate restriction enzyme and ligated to an expression vector that has been cleaved with an enzyme IS that produces termini compatible with those of the DNA segment.

Synthetic linkers containing a variety of restriction endonuclease sites are commercially available from a number of sources including International Biotechnologies Inc, New Haven, CN, USA.

A desirable way to modify the DNA encoding the polypeptide of the invention is to use the polymerase chain reaction as disclosed by Saiki et al (1988) Science 239, 487491. This method may be used for introducing the DNA into a suitable vector, for example by engineering in suitable restriction sites, or it may be used to modify the DNA in other useful ways as is known in the art.

In this method the DNA to be enzymatically amplified is flanked by two specific primers which themselves become incorporated into the amplified DNA. The said specific primers may contain restriction endonuclease recognition sites which can be used for cloning into expression vectors using methods known in the art.

The DNA (or in the case of retroviral vectors, RNA) is then expressed in a suitable host to produce a polypeptide comprising the compound of the invention. Thus, the DNA encoding the polypeptide constituting the compound of the invention may be used in accordance with known techniques, appropriately modified in view of the teachings contained herein, to construct an expression vector, which is then used to transform an appropriate host cell for the expression and production of the polypeptide of the invention. Such techniques include those disclosed in U.S. Pat. No. 4,440,859 issued Apr. 3, 1984 to Rutter et al, U.S. Pat. No. 4,530,901 issued Jul. 23, 1985 to Weissman, U.S. Pat. No. 4,582,800 issued Apr. 15, 1986 to Crowl, U.S. Pat. No. 4,677,063 issued Jun. 30, 1987 to Mark et al, U.S. Pat. No. 4,678,751 issued Jul. 7, 1987 to Goeddel, U.S. Pat. No. 4,704,362 issued Nov. 3, 1987 to Itakura et al, U.S. Pat. No. 4,710,463 issued Dec. 1, 1987 to Murray, U.S. Pat. No. 4,757,006 issued Jul. 12, 1988 to Toole, Jr. et al, U.S. Pat. No. 4,766,075 issued Aug. 23, 1988 to Goeddel et al and U.S. Pat. No. 4,810,648 issued Mar. 7, 1989 to Stalker, all of which are incorporated herein by reference.

The DNA (or in the case of retroviral vectors, RNA) encoding the polypeptide constituting the compound of the invention may be joined to a wide variety of other DNA sequences for introduction into an appropriate host. The companion DNA will depend upon the nature of the host, the manner of the introduction of the DNA into the host, and whether episomal maintenance or integration is desired.

Generally, the DNA is inserted into an expression vector, such as a plasmid, in proper orientation and correct reading frame for expression. If necessary, the DNA may be linked to the appropriate transcriptional and translational regulatory control nucleotide sequences recognised by the desired host, although such controls are generally available in the expression vector. The vector is then introduced into the host through standard techniques. Generally, not all of the hosts will be transformed by the vector. Therefore, it will be necessary to select for transformed host cells. One selection technique involves incorporating into the expression vector a DNA sequence, with any necessary control elements, that codes for a selectable trait in the transformed cell, such as antibiotic resistance. Alternatively, the gene for such selectable trait can be on another vector, which is used to co-transform the desired host cell.

Host cells that have been transformed by the recombinant DNA of the invention are then cultured for a sufficient time and under appropriate conditions known to those skilled in the art in view of the teachings disclosed herein to permit the expression of the polypeptide, which can then be recovered.

Many expression systems are known, including bacteria (for example E. coli and Bacillus subtilis), yeasts (for example Saccharomyces cerevisiae), filamentous fungi (for example Aspergillus), plant cells, animal cells and insect cells.

The vectors typically include a prokaryotic replicon, such as the ColE1 ori, for propagation in a prokaryote, even if the vector is to be used for expression in other, non-prokaryotic, cell types. The vectors can also include an appropriate promoter such as a prokaryotic promoter capable of directing the expression (transcription and translation) of the genes in a bacterial host cell, such as E. coli, transformed therewith.

A promoter is an expression control element formed by a DNA sequence that permits binding of RNA polymerase and transcription to occur. Promoter sequences compatible with exemplary bacterial hosts are typically provided in plasmid vectors containing convenient restriction sites for insertion of a DNA segment of the present invention.

Typical prokaryotic vector plasmids are pUC18, pUC19, pBR322 and pBR329 available from Biorad Laboratories, (Richmond, Calif., USA) and pTrc99A and pKK223-3 available from Pharmacia, Piscataway, N.J., USA.

A typical mammalian cell vector plasmid is pSVL available from Pharmacia, Piscataway, N.J., USA. This vector uses the SV40 late promoter to drive expression of cloned genes, the highest level of expression being found in T antigen-producing cells, such as COS-1 cells.

An example of an inducible mammalian expression vector is pMSG, also available from Pharmacia. This vector uses the glucocorticoid-inducible promoter of the mouse mammary tumour virus long terminal repeat to drive expression of the cloned gene.

Useful yeast plasmid vectors are pRS403406 and pRS413416 and are generally available from Stratagene Cloning Systems, La Jolla, Calif. 92037, USA. Plasmids pRS403, pRS404, pRS405 and pRS406 are Yeast Integrating plasmids (YIps) and incorporate the yeast selectable markers HIS3, TRP1, LEU2 and URA3. Plasmids pRS413416 are Yeast Centromere plasmids (Ycps).

Other vectors and expression systems are well known in the art for use with a variety of host cells.

From the foregoing it will be appreciated that a particularly preferred embodiment of the invention is an expression vector which is capable of expressing in a mammalian, preferably human, cell a polypeptide having the amino acid sequence shown in FIG. 2 or variants or fragments or derivatives thereof, or fusions of said variants or fragments or derivatives.

The present invention also relates to a host cell transformed with a polynucleotide vector construct of the present invention. The host cell can be either prokaryotic or eukaryotic. Bacterial cells may be preferred prokaryotic host cells in some circumstances and typically are a strain of E. coli such as, for example, the E. coli strains DH5 available from Bethesda Research Laboratories Inc., Bethesda, Md., USA, and RR1 available from the American Type Culture Collection (ATCC) of Rockville, Md., USA (No ATCC 31343). Preferred eukaryotic host cells include yeast, insect and mammalian cells, preferably vertebrate cells such as those from a mouse, rat, monkey or human fibroblastic and kidney cell lines. Yeast host cells include YPH499, YPH500 and YPH501 which are generally available from Stratagene Cloning Systems, La Jolla, Calif. 92037, USA Preferred mammalian host cells include Chinese hamster ovary (CHO) cells available from the ATCC as CCL61, NIH Swiss mouse embryo cells NIH/3T3 available from the ATCC as CRL 1658, monkey kidney-derived COS-1 cells available from the ATCC as CRL 1650 and 293 cells which are human embryonic kidney cells. Preferred insect cells are Sf9 cells which can be transfected with baculovirus expression vectors.

Transformation of appropriate cell hosts with a DNA construct of the present invention is accomplished by well known methods that typically depend on the type of vector used. With regard to transformation of prokaryotic host cells, see, for example, Cohen et al (1972) Proc. Natl. Acad. Sci. USA 69, 2110 and Sambrook et al (1989) Molecular Cloning, A Laboratory Manual, Cold Spring Harbor Laboratory, Cold Spring Harbor, N.Y. Transformation of yeast cells is described in Sherman et al (1986) Methods In Yeast Genetics, A Laboratory Manual, Cold Spring Harbor, N.Y. The method of Beggs (1978) Nature 275, 104-109 is also useful. With regard to vertebrate cells, reagents useful in transfecting such cells, for example calcium phosphate and DEAE-dextran or liposome formulations, are available from Stratagene Cloning Systems, or Life Technologies Inc., Gaithersburg, Md. 20877, USA.

Electroporation is also useful for transforming and/or transfecting cells and is well known in the art for transforming yeast cell, bacterial cells, insect cells and vertebrate cells.

For example, many bacterial species may be transformed by the methods described in Luchansky et al (1988) Mol. Microbiol. 2, 637-646 incorporated herein by reference. The greatest number of transformants is consistently recovered following electroporation of the DNA-ell mixture suspended in 2.5×PEB using 6250V per cm at 25 μFD.

Methods for transformation of yeast by electroporation are disclosed in Becker & Guarente (1990) Methods Ezymol. 194, 182.

Successfully transformed cells, ie cells that contain a DNA construct of the present invention, can be identified by well known techniques. For example, cells resulting from the introduction of an expression construct of the present invention can be grown to produce the polypeptide of the invention. Cells can be harvested and lysed and their DNA content examined for the presence of the DNA using a method such as that described by Southern (1975) J. Mol. Biol. 98, 503 or Berent et al (1985) Biotech. 3, 208. Alternatively, the presence of the protein in the supernatant can be detected using antibodies as described below.

In addition to directly assaying for the presence of recombinant DNA, successful transformation can be confirmed by well known immunological methods when the recombinant DNA is capable of directing the expression of the protein. For example, cells successfully transformed with an expression vector produce proteins displaying appropriate antigenicity. Samples of cells suspected of being transformed are harvested and assayed for the protein using suitable antibodies. The cells which are transformed are preferably mammary epithelial cells.

Thus, in addition to the transformed host cells themselves, the present invention also contemplates a culture of those cells, preferably a monoclonal (clonally homogeneous) culture, or a culture derived from a monoclonal culture, in a nutrient medium.

Particularly when the plu-1 nucleic acid is a fragment of the sequence shown in FIG. 1, it is preferred if the host cell is not a bacterial cell.

It is particularly preferred if the host cell is an animal cell, more preferably a mammalian cell.

It is particularly preferred if the plu-1 polynucleotide is prepared into a pharmaceutical composition and is sterile and pyrogen-free.

A further aspect of the invention provides a method of making plu-1 or a variant, derivative, fragment or fusion thereof or a fusion of a variant, fragment or derivative, the method comprising culturing a host cell comprising a recombinant polynucleotide or a replicable vector which encodes said plu-1 or variant or fragment or derivative or fusion, and isolating said plu-1 or a variant, derivative, fragment or fusion thereof of a fusion or a variant, fragment or derivative from said host cell.

Methods of cultivating host cells and isolating recombinant proteins are well known in the art. It will be appreciated that, depending on the host cell, the plu-1 produced may differ from that which can be isolated from nature. For example, certain host cells, such as yeast or bacterial cells, either do not have, or have different, post-translational modification systems which may result in the production of forms of plu-1 which may be post-translationally modified in a different way to plu-1 isolated from nature.

It is preferred that recombinant plu-1 is produced in a eukaryotic system, such as an insect cell.

A further aspect of the invention provides plu-1 or a variant, fragment, derivative or fusion thereof or a fusion of a variant, fragment or derivative obtainable by the methods herein disclosed.

A further aspect of the invention provides a polypeptide comprising the amino acid sequence shown in FIG. 2 or variants or fragments or fusions or derivatives thereof or fusions of said variants or fragments or derivatives.

Thus, a polypeptide of the invention includes the polypeptide whose amino acid sequence is shown in FIG. 2.

It will be appreciated that the polypeptides of the invention do not include RBP-1 or RBP-2. Preferably, the fragments and variants and derivatives are those that include a portion or portions of plu-I which are portions that distinguish plu-1 from RBP-1, RBP-2 or polypeptides encoded by polynucleotides identified by reference to FIG. 7 and which are described in more detail below and by reference to FIG. 2.

A further aspect of the invention provides antibodies which are selective for plu-1 (and do not cross react with, for example, RBP-1, RBP-2).

By “selective” we include antibodies which bind at least 10-fold more strongly to one polypeptide than to the other (ie plu-1 vs RBP-1 or RBP-2); preferably at least 50-fold more strongly and more preferably at least 100-fold more strongly.

Such antibodies may be made by methods well known in the art using the information concerning the differences in amino acid sequence between plu-1 and RBP-1, RBP-2 and other polypeptides encoded by polynucleotides identified by reference to FIG. 7 disclosed herein. In particular, the antibodies may be polyclonal or monoclonal.

Suitable monoclonal antibodies which are reactive as said may be prepared by known techniques, for example those disclosed in “Monoclonal Antibodies: A manual of techniques”, H Zola (CRC Press, 1988) and in “Monoclonal Hybridoma Antibodies: Techniques and Applications”, SGR Hurrell (CRC Press, 1982). Polyclonal antibodies may be produced which are polyspecific or monospecific. It is preferred that they are monospecific.

One embodiment provides an antibody reactive towards the polypeptide whose amino acid sequence is shown in FIG. 2 or natural variants thereof but not reactive towards RBP-1 or RBP-2 and other polypeptides encoded by polynucleotides identified by reference to FIG. 7.

A further embodiment provides an antibody reactive towards an epitope present in the polypeptide whose amino acid sequence is shown in FIG. 2 or natural variants thereof but which epitope is not present in RBP-1, RBP-2 and other polypeptides encoded by polynucleotides identified by reference to FIG. 7.

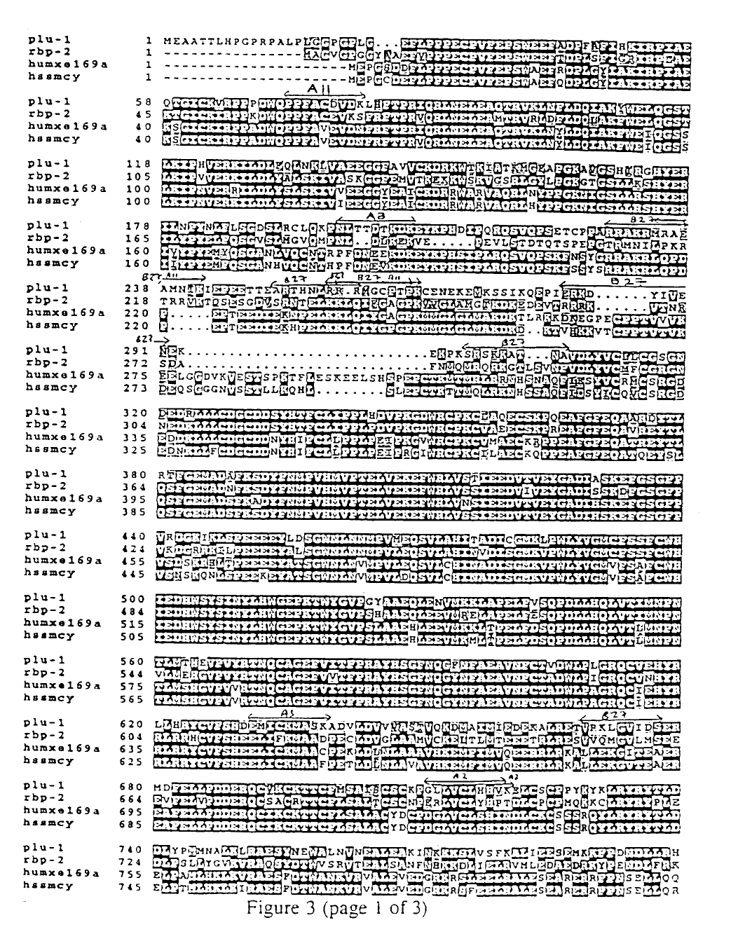

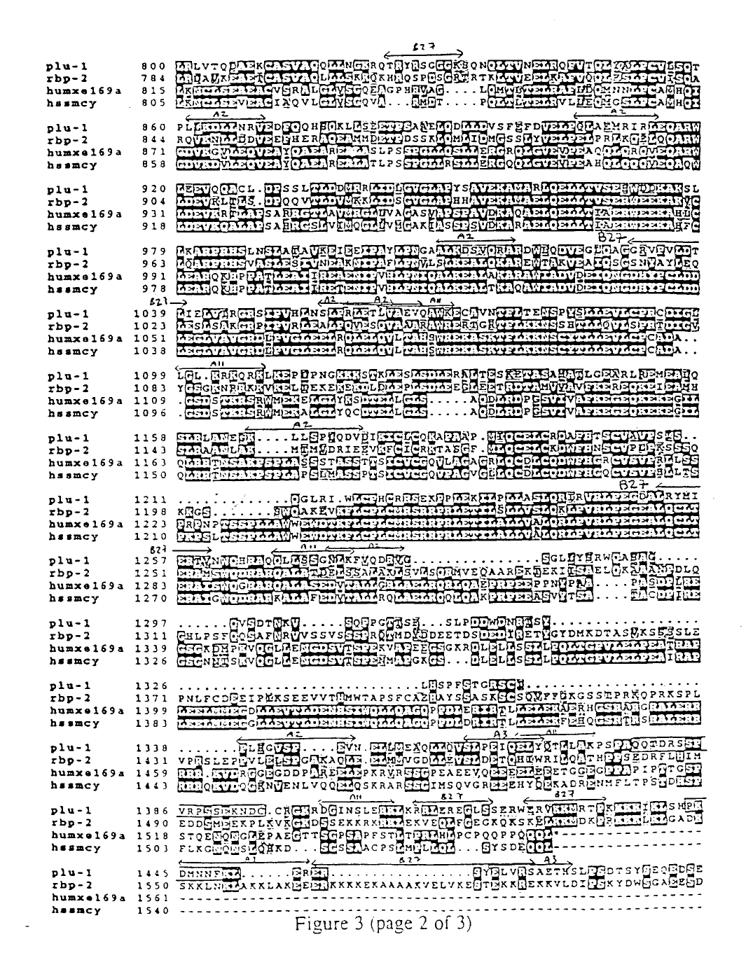

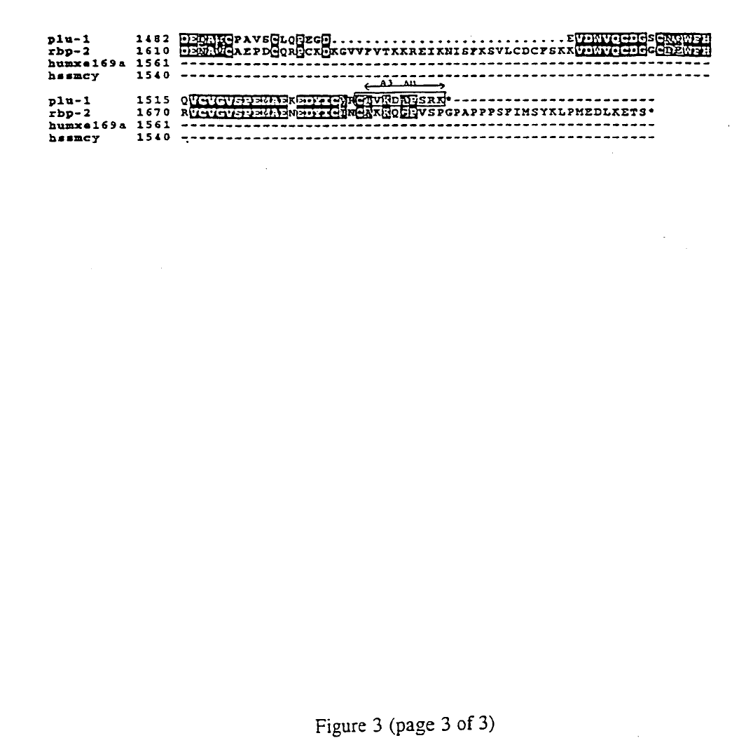

It is particularly preferred if the antibody is reactive towards a molecule comprising any one of the peptides: QQTDRSSPVRPSSEKNDC (amino acids 1378-1395); PKDMNNFKLERERSYELVR (amino acids 1443-1461); and CTVKDAPSRK (amino acids 1535-1544). These peptides are shown boxed in FIG. 2. FIG. 3 shows other peptides which may be used to distinguish plu-1 from its human homologue (boxed). Such antibodies may be made using these peptides as immunogens.

These peptides themselves may be useful for raising antibodies, but selective antibodies may be made using smaller fragments of these peptides which contain the region of difference between plu-i and RBP-1, RBP-2 or other polypeptides encoded by polynucleotides identified by reference to FIG. 7.

It may be convenient to raise antibodies using fragments of plu-I expressed as a fusion peptide.

Peptides in which one or more of the amino acid residues are chemically modified, before or after the peptide is synthesised, may be used providing that the function of the peptide, namely the production of specific antibodies in vivo, remains substantially unchanged. Such modifications include forming salts with acids or bases, especially physiologically acceptable organic or inorganic acids and bases, forming an ester or amide of a terminal carboxyl group, and attaching amino acid protecting groups such as N-t-butoxycarbonyl. Such modifications may protect the peptide from in vivo metabolism. The peptides may be present as single copies or as multiples, for example tandem repeats. Such tandem or multiple repeats may be sufficiently antigenic themselves to obviate the use of a carrier. It may be advantageous for the peptide to be formed as a loop, with the N-terminal and C-terminal ends joined together, or to add one or more Cys residues to an end to increase antigenicity and/or to allow disulphide bonds to be formed. If the peptide is covalently linked to a carrier, preferably a polypeptide, then the arrangement is preferably such that the peptide of the invention forms a loop.

According to current immunological theories, a carrier function should be present in any immunogenic formulation in order to stimulate, or enhance stimulation of, the immune system. It is thought that the best carriers embody (or, together with the antigen, create) a T-cell epitope. The peptides may be associated, for example by cross-linking, with a separate carrier, such as serum albumins, myoglobins, bacterial toxoids and keyhole limpet haemocyanin. More recently developed carriers which induce T-cell help in the immune response include the hepatitis-B core antigen (also called the nucleocapsid protein), presumed T-cell epitopes such as Thr-Ala-Ser-Gly-Val-Ala-Glu-Thr-Thr-Asn-Cys, beta-galactosidase and the 163-171 peptide of interleukin-1. The latter compound may variously be regarded as a carrier or as an adjuvant or as both. Alternatively, several copies of the same or different peptides of the invention may be cross-linked to one another; in this situation there is no separate carrier as such, but a carrier function may be provided by such cross-linking. Suitable cross-linking agents include those listed as such in the Sigma and Pierce catalogues, for example glutaraldehyde, carbodiimide and succinimidyl 4-(N-maleimidomethyl)cyclohexane-1-carboxylate, the latter agent exploiting the SH group on the C-terminal cysteine residue (if present).

If the peptide is prepared by expression of a suitable nucleotide sequence in a suitable host, then it may be advantageous to express the peptide as a fusion product with a peptide sequence which acts as a carrier. Kabigen's “Ecosec” system is an example of such an arrangement.

The peptide of the invention may be linked to other antigens to provide a dual effect.

A further aspect of the invention provides a method of making an antibody which is selectively reactive towards the polypeptide whose amino acid sequence is shown in FIG. 2 or a natural variant thereof, the method comprising the steps of, where appropriate, immunising an animal with a peptide which distinguishes plu-1 from other polypeptides and selecting an antibody which binds plu-1 but does not substantially bind other polypeptides. It is preferred if the antibodies do not substantially bind RBP-1 or RBP-2 or other polypeptides identified by reference to the polynucleotides referred to in FIG. 7. Suitable peptides are disclosed above.

It will be appreciated that, with the advancements in antibody technology, it may not be necessary to immunise an animal in order to produce an antibody. Synthetic systems, such as phage display libraries, may be used. The use of such systems is included in the methods of the invention.

Monoclonal antibodies which will bind to plu-1 antigens can be prepared. The antigen-binding portion may be a part of an antibody (for example a Fab fragment) or a synthetic antibody fragment (for example a single chain Fv fragment [ScFv]). Suitable monoclonal antibodies to selected antigens may be prepared by known techniques, for example those disclosed in “Monoclonal Antibodies: A manual of techniques”, H Zola (CRC Press, 1988) and in “Monoclonal Hybridoma Antibodies: Techniques and Applications”, J G R Hurrell (CRC Press, 1982).

Chimaeric antibodies are discussed by Neuberger et al (1988, 8th International Biotechnology Symposium Part 2, 792-799).

Suitably prepared non-human antibodies can be “humanized” in known ways, for example by inserting the CDR regions of mouse antibodies into the framework of human antibodies.

The variable heavy (V_{H}) and variable light (V_{L}) domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent parented antibody (Morrison et al (1984) Proc. Natl. Acad. Sci. USA 81, 6851-6855).

That antigenic specificity is conferred by variable domains and is independent of the constant domains is known from experiments involving the bacterial expression of antibody fragments, all containing one or more variable domains. These molecules include Fab-like molecules (Better et al (1988) Science 240, 1041); Fv molecules (Skerra et al (1988) Science 240, 1038); single-chain Fv (ScFv) molecules where the V_{H} and V_{L} partner domains are linked via a flexible oligopeptide (Bird et al (1988) Science 242, 423; Huston et al (1988) Proc. Natl. Acad. Sci. USA 85, 5879) and single domain antibodies (dAbs) comprising isolated V domains (Ward et al (1989) Nature 341, 544). A general review of the techniques involved in the synthesis of antibody fragments which retain their specific binding sites is to be found in Winter & Milstein (1991) Nature 349, 293-299.

By “ScFv molecules” we mean molecules wherein the V_{H} and V_{L} partner domains are linked via a flexible oligopeptide.

Fab, Fv, ScFv and dAb antibody fragments can all be made and expressed in and secreted from, for example, E. coli, thus allowing the facile production of large amounts of the said fragments.

Whole antibodies, and F(ab′)₂ fragments are “bivalent”. By “bivalent” we mean that the said antibodies and F(ab′)₂ fragments have two antigen combining sites. In contrast, Fab, Fv, ScFv and dAb fragments are monovalent, having only one antigen combining sites.

Before the present invention it was not possible to make use of the differences in amino acid sequence between RBP-1, RBP-2, other polypeptides and plu-1 in order to make antibodies which are useful in distinguishing plu-1 and RBP-1, RBP-2, and other polypeptides since it was not known that plu-1 and RBP-1, RBP-2 and other polypeptides had significant differences in structure or what those differences were. As is discussed in more detail below, such antibodies are useful in cancer diagnosis. It will also be appreciated that such antibodies which distinguish plu-1 and RBP-1, RBP-2 and other polypeptides are also useful research reagents. Suitably, the antibodies of the invention are detectably labelled, for example they may be labelled in such a way that they may be directly or indirectly detected. Conveniently, the antibodies are labelled with a radioactive moiety or a coloured moiety or a fluorescent moiety, or they may be linked to an enzyme. Typically, the enzyme is one which can convert a non-coloured (or non-fluorescent) substrate to a coloured (or fluorescent) product. The antibody may be labelled by biotin (or streptavidin) and then detected indirectly using streptavidin (or biotin) which has been labelled with a radioactive moiety or a coloured moiety or a fluorescent moiety, or the like or they may be linked to an enzyme of the type described above.

Anti-plu-1 antibodies or fragments or derivatives thereof such as humanised antibodies or ScFv fragments or dAbs or other fragments which retain antigen-binding specificity may be useful for imaging, such as imaging of tumours in the patient using, for example, radioimmunoscintigraphy. Conveniently, the antibodies or fragments or derivatives thereof are labelled with a moiety which allows detection. Suitably, the label is a radioactive moiety and, preferably, it contains ^{99m}Tc, or other suitable isotopes of technetium, or suitable isotopes of yttrium, indium, iodine or the like, all of which are well known in the art. Preferably, the antibody is a monoclonal antibody or fragment thereof.

Anti-plu-1 antibodies or fragments or derivatives thereof may be used therapeutically. For example, unconjugated antibodies or fragments or derivatives thereof may be used to induce an anti-idiotype response. Alternatively, antibodies or fragments or derivatives thereof may be conjugated to a moiety which is directly or indirectly cytotoxic. Directly cytotoxic agents include, for example, radioisotopes and toxins such as ricin; indirectly cytotoxic agents include, for example, enzymes which can convert a relatively non-toxic prodrug into a cytotoxic drug.

It is particularly preferred if peptides are made, based on the amino acid sequence of plu-1, which allow for specific antibodies to be made.

Thus, a further aspect of the invention provides a molecule which, following immunisation of an animal if appropriate, gives rise to antibodies which are reactive towards the polypeptide whose sequence is shown in FIG. 2 or natural variants thereof but not reactive towards other polypeptides such as RBP-1, RBP-2.

The molecule is preferably a peptide but may be any molecule which gives rise to the desired antibodies. The molecule, preferably a peptide, is conveniently formulated into an immunological composition using methods well known in the art.

The peptides disclosed above form part of these aspects of the invention.

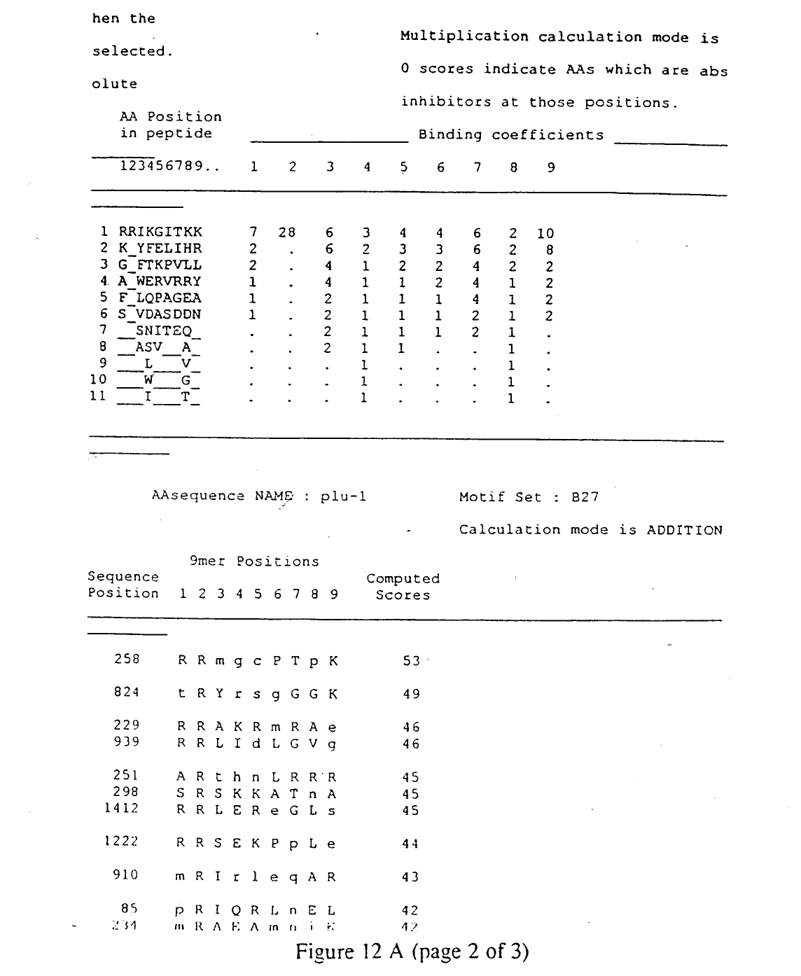

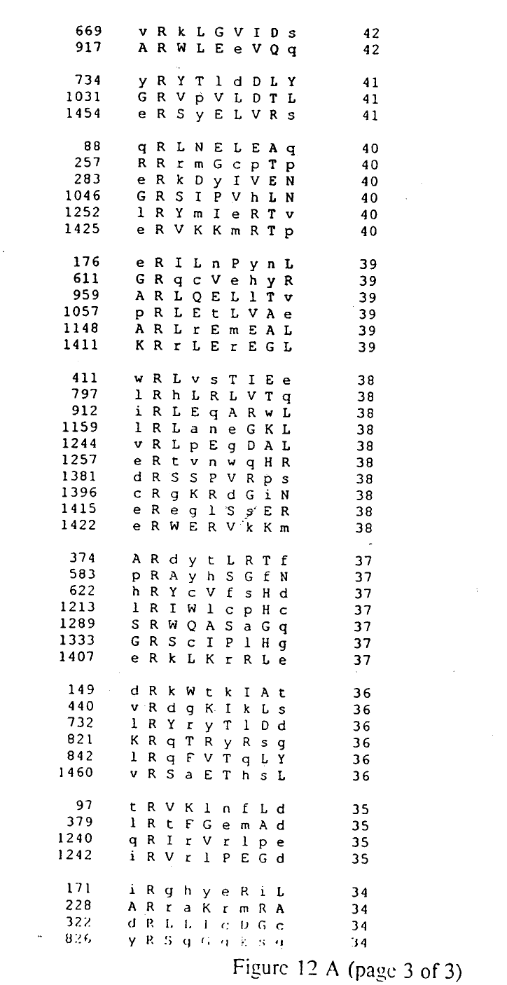

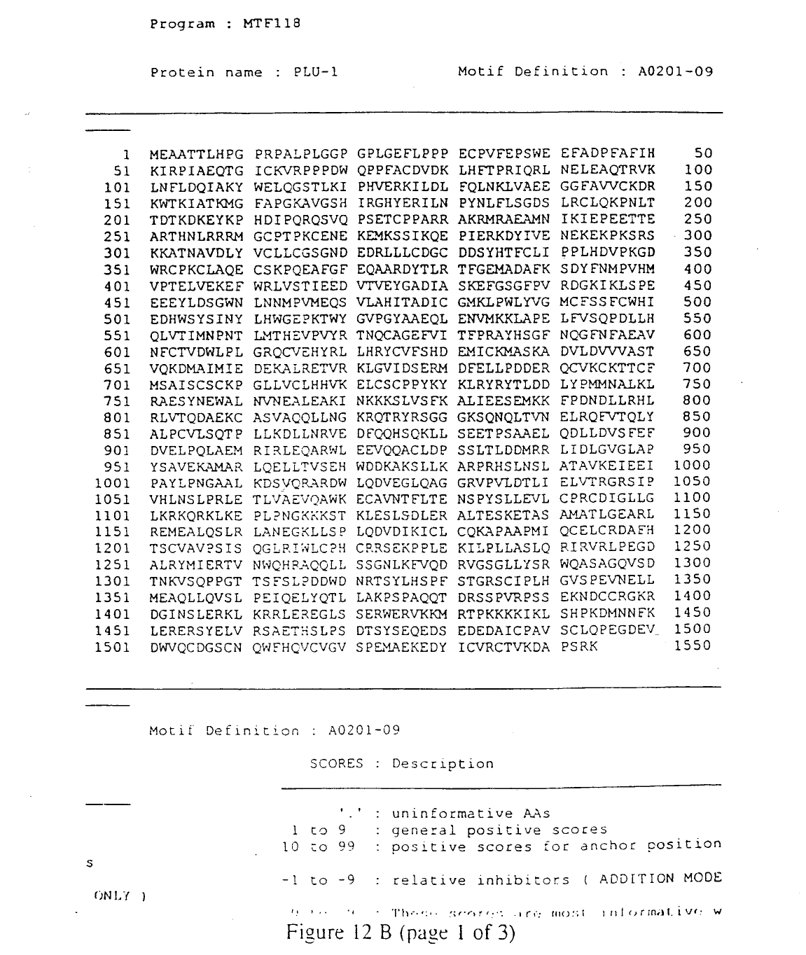

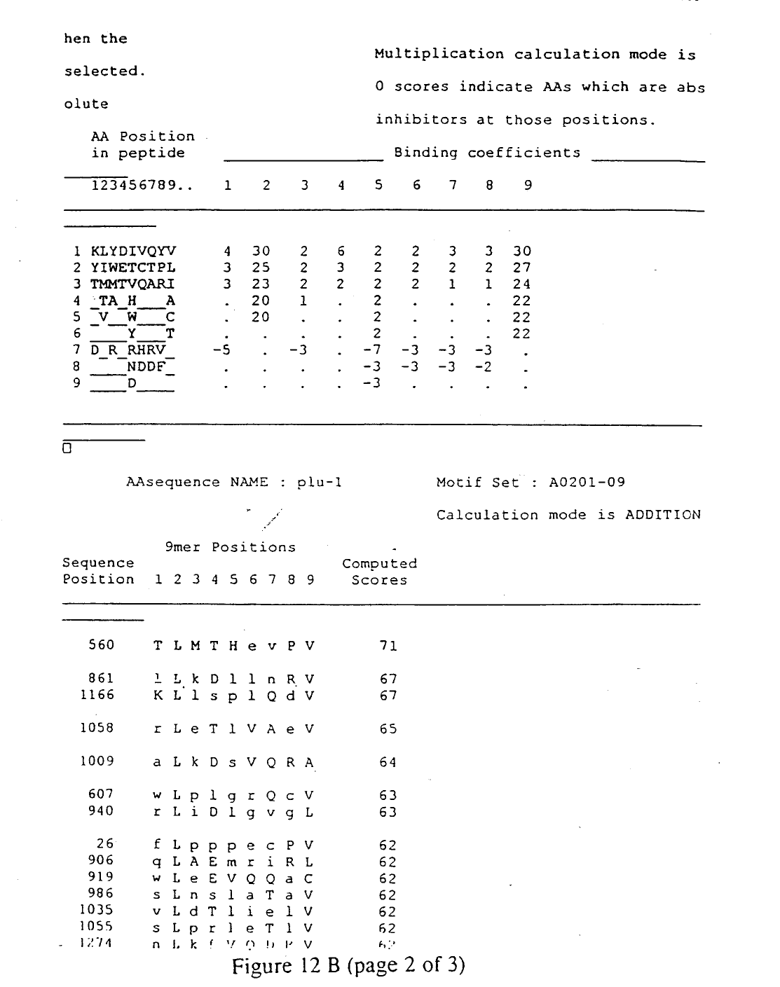

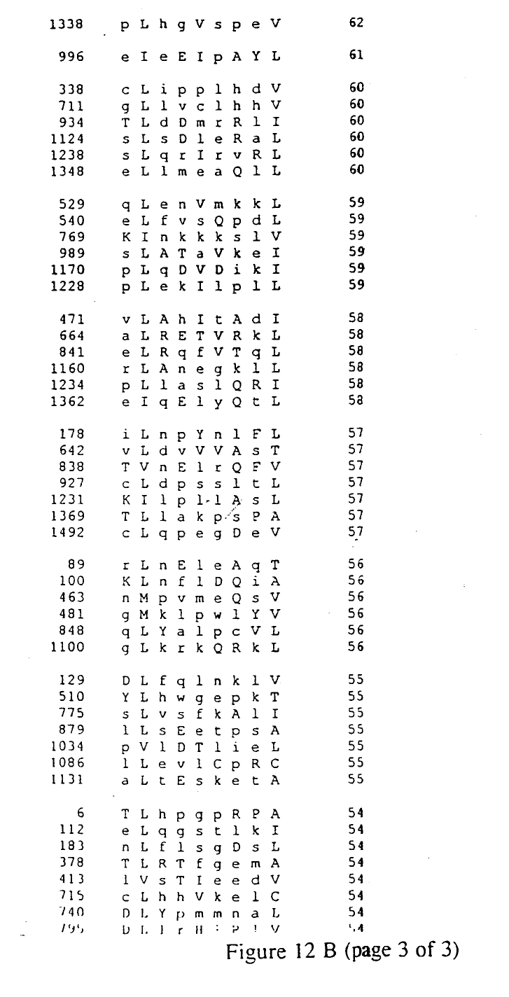

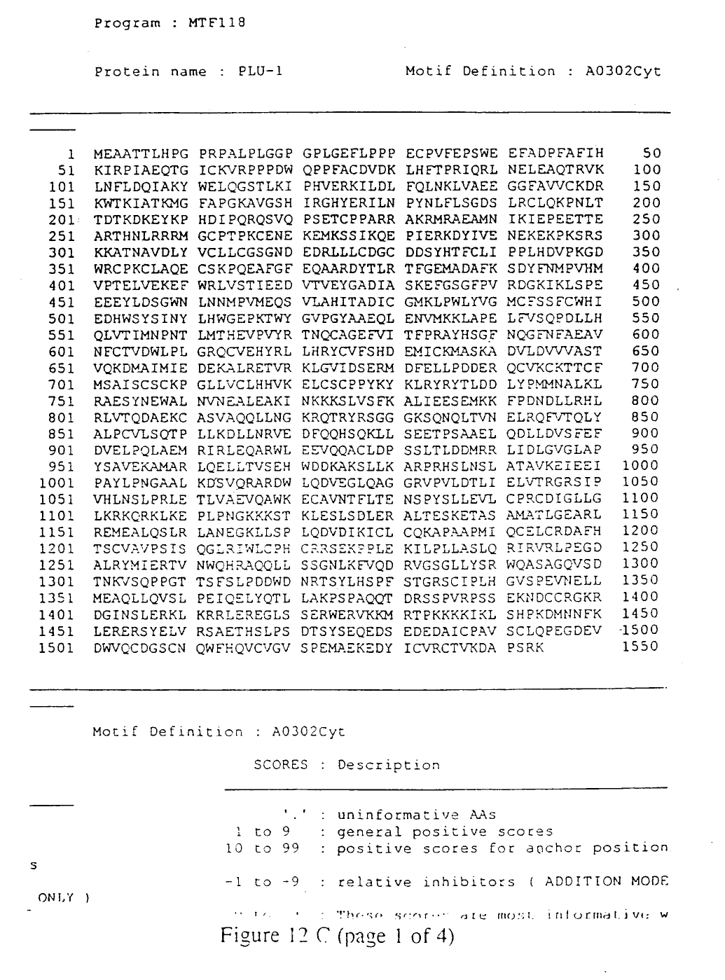

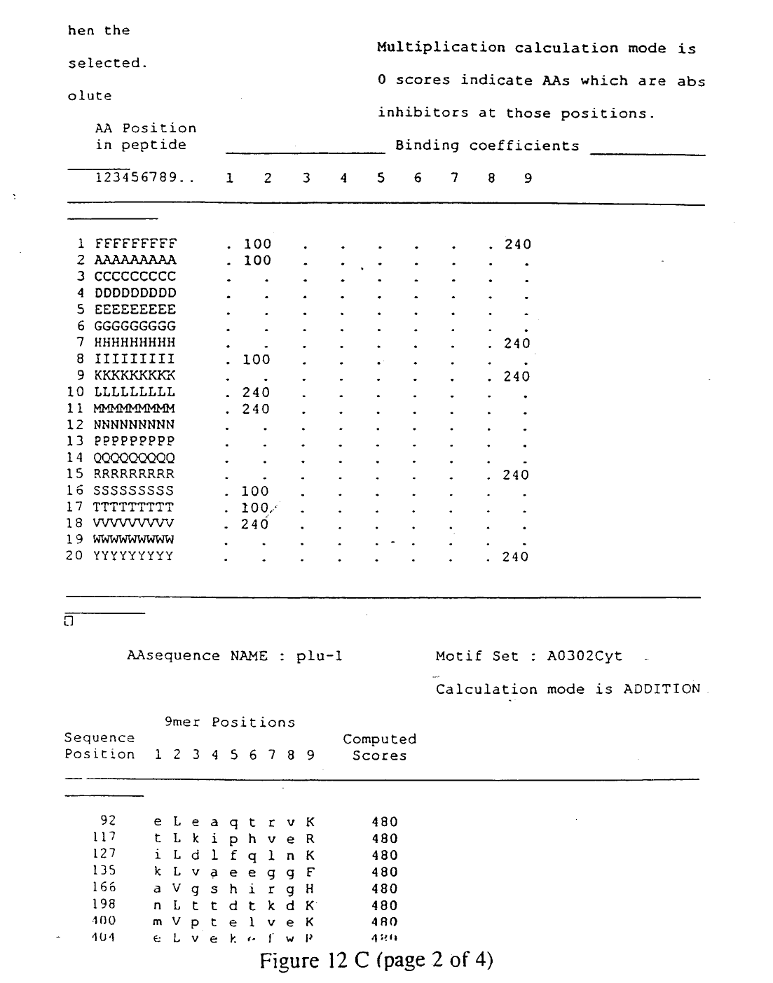

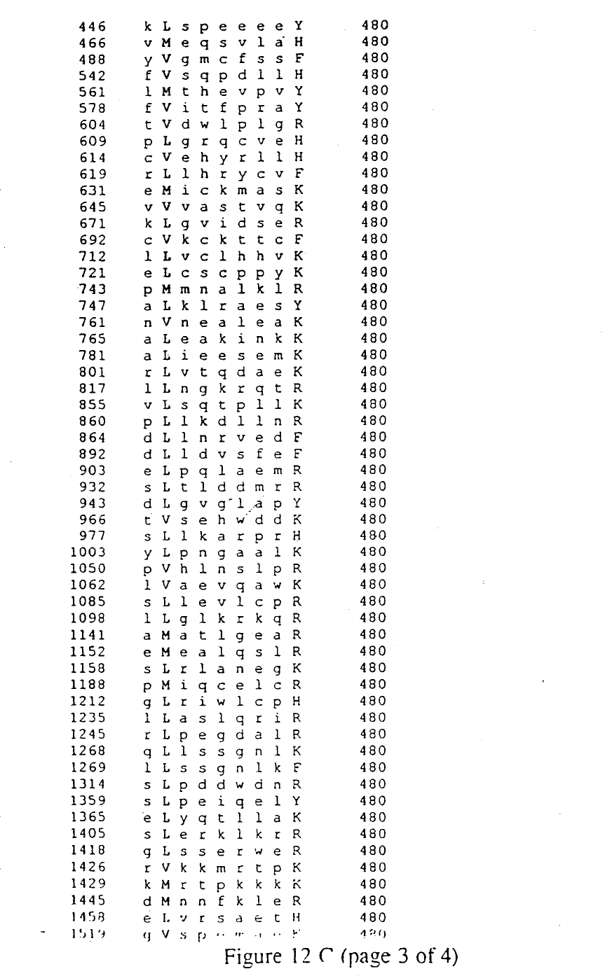

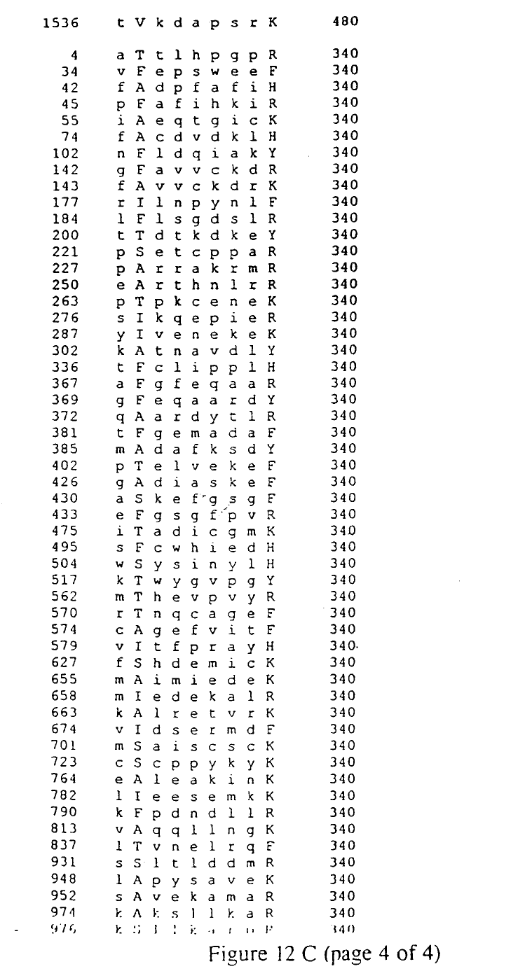

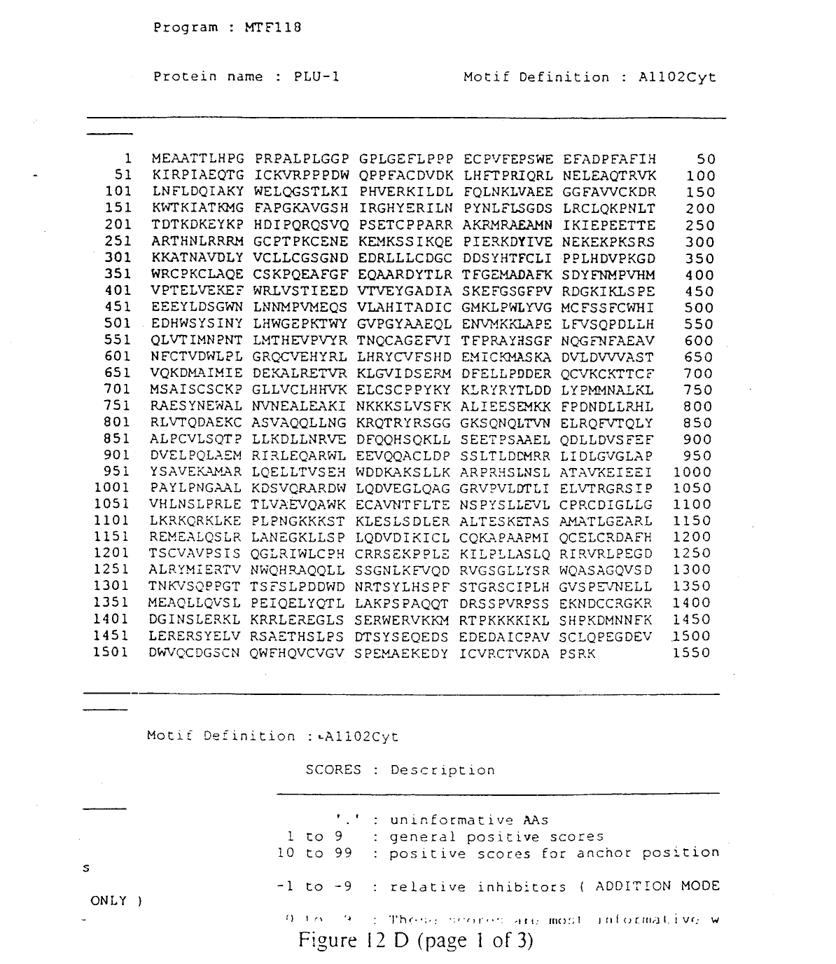

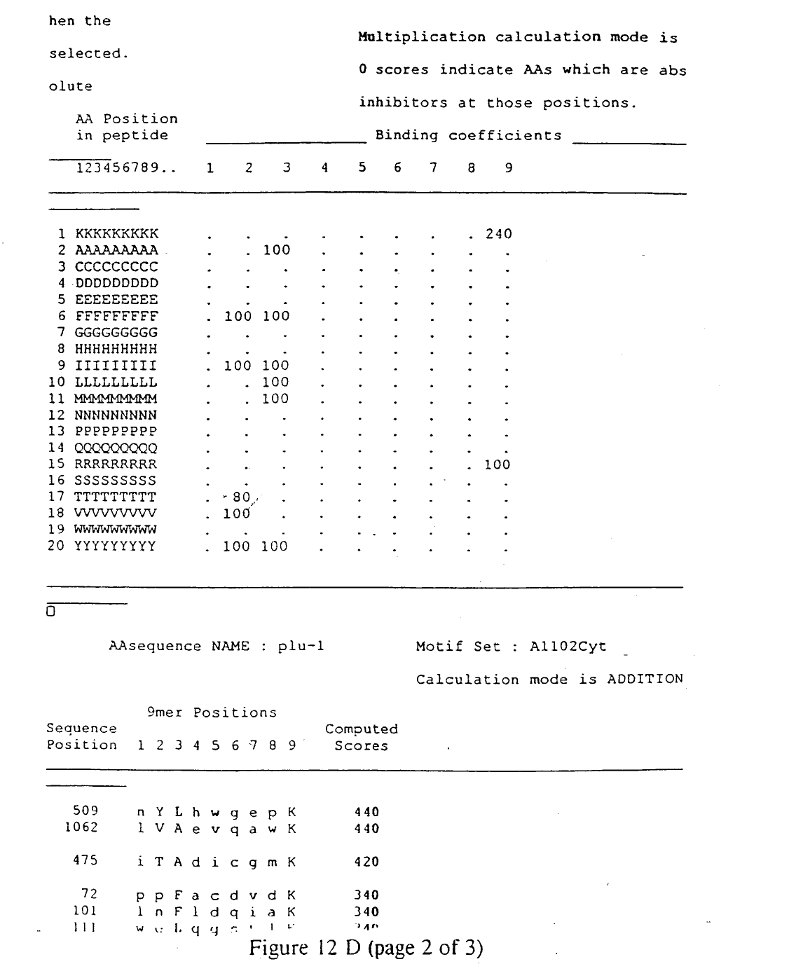

Peptides derived from plu-1 are not only useful for raising antibodies but are also useful for binding MHC (HLA) molecules. Preferred peptides are shown in FIG. 12. FIG. 12 shows searches for HLA-B27, HLA-A2, HLA-A3, and HLA-A 11 MHC epitopes. Searches for peptides predicted to bind other class I epitopes may be performed using computer program. For example, a suitable program is available on the World Wide Web at http://bimas.dcrt.gov/molbio/hla_bind/, and is described in Parker et al (1994) J. Immunol. 152, 163. The frequencies of the HLA antigens in Caucasian populations are: 6.7%, 49.4%, 24.7% and 12.2%, respectively (Baur et al (1984) “Population analysis on the basis of deduced haplotypes from random families”. In “Histocompatibility Testing”, eds, Albert, Baur & Mayr, Springer Verlag, Berlin Suitable Class I epitopes are shown in FIG. 3. Their positions (starting position in the amino acid sequence) are listed, with those particularly preferred marked with an asterisk (*): HLA-B27:229, 234, 251, 257, 258, 283, 298, 669, 824, 1031, 1252, 1412, 1425 and 1454; HLA-A2:711*, 861, 906*, 1009, 1055, 1058*, 1166, 1274, 1338*; HLA-A3:198, 631, 712, 1359, 1445, 1458 and 1536; HLA-A11:72, 234, 258, 1062, 1099, 1268, 1365, 1401 and 1536. The peptides are preferably nonamers. The peptides marked (*) are distinguished from RBP-2. These peptides, and the peptides listed in FIG. 12, are believed to be particularly useful in cancer vaccines or in other cancer immunotherapeutic approaches.

It may be useful to introduce alanine substitutions into the peptides in order to improve binding affinity without abrogating peptide-specific CTL recognition as is described in Collins et al (1989) J. Immunol. 162, 331-337.

Peptides may be synthesised by the Fmoc-polyamide mode of solid-phase peptide synthesis as disclosed by Lu et al (1981) J. Org. Chem. 46, 3433 and references therein. Temporary N-amino group protection is afforded by the 9-fluorenylmethyloxycarbonyl (Fmoc) group. Repetitive cleavage of this highly base-labile protecting group is effected using 20% piperidine in N,N-dimethylformamide. Side-chain functionalities may be protected as their butyl ethers (in the case of serine threonine and tyrosine), butyl esters (in the case of glutamic acid and aspartic acid), butyloxycarbonyl derivative (in the case of lysine and histidine), trityl derivative (in the case of cysteine) and 4-methoxy-2,3,6-trimethylbenzenesulphonyl derivative (in the case of arginine). Where glutamine or asparagine are C-terminal residues, use is made of the 4,4′-dimethoxybenzhydryl group for protection of the side chain amido functionalities. The solid-phase support is based on a polydimethyl-acrylamide polymer constituted from the three monomers dimethylacrylamide (backbone-monomer), bisacryloylethylene diamine (cross linker) and acryloylsarcosine methyl ester (functionalising agent). The peptide-to-resin cleavable linked agent used is the acid-labile 4-hydroxymethyl-phenoxyacetic acid derivative. All amino acid derivatives are added as their preformed symmetrical anhydride derivatives with the exception of asparagine and glutamine, which are added using a reversed N,N-dicyclohexyl-carbodiimide/1-hydroxybenzotriazole mediated coupling procedure. All coupling and deprotection reactions are monitored using ninhydrin, trinitrobenzene sulphonic acid or isotin test procedures. Upon completion of synthesis, peptides are cleaved from the resin support with concomitant removal of side-chain protecting groups by treatment with 95% trifluoroacetic acid containing a 50% scavenger mix. Scavengers commonly used are ethanedithiol, phenol, anisole and water, the exact choice depending on the constituent amino acids of the peptide being synthesised. Trifluoroacetic acid is removed by evaporation in vacuo, with subsequent trituration with diethyl ether affording the crude peptide. Any scavengers present are removed by a simple extraction procedure which on lyophilisation of the aqueous phase affords the crude peptide free of scavengers. Reagents for peptide synthesis are generally available from Calbiochem-Novabiochem (UK) Ltd, Nottingham NG72QJ, UK. Purification may be effected by any one, or a combination of, techniques such as size exclusion chromatography, ion-exchange chromatography and (principally) reverse-phase high performance liquid chromatography. Analysis of peptides may be carried out using thin layer chromatography, reverse-phase high performance liquid chromatography, amino-acid analysis after acid hydrolysis and by fast atom bombardment (FAB) mass spectrometric analysis.

By “peptides” we include compounds which function in the same way as peptides in raising an immune response. For example, the term “peptide” specifically includes molecules which may have the same side chains of amino acids in the peptide but wherein, for example, the peptide linkage has been replaced by another linkage which, whilst having the same geometry as a peptide bond, is less susceptible to degradation. Thus, peptidomimetics are included in the definition of “peptides”.

By “peptide” we also include not only molecules in which amino acid residues are joined by peptide (—CO—NH—) linkages but also molecules in which the peptide bond is reversed. Such retro-inverso peptidomimetics may be made using methods known in the art, for example such as those described in Meziere et al (1997) J. Immunol. 159, 3230-3237, incorporated herein by reference. This approach involves making pseudopeptides containing changes involving the backbone, and not the orientation of side chains. Meziere et al (1997) show that, at least for MHC class II and T helper cell responses, these pseudopeptides are useful. Retro-inverse peptides, which contain NH—CO bonds instead of CO—NH peptide bonds, are much more resistant to proteolysis.

Similarly, the peptide bond may be dispensed with altogether provided that an appropriate linker moiety which retains the spacing between the Cα atoms of the amino acid residues is used; it is particularly preferred if the linker moiety has substantially the same charge distribution and substantially the same planarity of a peptide bond.

It will be appreciated that the peptide may conveniently be blocked at its N- or C-terminus so as to help reduce susceptibility to exoproteolytic digestion.

It is now possible to make polynucleotides which can distinguish plu-1 mRNA, cDNA or gene and other RNAs, cDNAs and genes and such polynucleotides are believed to be useful in the diagnosis and prognosis of cancer. In particular, the polynucleotide distinguishes plu-1 mRNA, cDNA or gene from RBP-1 or RBP-2 RNAs, cDNAs or genes.

A further aspect of the invention provides a polynucleotide which distinguishes a polynucleotide which encodes the polypeptide whose sequence is shown in FIG. 2 or a natural variant thereof and which encodes another polypeptide such as RBP-1, RBP-2 or polypeptides which are encoded by polynucleotides identified by reference to FIG. 7.

A yet still further aspect of the invention provides a polynucleotide which hybridises to a polynucleotide which encodes the polypeptide whose sequence is shown in FIG. 2 or a natural variant thereof but not to a polynucleotide which encodes another polypeptide such as RBP-1, RBP-2 or polypeptides which are encoded by polynucleotides identified by reference to FIG. 7.

Such polynucleotides can be designed by reference to FIGS. 1 and 2 and the known sequence of RBP-1, RBP-2 and the Figures of this patent application, in particular FIG. 7, and may be synthesised by well known methods such as by chemical synthesis or by using specific primers and template, a DNA amplification technique such as the polymerase chain reaction. The polynucleotide may be any polynucleotide, whether DNA or RNA or a synthetic nucleic acid such as a peptide nucleic acid, provided that it can distinguish polynucleotides which encode plu-1 and polynucleotides, which encode other polypeptides as said. It is particularly preferred if the polynucleotide is an oligonucleotide which can serve as a hybridisation probe or as a primer for a nucleic acid amplification system. Thus, the polynucleotide of this aspect of the invention may be an oligonucleotide of at least 10 nucleotides in length, more preferably at least 14 nucleotides in length and still more preferably at least 18 nucleotides in length.

It is particularly preferred that the polynucleotide hybridises to a mRNA (or cDNA) which encodes plu-1 but does not hybridise to another mRNA (or cDNA), for example, one which encodes RBP-1 or RBP-2.

Preferably, the polynucleotides of the invention are detectably labelled. For example, they may be labelled in such a way that they may be directly or indirectly detected. Conveniently, the polynucleotides are labelled with a radioactive moiety or a coloured moiety or a fluorescent moiety or some other suitable detectable moiety such as digoxygenin and luminescent or chemiluminescent moieties. The polynucleotides may be linked to an enzyme, or they may be linked to biotin (or streptavidin) and detected in a similar way as described for antibodies of the invention. Also preferably the polynucleotides of the invention may be bound to a solid support (including arrays, beads, magnetic beads, sample containers and the like). The polynucleotides of the invention may also incorporate a “tag” nucleotide sequence which tag sequence can subsequently be recognised by a further nucleic acid probe. Suitable labels or tags may also be used for the selective capture of the hybridised (or non-hybridised) polynucleotide using methods well known in the art.

A further aspect of the invention provides a method for determining the susceptibility of a patient to cancer comprising the steps of (i) obtaining a sample containing nucleic acid from the patient; and (ii) contacting the said nucleic acid with a nucleic acid which hybridises selectively to plu-1 nucleic acid.

A still further aspect of the invention provides a method of diagnosing cancer in a patient comprising the steps of (i) obtaining a sample containing nucleic acid from the patient; and (ii) contacting the said nucleic acid with a nucleic acid which hybridises selectively to plu-1 nucleic acid.

A yet still further aspect of the invention provides a method of predicting the relative prospects of a particular outcome of a cancer in a patient comprising the steps of (i) obtaining a sample containing nucleic acid from the patient; and (ii) contacting the said nucleic acid with a nucleic acid which hybridises selectively to plu-1 nucleic acid.

Preferably, the nucleic acid in the sample is mRNA.

It will be appreciated that detecting the presence of an increased level of plu-1 mRNA in a cell compared to the level present in a normal (non-tumorigenic) cell may suggest that the patient will benefit from a particular form of treatment, such as treatment with a plu-1 tumour vaccine as herein disclosed.

Transcription of plu-1 seems to be substantially completely repressed in normal adult tissue with the exception of the testis and with some expression in placenta, ovary and tonsil. This repression is absent in breast tumours, causing plu-1 to be expressed. The derepression of plu-1 transcription may be caused by methylation defects in cancer cells. Increased plu-1 mRNA in a sample compared to that found in a normal (non-tumorigenic) tissue sample is indicative of carcinogenesis. Typically, the level in a tumorigenic sample is at least 2-fold, preferably at least 5-fold and more preferably at least 10-fold more in a tumorigenic sample compared to a known, normal (non-tumorigenic) tissue sample.

It may also be advantageous to measure the presence (tumour) versus absence (normal) of plu-1 mRNA in some circumstances, such as when assessing breast tissue.

By “selectively hybridising” is meant that the nucleic acid has sufficient nucleotide sequence similarity with the said plu-1 nucleic acid that it can hybridise under moderately or highly stringent conditions. As is well known in the art, the stringency of nucleic acid hybridization depends on factors such as length of nucleic acid over which hybridisation occurs, degree of identity of the hybridizing sequences and on factors such as temperature, ionic strength and CG or AT content of the sequence. Thus, any nucleic acid which is capable of selectively hybridising as said is useful in the practice of the invention.

Nucleic acids which can selectively hybridise to the said plu-1 nucleic acid (eg mRNA) include nucleic acids which have >95% sequence identity, preferably those with >98%, more preferably those with >99% sequence identity, over at least a portion of the nucleic acid with the said nucleic acid (eg MRNA).

It is preferred if the nucleic acid which hybridises selectively to plu-1 nucleic acid does not hybridise to any other nucleic acid (eg mRNA), such as RBP-1 nucleic acid (eg mRNA) or RBP-2 nucleic acid (eg mRNA).

Typical moderately or highly stringent hybridisation conditions which lead to selective hybridisation are known in the art, for example those described in Molecular Cloning, a laboratory manual, 2nd edition, Sambrook et al (eds), Cold Spring Harbor Laboratory Press, Cold Spring Harbor, N.Y., USA, incorporated herein by reference.

An example of a typical hybridisation solution when a nucleic acid is immobilised on a nylon membrane and the probe nucleic acid is >500 bases or base pairs is:

6×SSC (saline sodium citrate)

0.5% sodium dodecyl sulphate (SDS)

100 μg/ml denatured, fragmented salmon sperm DNA

The hybridisation is performed at 68° C. The nylon membrane, with the nucleic acid immobilised, may be washed at 68° C. in 1×SSC or, for high stringency, 0.1×SSC.

20×SSC may be prepared in the following way. Dissolve 175.3 g of NaCl and 88.2 g of sodium citrate in 800 ml of H₂O. Adjust the pH to 7.0 with a few drops of a 10 N solution of NaOH. Adjust the volume to 1 litre with H₂O. Dispense into aliquots. Sterilize by autoclaving.

The assay of plu-1 mRNA may be by an indirect means.

An example of a typical hybridisation solution when a nucleic acid is immobilised on a nylon membrane and the probe is an oligonucleotide of between 15 and 50 bases is:

3.0 M trimethylammonium chloride (TMACl)

0.01 M sodium phosphate (pH 6.8)

1 mm EDTA (pH 7.6)

0.5% SDS

100 μg/ml denatured, fragmented salmon sperm DNA

0.1% nonfat dried milk

The optimal temperature for hybridization is usually chosen to be 5° C. below the Tᵢ for the given chain length. Tᵢ is the irreversible melting temperature of the hybrid formed between the probe and its target sequence. Jacobs et al (1988) Nucl. Acids Res. 16, 4637 discusses the determination of Tᵢs. The recommended hybridization temperature for 17-mers in 3 M TMACl is 48-50° C.; for 19-mers, it is 55-57° C.; and for 20-mers, it is 58-66° C.

By “nucleic acid which selectively hybridises” is also included nucleic acids which will amplify DNA (for example, copied from plu-1 mRNA by, for example, reverse transcription) by any of the well known amplification systems such as those described in more detail below, in particular the polymerase chain reaction (PCR). Suitable conditions for PCR amplification include amplification in a suitable 1× amplification buffer:

10× amplification buffer is 500 mM KCl; 100 mM Tris.Cl (pH 8.3 at room temperature); 15 mM MgCl₂; 0.1% gelatin.

A suitable denaturing, agent or procedure (such as heating to 95° C.) is used in order to separate the strands of double-stranded DNA.

Suitably, the annealing part of the amplification is between 37° C. and 60° C., preferably 50° C.

Although the nucleic acid which is useful in the methods of the invention may be RNA or DNA, DNA is preferred. Although the nucleic acid which is useful in the methods of the invention may be double-stranded or single-stranded, single-stranded nucleic acid is preferred under some circumstances such as in nucleic acid amplification reactions.

As is described more fully below, single-stranded DNA primers, suitable for use in a polymerase chain reaction, are particularly preferred.

The nucleic acid for use in the methods of the invention is a nucleic acid which hybridises to plu-1 nucleic acid (eg MRNA). cDNAs derivable from the plu-1 mRNA are preferred nucleic acids for use in the methods of the invention.

The plu-1 gene and plu-l cDNA are similar to, but distinct from, the RBP-1 gene and cDNA, and the RBP-2 gene and cDNA and certain other cDNA portions described in the application. Preferred nucleic acids for use in the invention are those that selectively hybridise to the plu-1 nucleic acid (eg mRNA) and do not hybridise to other nucleic acids such as RBP-1 mRNA and RBP-2 MRNA. Such selectively hybridising nucleic acids can be readily obtained, for example, by reference to whether or not they hybridise to plu-1 cDNA as shown in FIG. 1 and by reference to whether or not they hybridise to known sequences, such as the RBP-1 and RBP-2 sequences.

The methods may be suitable in respect of any cancer but it is preferred if the cancer is cancer of the ovary or breast. It is preferred if the cancer is not testicular cancer or colon cancer. The methods are most suitable in respect of breast cancer. It will be appreciated that the methods of the invention include methods of prognosis and methods which aid diagnosis. It will also be appreciated that the methods of the invention are useful to the physician or surgeon in determining a course of management or treatment of the patient.

The diagnostic and prognostic methods of the invention are particularly suited to female patients.

Although it is believed that any sample containing nucleic acid derived from the patient may be useful in the methods of the invention, it is preferred if the nucleic acid is derived from a sample of the tissue in which cancer is suspected or in which cancer may be or has been found. For example, if the tissue in which cancer is suspected or in which cancer may be or has been found is breast, it is preferred if the sample containing nucleic acid is derived from the breast of the patient. Breast samples may be obtained by excision, “true cut” biopsies, needle biopsy, nipple aspirate or image-guided biopsy.

The sample may be directly derived from the patient, for example, by biopsy of the tissue, or it may be derived from the patient from a site remote from the tissue, for example because cells from the tissue have migrated from the tissue to other parts of the body. Alternatively, the sample may be indirectly derived from the patient in the sense that, for example, the tissue or cells therefrom may be cultivated in vitro, or cultivated in a xenograft model; or the nucleic acid sample may be one which has been replicated (whether in vitro or in vivo) from nucleic acid from the original source from the patient. Thus, although the nucleic acid derived from the patient may have been physically within the patient, it may alternatively have been copied from nucleic acid which was physically within the patient. The tumour tissue may be taken from the primary tumour or from metastases. The sample may be lymph nodes, lymph or blood and the spread of disease detected.

Conveniently, the nucleic acid capable of selectively hybridising to the said plu-1 mRNA and which is used in the methods of the invention further comprises a detectable label.

By “detectable label” is included any convenient radioactive label such as ³²P, ³³P or ³⁵S which can readily be incorporated into a nucleic acid molecule using well known methods; any convenient fluorescent or chemiluminescent label which can readily be incorporated into a nucleic acid is also included. In addition the term “detectable label” also includes a moiety which can be detected by virtue of binding to another moiety (such as biotin which can be detected by binding to streptavidin); and a moiety, such as an enzyme, which can be detected by virtue of its ability to convert a colourless compound into a coloured compound, or vice versa (for example, alkaline phosphatase can convert colourless o nitrophenylphosphate into coloured o-nitrophenol). Conveniently, the nucleic acid probe may occupy a certain position in a fixed assay and whether the nucleic acid hybridises to the said region of human DNA can be determined by reference to the position of hybridisation in the fixed assay. The detectable label may also be a fluorophore-quencher pair as described in Tyagi & Kramer (1996) Nature Biotechnology 14, 303-308.

Other types of labels and tags are disclosed above. The nucleic acid may be branched nucleic acid (see Urdea et al (1991) Nucl. Acids Symposium Series 24, 197-200).

It will be appreciated that the aforementioned methods may be used for presymptomatic screening of a patient who is in a risk group for cancer. High risk patients for screening include patients over 50 years of age or patients who carry a gene resulting in increased susceptibility (eg predisposing versions of BRCA1, BRCA2 or p53); patients with a family history of breast/ovarian cancer; patients with affected siblings; nulliparous women; and women who have a long interval between menarche and menopause. Similarly, the methods may be used for the pathological classification of tumours such as breast tumours.

As is described in more detail in the Examples, plu-1 mRNA is absent or weakly expressed in benign breast tumours. There is some expression in ductal carcinoma in situ (DCIS) which is an early stage of carcinogenesis. Increased expression of plu-1 mRNA is seen in invasive breast carcinomas. There is some expression of plu-1 mRNA in ovarian tumours, and some plu-1 expression is seen in foetal tissue, consistent with a postulated role in development.

Conveniently, in the methods of the invention the nucleic acid which is capable of the said selective hybridisation (whether labelled with a detectable label or not) is contacted with nucleic acid (eg mRNA) derived from the patient under hybridising conditions. Suitable hybridising conditions include those described above.

The presence of a complex which is selectively formed by the nucleic acid hybridising to plu-1 mRNA may be detected, for example the complex may be a DNA:RNA hybrid which can be detected using antibodies. Alternatively, the complex formed upon hybridisation may be a substrate for an enzymatic reaction the product of which may be detected (suitable enzymes include polymerases, ligases and endonucleases).

It is preferred that if the sample containing nucleic acid (eg mRNA) derived from the patient is not a substantially pure sample of the tissue or cell type in question that the sample is enriched for the said tissue or cells. For example, enrichment for breast cells in a sample such as a blood sample may be achieved using, for example, cell sorting methods such as fluorescent activated cell sorting (FACS) using a breast cell-selective antibody, or at least an antibody which is selective for an epithelial cell. For example, anti-MUC1 antibodies such as HMFG-1 and HMFG-2 may be used (Taylor-Papadimitriou et al (1986) J. Exp. Pathol. 2, 247-260); other anti-MUC1 antibodies which may be useful are described in Cao et al (1998) Tumour Biol. 19, (Suppl 1), 88-99. The source of the said sample also includes biopsy material as discussed above and tumour samples, also including fixed paraffin mounted specimens as well as fresh or frozen tissue. The nucleic acid sample from the patient may be processed prior to contact with the nucleic acid which selectively hybridises to plu-1 mRNA. For example, the nucleic acid sample from the patient may be treated by selective amplification, reverse transcription, immobilisation (such as sequence specific immobilisation), or incorporation of a detectable marker.

It will be appreciated that plu-1 mRNA may be identified by reverse-transcriptase polymerase chain reaction (RT-PCR) using methods well known in the art.

Primers which are suitable for use in a polymerase chain reaction (PCR; Saiki et al (1988) Science 239, 487491) are preferred. Suitable PCR primers may have the following properties:

It is well known that the sequence at the 5′ end of the oligonucleotide need not match the target sequence to be amplified.

It is usual that the PCR primers do not contain any complementary structures with each other longer than 2 bases, especially at their 3′ ends, as this feature may promote the formation of an artifactual product called “primer dimer”. When the 3′ ends of the two primers hybridize, they form a “primed template” complex, and primer extension results in a short duplex product called “primer dimer”.

Internal secondary structure should be avoided in primers. For symmetric PCR, a 40-60% G+C content is often recommended for both primers, with no long stretches of any one base. The classical melting temperature calculations used in conjunction with DNA probe hybridization studies often predict that a given primer should anneal at a specific temperature or that the 72° C. extension temperature will dissociate the primer/template hybrid prematurely. In practice, the hybrids are more effective in the PCR process than generally predicted by simple Tm calculations.

Optimum annealing temperatures may be determined empirically and may be higher than predicted. Taq DNA polymerase does have activity in the 37-55° C. region, so primer extension will occur during the annealing step and the hybrid will be stabilized. The concentrations of the primers are equal in conventional (symmetric) PCR and, typically, within 0.1- to 1-μM range.

Any of the nucleic acid amplification protocols can be used in the method of the invention including the polymerase chain reaction, QB replicase and ligase chain reaction. Also, NASBA (nucleic acid sequence based amplification), also called 3SR, can be used as described in Compton (1991) Nature 350, 91-92 and AIDS (1993), Vol 7 (Suppl 2), S108 or SDA (strand displacement amplification) can be used as described in Walker et al (1992) Nucl. Acids Res. 20, 1691-1696. The polymerase chain reaction is particularly preferred because of its simplicity.

When a pair of suitable nucleic acids of the invention are used in a PCR it is convenient to detect the product by gel electrophoresis and ethidiun bromide staining. As an alternative to detecting the product of DNA amplification using agarose gel electrophoresis and ethidium bromide staining of the DNA, it is convenient to use a labelled oligonucleotide capable of hybridising to the amplified DNA as a probe. When the amplification is by a PCR the oligonucleotide probe hybridises to the interprimer sequence as defined by the two primers. The oligonucleotide probe is preferably between 10 and 50-nucleotides long, more preferably between 15 and 30 nucleotides long. The probe may be labelled with a radionuclide such as ³²P, ³³P and ³⁵S using standard techniques, or may be labelled with a fluorescent dye. When the oligonucleotide probe is fluorescently labelled, the amplified DNA product may be detected in solution (see for example Balaguer et al (1991) “Quantification of DNA sequences obtained by polymerase chain reaction using a bioluminescence adsorbent” Anal. Biochem. 195, 105-110 and DiCesare et al (1993) “A high-sensitivity electrochemiluminescence-based detection system for automated PCR product quantitation” BioTechniques 15, 152-157.

Amplification products can also be detected using a probe which may have a fluorophore-quencher pair or may be attached to a solid support or may have a biotin tag or they may be detected using a combination of a capture probe and a detector probe.

Fluorophore-quencher pairs are particularly suited to quantitative measurements of PCR reactions (eg RT-PCR). Fluorescence polarisation using a suitable probe may also be used to detect PCR products.

Oligonucleotide primers can be synthesised using methods well known in the art, for example using solid-phase phosphoramidite chemistry.

The present invention provides the use of a nucleic acid which selectively hybridises to plu-1 nucleic acid (eg mRNA) in a method of diagnosing cancer or prognosing cancer or determining susceptibility to cancer; or in the manufacture of a reagent for carrying out these methods.

Other methods of detecting MRNA levels are included.

Methods for determining the relative amount of plu-1 MRNA include: in situ hybridisation (In Situ Hybridization Protocols. Methods in Molecular Biology Volume 33. Edited by K H A Choo. 1994, Humana Press Inc (Totowa, N.J., USA) pp 480p and In Situ Hybridization: A Practical Approach. Edited by D G Wilkinson. 1992, Oxford University Press, Oxford, pp 163), in situ amplification, northerns, nuclease protection, probe arrays, and amplification based systems;

The mRNA may be amplified prior to or during detection and quantitation ‘Real time’ amplification methods wherein the product is measured for each amplification cycle may be particularly useful (eg Real time PCR Hid et al (1996) Genome Research 6, 98&994, Gibson et al (1996) Genome Research 6, 995-1001; Real time NASBA Oehlenschlager et al (1996 Nov 12) PNAS (USA) 93(23), 12811-6. Primers should be designed to preferentially amplify from an mRNA template rather than from the DNA, or be designed to create a product where the mRNA or DNA template origin can be distinguished by size or by probing. NASBA may be particularly useful as the process can be arranged such that only RNA is recognised as an initial substrate.

Detecting mRNA includes detecting MRNA in any context, or detecting that there are cells present which contain mRNA (for example, by in situ hybridisation, or in samples obtained from lysed cells). It is useful to detect the presence of MRNA or that certain cells are present (either generally or in a specific location) which can be detected by virtue of their expression of plu-1 mRNA. As noted, the presence versus absence of plu-1 mRNA may be a useful marker, or low levels versus high levels of plu-1 mRNA may be a useful marker, or specific quantified levels may be associated with a specific disease state. It will be appreciated that similar possibilities exist in relation to using the plu-1 polypeptide as a marker.

Since it is believed that plu-1 expression is derepressed in the carcinogenic state it is desirable to assess the state of activation (or derepression) of the plu-1 gene. Suitably, the methylation status of the plu-1 gene is assessed and in this case nucleic acids probes which hybridise to the plu-1 gene are useful in the practice of the invention. Changes in the methylation status of the plu-1 gene in a sample, compared to the methylation status in a normal (non-tumourigenic) sample may be indicative of carcinogenesis.

Runs of CpG dinucleotides are found clustered in regions of 1-2 kb called CpG islands, which are located in the promoter regions near the 5′ ends of many genes. Methylation of cytosine to 5-methylcytosine in these dinucleotides is a form of expression regulation sometimes referred to as ‘silencing’ or ‘transcriptional inactivation’. Hypermethylation at these sites results in gene silencing and loss of expression, whereas hypomethylation is permissive for gene expression.

In the case of Plu-1, it is suggested that in normal tissues the gene may be silenced as a result of hypermethylation, whereas in cancer cells this hypermethylation has been reversed allowing expression of the gene to occur in response to the activity of various transcription factors. As an alternative to detecting actual expression of plu-1 in cells, it may be useful to determine the methylation status of the regulatory regions of plu-1. The following are some of the methods for determining the methylation status of a gene.

Genomic DNA is digested with methylation sensitive and insensitive restriction enzymes which cut in the CpG islands. The digested DNA is then used for a Southern blot, which is probed with a probe derived form the first exon or at least the 5′ coding region. The methylation status of the gene is deduced from the pattern of bands obtained. Suitable methylation sensitive enzymes include Eag1 and HpaII (Herman et al (1997) Cancer Research 57, 837-841).

Methylation specific PCR (MSP). Genomic DNA is treated with sodium bisulfite resulting in conversion of 5-methylcytosines into uracil residues. PCR primer sets which are specific to the original sequence (containing C residues), or specific to modified sequence (containing uracil residues) are used to perform PCR reactions. The methylation status of the original sample is deduced from the formation of the relevant PCR products (Herman et al (1996) Proc. Natl. Acad. Sci. USA 93, 9821-9826).

Sequencing of sodium bisulfite treated DNA. Genomic DNA is treated with sodium bisulfite as above, then amplified and sequenced using suitable primers (Myohanen et al (1994) DNA Sequence 5, 1-8).

Genomic DNA samples are cleaved by methylation sensitive restriction enzyme which cleaves in the CpG island, eg HpaIl. A methylation insensitive enzyme, eg MspI, may be used as a control. HpaII and MspI both recognise and cleave at CCGG sites. The digested DNA is then used as the substrate for a PCR reaction using primers flanking the restriction site. When HpaII is used a PCR product is only formed when methylation is present (Lee et al (1997) Cancer Epidemiology, Biomarkers and Prevention 6, 443450).

A further aspect of the invention provides a method for determining the susceptibility of a patient to cancer comprising the steps of (i) obtaining a sample containing protein derived from the patient; and (ii) determining the relative amount, or intracellular location, of the plu-1 polypeptide.

A still further aspect of the invention provides a method of diagnosing cancer in a patient comprising the steps of (i) obtaining a sample containing protein derived from the patient; and (ii) determining the relative amount, or intracellular location, of the plu-1 polypeptide.

A yet still further aspect of the invention provides a method of predicting the relative prospects of a particular outcome of a cancer in a patient comprising the steps of (i) obtaining a sample containing protein derived from the patient; and (ii) determining the relative amount, or intracellular location, of the plu-1 polypeptide.

An increased level of plu-1 in a sample compared with a known normal (non-tumourigenic) tissue sample is suggestive of a tumorigenic sample. Typically, the level in a tumorigenic sample is at least 2-fold, preferably at least 5-fold and more preferably or at least 10-fold more in a tumorigenic sample compared to a known normal tissue sample. It may also be useful to measure the presence (tumour) versus absence (normal) of plu-1 polypeptide in some circumstances, such as when assessing breast tissue.

It will be appreciated that detecting the presence of an increased level of plu-1 in a cell compared to the level present in a normal cell may suggest that the patient will benefit from a particular form of treatment, such as treatment with a plu-1 tumour vaccine as herein disclosed.

The methods of the invention also include the measurement and detection of the plu-1 polypeptide in test samples and their comparison in a control sample.

The sample containing protein derived from the patient is conveniently a sample of the tissue in which cancer is suspected or in which cancer may be or has been found. These methods may be used for any cancer, but they are particularly suitable in respect of cancer of the breast or ovary, the methods are especially suitable in respect of cancer of the breast. Methods of obtaining suitable samples are described in relation to earlier methods. The sample may also be blood, serum or lymph nodes which may be particularly useful in determining whether a cancer has spread.

The methods of the invention involving detection of the plu-1 polypeptide are particularly useful in relation to historical samples such as those containing paraffin-embedded sections of tumour samples.

The relative amount of the plu-1 polypeptide may be determined in any suitable way.

It is preferred if the relative amount of the plu-1 polypeptide is determined using a molecule which selectively binds to plu-1 polypeptide Suitably, the molecule which selectively binds to plu-1 is an antibody. The antibody may also bind to a natural variant or fragment of plu-1 polypeptide.

Antibodies which selectively bind plu-1 polypeptide but which do not substantially bind any other polypeptide such as RBP-1 or RBP-2 are described above.

The antibodies for use in the methods of the in invention may be monoclonal or polyclonal.

By “the relative amount of plu-1 polypeptide” is meant the amount of plu-1 polypeptide per unit mass of sample tissue or per unit number of sample cells compared to the amount of plu-1 polypeptide per unit mass of known normal tissue or per unit number of normal cells. The relative amount may be determined using any suitable protein quantitation method. In particular, it is preferred if antibodies are used and that the amount of plu-1 is determined using methods which include quantititative western blotting, enzyme-linked immunosorbent assays (ELISA) or quantitative immunohistochemistry.

As noted above, an increased level of plu-1 in a sample compared with a known normal tissue sample is suggestive of a tumorigenic sample. In relation to breast tissue, the presence of plu-1, compared to its absence, is suggestive of carcinogenesis.

In a preferred embodiment of the invention, antibodies will immunoprecipitate plu-1 proteins from solution as well as react with plu-1 protein on western or imnunoblots of polyacrylamide gels. In another preferred embodiment, antibodies will-detect plu-1 proteins in paraffin or frozen tissue sections, using immunocytochemical techniques.

Preferred embodiments relating to methods for detecting plu-1 include enzyme linked immunosorbent assays (ELISA), radioimmunoassay (RIA), immunoradiometric assays (IRMA) and immunoenzymatic assays (IEMA), including sandwich assays using monoclonal and/or polyclonal antibodies. Exemplary sandwich assays are described by David et al in U.S. Pat. Nos. 4,376,110 and 4,486,530, hereby incorporated by reference. Methods for detection also include immuno-fluoresence. Automated and semi-automated image analysis systems may be of use.

Several formats for quantitative immunoassays are known. Such systems may incorporate: more than one antibody which binds the antigen; labelled or unlabelled antigen (in addition to any contained in the sample); and a variety of detection systems including radioisotope, colourimetric, fluorimetric, chemiluminescent, and enhanced chemiluminescent; enzyme catalysis may or may not be involved. Immunoassays may be homogenous systems, where no separation of bound and unbound reagents takes place, or heterogeneous systems involving a separation step.

Such assays are commonly referred to as eg enzyme-linked luminescent immunoassays (ELLIA), fluorescence enzyme immunoassay (FEIA), fluorescence immunoassay (FIA), enzyme immunoassay (EIA), luminescent immunoassay (LIA), latex photometrix immunoassay (LPIA).

In a further embodiment, the intracellular location of plu-1 is measured. If the intracellular location in a tissue sample is significantly different from that in a normal (non-tumorigenic) tissue sample, this may be indicative of a cancerous change in the sample.

A further aspect of the invention provides the use of a molecule which selectively binds to plu-1 polypeptide or a natural fragment or variant thereof in a method of diagnosing cancer; or in the manufacture of a reagent for diagnosing cancer.

The following therapeutic methods are particularly suited to, although not limited to, female patients.

A further aspect of the invention provides a method of treating cancer, the method comprising administering to the patient an effective amount of plu-1 polypeptide or a fragment or variant or fusion thereof, or an effective amount of a nucleic acid encoding plu-1 polypeptide or a fragment or variant or fusion thereof, wherein the amount of said polypeptide or amount of said nucleic acid is effective to provoke an anticancer cell immune response in said patient.

The peptide or peptide-encoding nucleic acid constitutes a tumour or cancer vaccine. It may be administered directly into the patient, into the affected organ or systemically, or applied ex vivo to cells derived from the patient or a human cell line which are subsequently administered to the patient, or used in vitro to select a subpopulation from immune cells derived from the patient, which are then re-administered to the patient. If the nucleic acid is administered to cells in vitro, it may be useful for the cells to be transfected so as to co-express immune-stimulating cytokines, such as interleukin-2. The plu-1 polypeptide or peptide fragment may be substantially pure, or combined with an immune-stimulating adjuvant such as Detox, or used in combination with immune-stimulatory cytokines, or be administered with a suitable delivery system, for example liposomes. The plu-1 polypeptide or peptide fragment may also be conjugated to a suitable cancer such as keyhole limpet haemocyanin (KLH) or mannan (see WO 95/18145 and Longenecker et al (1993) Ann. NY Acad. Sci. 690, 276-291). The peptide may also be tagged, or be a fusion protein. The nucleic acid may be substantially pure, or contained in a suitable vector or delivery system Suitable vectors and delivery systems include viral, such as systems based on adenovirus, vaccinia virus, retroviruses, herpes virus, adeno-associated virus or hybrids containing elements of more than one virus. Non-viral delivery systems include cationic lipids and cationic polymers as are well known in the art of DNA delivery. Physical delivery, such as via a “gene-gun” may also be used. The peptide or peptide encoded by the nucleic acid may be a fusion protein, for example with β2-microglobulin.

The peptide fragment for use in a cancer vaccine may be any suitable length fragment of the plu-1 polypeptide. In particular, it may be a suitable 9-mer peptide or a suitable 7-mer or 8-mer peptide. Longer peptides may also be suitable, but 9-mer peptides are preferred. Multiple epitopes, derived from the plu-1 polypeptide, may also be used. As noted previously, the term peptide includes a peptidomimetic. It also includes glycopeptides.

Suitably, any nucleic acid administered to the patient is sterile and pyrogen free. Naked DNA may be given intramuscularly or intradermally or subcutaneously. The peptides may be given intramuscularly, intradermally or subcutaneously.

It is particularly useful if the cancer vaccine is administered in a manner which produces a cellular immune response, resulting in cytoxic tumour cell killing by NK cells or cytotoxic T cells (CTLs). Strategies of administration which activate T helper cells are particularly useful. It may also be useful to stimulate a humoral response. It may be useful to co-adminster certain cytokines to promote such a response, for example interleukin-2, interleukin-12, interleukin-6, or interleukin-10. In addition, it may be useful to combine vaccination with strategies which increase MHC presentation on the surface of tumour cells, for example by co-administration of interferon-gamma or retinoic as is described in Nouri et al (1992) Eur. J. Cancer 28A, 1110-1115 and Seliger et al (1997) Scand. J. Immunol. 46, 625-632. It may also be desirable to make modifications to the antigen (plu-1 polypeptide or part thereof) to enhance its presentation to the immune system, for example which directs plu-1 presentation via the Class II pathway.

It may also be useful to target the vaccine to specific cell populations, for example antigen presenting cells, either by the site of injection, use of targeting vectors and delivery systems, or selective purification of such a cell population from the patient and ex vivo administration of the peptide or nucleic acid (for example dendritic cells may be sorted as described in Zhou et al (1995) Blood 86, 3295-3301; Roth et al (1996) Scand. J. Immunology 43, 646-651). For example, targeting vectors may comprise a tissue- or tumour-specific promoter which directs expression of the antigen at a suitable place.

Patients to whom the therapy is to be given, may have their tumours typed for overexpression or abnormal expression of plu-1, or particularly in relation to breast tissue, expression of plu-1. Expression of plu-1-is substantially absent from normal breast tissue.

A further aspect of the invention therefore provides a vaccine effective against cancer or cancer or tumour cells comprising an effective amount of plu-1 polypeptide or a fragment or variant thereof, or comprising a nucleic acid encoding plu-1 polypeptide or a fragment or variant thereof.

It is particularly preferred if the vaccine is a nucleic acid vaccine. It is known that inoculation with a nucleic acid vaccine, such as a DNA vaccine, encoding a polypeptide leads to a T cell response. In particular, MHC class I and class II-mediated interactions can be elicited.

Peptide products derived by cytosolic degradation of fragments of tumour-specific proteins, expressed de novo, are believed to gain access to the presentational pathways, mimicking the presentation of, for example, viral proteins, in infected cells. Presentation as neo-antigens or surrogate antigens in this novel context is believed to be a means of breaking immunological tolerance, and may lead to the generation of a tumour-specific immune response.

Conveniently, the nucleic acid vaccine may comprise any suitable nucleic acid delivery means. The nucleic acid, preferably DNA, may be naked (ie with substantially no other components to be administered) or it may be delivered in a liposome or as part of a viral vector delivery system.

It is believed that uptake of the nucleic acid and expression of the encoded polypeptide by dendritic cells may be the mechanism of priming of the immune response.

It is preferred if the vaccine, such as DNA vaccine, is administered into the muscle. It is also preferred if the vaccine is administered onto the skin.

It is preferred if the nucleic acid vaccine is administered with an adjuvant such as BCG or alum. Other suitable adjuvants include Aquila's QS21 stimulon (Aquila Biotech, Worcester, Mass., USA) which is derived from saponin, mycobacterial extracts and synthetic bacterial cell wall mimics, and proprietory adjuvants such as Ribi's Detox. Quil A, another saponin-derived adjuvant, may also be used (Superfos, Denmark).

Other adjuvants such as Freund's may also be useful. It may also be useful to give the plu-1 antigen conjugated to keyhole limpet haemocyanin, preferably also with an adjuvant.

Polynucleotide-mediated immunization therapy of cancer is described in Conry et al (1996) Seminars in Oncology 23, 135-147; Condon et al (1996) Nature Medicine 2, 1122-1127; Gong et al (1997) Nature Medicine 3, 558-561; Zhai et al (1996) J. Immunol. 156, 700-710; Graham et al (1996) Int J. Cancer 65, 664-670; and Burchell et al (1996) pp 309-313 In: Breast Cancer, Advances in biology and therapeutics, Calvo et al (eds), John Libbey Eurotext, all of which are incorporated herein by reference.

The plu-1 polypeptide is an appropriate target for a cell-mediated response to cancer or tumour cells which express the plu-1 polypeptide.

Therapeutic response to a cancer vaccine may usefully be monitored. Suitably, plu-1 specific antibody and CTL responses are monitored using methods well known in the art to assess the efficacy of the therapeutic response. Lymphoblastic transformation assays, lymphokine release assays, CTL response assays and serologic assays may be used as disclosed in Example 4.

Detection of antigen-specific T lymphocytes by fluorescent-activated cell sorting (FACS) may also be used and is described in Altman et al (1996) Science 274, 94-96 and in WO 96/26962.

A further aspect of the invention provides a method for producing activated cytotoxic T lymphocytes (CTL) in vitro, the method comprising contacting in vitro CTL with antigen-loaded human class I MHC molecules expressed on the surface of a suitable cell for a period of time sufficient to activate, in an antigen specific manner, said CTL wherein the antigen is an antigenic peptide derived from the plu-1 polypeptide.

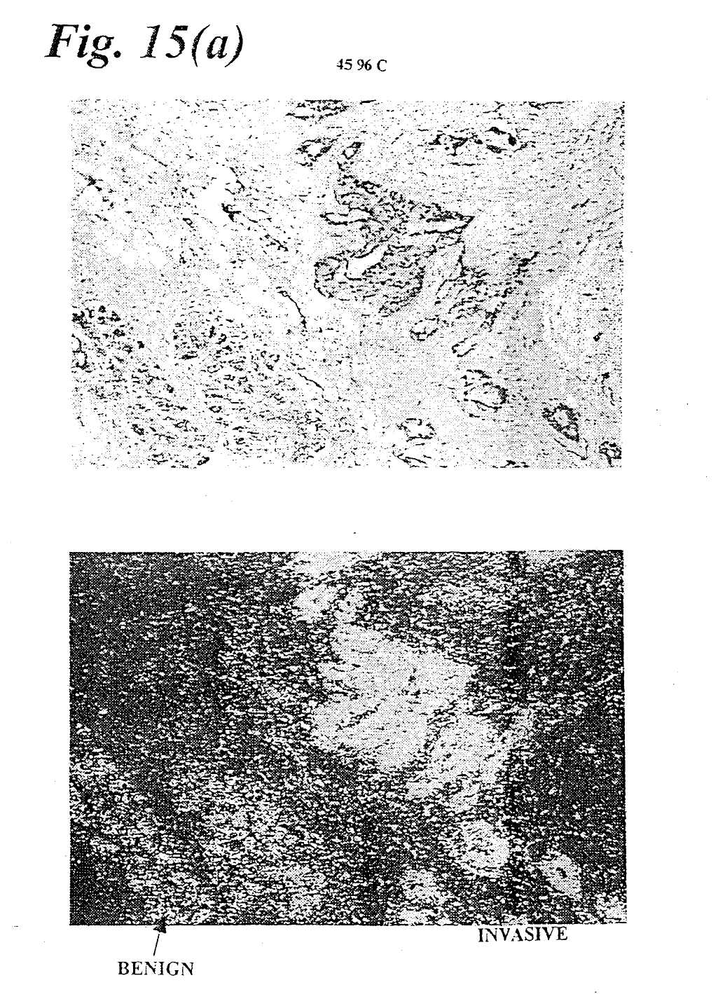

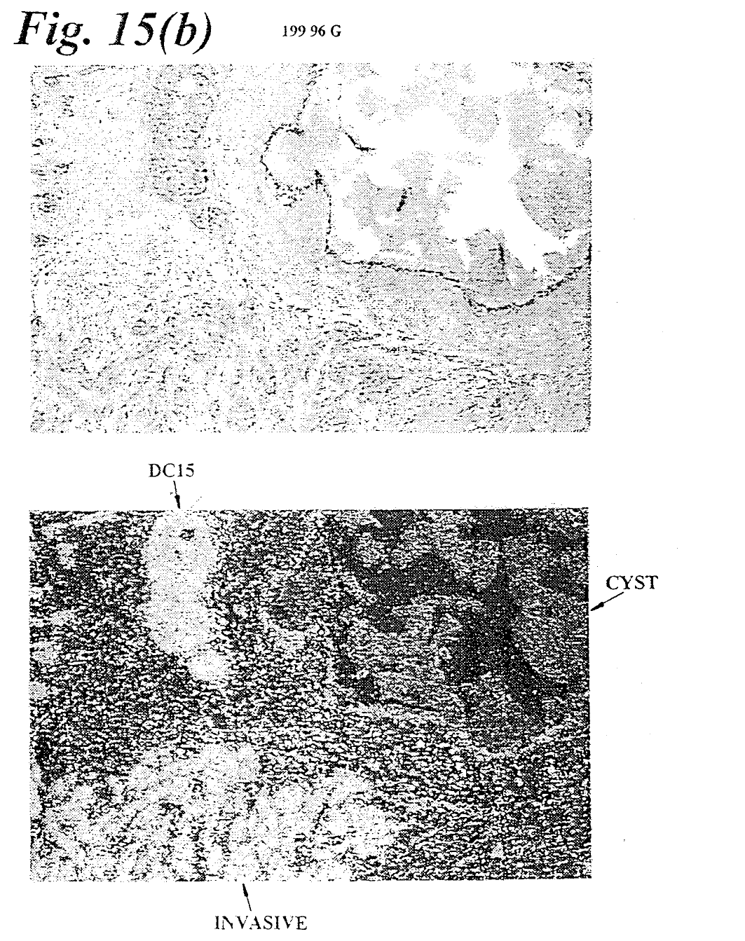

Suitably, the CTL are CD8⁺ cells but they may be CD4⁺ cells. The MHC class I molecules may be expressed on the surface of any suitable cell and it is preferred if the cell is one which does not naturally express MHC class I molecules (in which case the cell is transfected to express such a molecule) or, if it does, it is defective in the antigen-processing or antigen-presenting pathways. In this way, it is possible for the cell expressing the MHC class I molecule to be primed substantially completely with a chosen peptide antigen before activating the CTL. The antigen is any antigenic peptide derived from the plu-1 polypeptide. Peptides which are believed to bind to MHC class I molecules are shown in FIG. 15; however, any suitable peptides derived from plu-1 may be used. It is preferred if the peptides are nonapeptides; it is further preferred if the nonapeptides are specific for plu-1 and are peptides which are not found in any of RBP-1, RBP-2 or any other polypeptide.

The antigen-presenting cell (or stimulator cell) typically has an MHC class I molecule on its surface and preferably is substantially incapable of itself loading said MHC class I molecule with the selected antigen. As is described in more detail below, the MHC class I molecule may readily be loaded with the selected antigen in vitro.

Conveniently, said antigen-presenting cell is a mammalian cell defective in the expression of a peptide transporter such that, when at least part of said selected molecule is a peptide, it is not loaded into said MHC class I molecule.

Preferably the mammalian cell lacks or has a reduced level or has reduced function of the TAP peptide transporter. Suitable cells which lack the TAP peptide transporter include T2, RMA-S and Drosophila cells. TAP is the Transporter Associated with antigen Processing.

Thus, conveniently the cell is an insect cell such as a Drosophila cell.

The human peptide loading deficient cell line T2 is available from the American Type Culture Collection, 12301 Parklawn Drive, Rockville, Md. 20852, USA under Catalogue No CRL 1992; the Drosophila cell line Schneider line 2 is available from the ATCC under Catalogue No CRL 19863; the mouse RMA-S cell line is described in Karre and Ljunggren (1985) J. Exp. Med. 162, 1745, incorporated herein by reference.

In a preferred embodiment the stimulator cell is a host cell (such as a T2, RMA-S or Drosophila cell) transfected with a nucleic acid molecule capable of expressing said MHC class I molecule. Although T2 and RMA-S cells do express before transfection HLA class I molecules they are not loaded with a peptide.

Mammalian cells can be transfected by methods well known in the art. Drosophila cells can be transfected, as described in Jackson et al (1992) proc. Natl. Acad. Sci. USA 89, 12117, incorporated herein by reference.

Conveniently said host cell before transfection expresses substantially no MHC class I molecules.

It is also preferred if the stimulator cell expresses a molecule important for T cell costimulation such as any of B7.1, B7.2, ICAM-1 and LFA 3.

The nucleic acid sequences of numerous MHC class I molecules, and of the costimulator molecules, are publicly available from the GenBank and EMBL databases.

It is particularly preferred if substantially all said MHC class I molecules expressed in the surface of said stimulator cell are of the same type.

The term HLA may be used interchangeably with MHC in relation to human class I molecules.

HLA class I in humans, and equivalent systems in other animals, are genetically very complex. For example, there are at least 110 alleles of the HLA-B locus and at least 90 alleles of the HLA-A locus. Although any HLA class I (or equivalent) molecule is useful in this aspect of the invention, it is preferred if the stimulator cell presents at least part of the selected molecule in an HLA class I molecule which occurs at a reasonably high frequency in the human population. It is well known that the frequency of HLA class I alleles varies between different ethnic groupings such as Caucasian, African, Chinese and so on. At least as far as the Caucasian population is concerned it is preferred that HLA class I molecule is encoded by an HLA-A2 allele, or an HLA-A1 allele or an HLA-A3 allele or an HLA-B27 allele. HLA-A2 is particularly preferred.

In a further embodiment, combinations of HLA molecules may also be used. For example, a combination of HLA-A2 and HLA-A3 covers 74% of the Caucasian population.

In a still further embodiment, multiple epitopes, such as multiple plu-1 epitopes, or combinations of plu-1 epitopes with epitopes from other tumour antigens such as MUC-1 or CEA may be used. The use of recombinant polyepitope vaccines for the delivery of multiple CD8 CTL epitopes is described in Thomson et al (1996) J. Immunol. 157, 822-826 and WO 96/03144, both of which are incorporated herein by reference.

It will be appreciated that although Class I epitopes may be used in a vaccine, it is also desirable to use Class II epitopes derived from the plu-1 polypeptide. Examples of methods for predicting Class II binding peptides are disclosed in Hammer et al (1994) J. Exp. Med. 180, 2353-2358 and Roberts et al (1996) AIDS Res. Hum. Retroviruses 12, 593-610.

A convenient method of activating CTL (CD8⁺ cells) is described in WO 93/17095, incorporated herein by reference.

A number of other methods may be used for generating CTL in vitro. For example, the methods described in Peoples et al (1995) Proc. Natl. Acad. Sci. USA 92, 432436 and Kawakami et al (1992) J. Immunol. 148, 638-643 use autologous tumour-infiltrating lymphocytes in the generation of CTL. Plebanski et al (1995) Eur. J. Immunol. 25, 1783-1787 makes use of autologous peripheral blood lymphocytes (PLBs) in the preparation of CTL. Jochmus et al (1997) J. Gen. Virol. 78, 1689-1695 describes the production of autologous CTL by employing pulsing dendritic cells with peptide or polypeptide, or via infection with recombinant virus.

Hill et at (1995) J. Exp. Med. 181, 2221-2228 and Jerome et al (1993) J. Immunol. 151, 1654-1662 make use of B cells in the production of autologous CTL. In addition, macrophages pulsed with peptide or polypeptide, or infected with recombinant virus, may be used in the preparation of autologous CTL.

Allogeneic cells may also be used in the preparation of CTL. For example, in addition to Drosophila cells and T2 cells, other cells may be used to present antigens such as CHO cells, baculovirus-infected insects cells, bacteria, yeast, vaccinia-infected target cells. In addition plant viruses may be used (see, for example, Porta et al (1994) Virology 202, 449-955 which describes the development of cowpea mosaic virus as a high-yielding system for the presentation of foreign peptides.

MHC Class II responses may be induced by linkage of plu-1 peptides to carriers such as keyhole limpet haemocyanin and tetanus toxin, which induces a T helper response, or by linkage to lysosomal-associated membrane protein (LAMP-1) to direct the antigen into the MHC Class II pathway (see, for example, Wu et al (1995) Proc. Natl. Acad. Sci. USA 92, 11671-11675).

Exogenously applied plu-1 peptides may be linked to a HIV tat peptide to direct them into the MHC Class I pathway for presentation by CTL (see, for example, Kim et al (1997) J. Immunol 159, 166&1668.

The activated CTL which are directed against plu-1 polypeptide are useful in therapy.

A further aspect of the invention provides a method of specifically killing target cells in a human patient which target cells express the plu-1 polypeptide, the method comprising (1) obtaining a sample containing precursor CTL from said patient, (2) contacting, in vitro, said CTL with antigen-loaded human class I MHC molecules expressed on the surface of a suitable cell for a period of time sufficient to activate, in an antigen specific manner, said CTL wherein the antigen is an antigenic peptide derived from the plu-1 polypeptide. Preferably, the human patient is a patient with a cancer that expresses the plu-1 polypeptide. Most preferably the patient to be treated is one with breast cancer or ovarian cancer.

A still further aspect of the invention provides a method of treating a patient with cancer, the method comprising obtaining dendritic cells from said patient, contacting said dendritic cells with an antigenic peptide derived from the plu-1 polypeptide, or with a polynucleotide encoding said antigenic peptide, ex vivo, and reintroducing the so treated dendritic cells into the patient.

Suitably, the dendritic cells are autologous dendritic cells which are pulsed with an antigenic peptide derived from the plu-1 polypeptide. The antigenic peptide may be any suitable antigenic peptide which gives rise to an appropriate T cell response. T-cell therapy using autologous dendritic cells pulsed with peptides from a tumour associated antigen is disclosed in Murphy et al (1996) The Prostate 29, 371-380 and Tjua et al (1997) The Prostate 32, 272-278.

In a further embodiment the dendritic cells are contacted with a polynucleotide which encodes an antigenic peptide derived from plu-1. The polynucleotide may be any suitable polynucleotide and it is preferred that it is capable of transducing the dendritic cell thus resulting in the presentation of plu-1 peptides and induction of immunity. It will be appreciated that the “antigenic peptide” may be complete plu-1 or any suitable fragment thereof.

Conveniently, the polynucleotide may be comprised in a viral polynucleotide or virus. For example, adenovirus-transduced dendritic cells have been shown to induce antigen-specific antitumour immunity in relation to MUC1 (see Gong et al (1997) Gene Ther. 4, 1023-1028). Similarly, adenovirus-based systems may be used (see, for example, Wan et al (1997) Hum. Gene Ther. 3, 1355-1363); retroviral systems may be used (Specht et al (1997) J. Exp. Med. 186, 1213-1221 and Szabolcs et al (1997) Blood 90, 2160-2167); particle-mediated transfer to dendritic cells may also be used (Tuting et al (1997) Eur. J. Immunol. 27, 2702-2707); and RNA may also be used (Ashley et al (1997) J. Exp. Med. 186, 1177-1182).

A further aspect of the invention provides a method of treating a patient with cancer the method comprising administering to the patient an effective amount of a plu-1 antisense agent.

By “plu-1 antisense agent” is included agents which bind to plu-1 mRNA and, preferably, inhibit its translation; also included are agents which bind to the plu-1 gene and inhibit its transcription. Antisense agents can be designed by reference to the plu-1 sequences disclosed herein. Preferably, the antisense agent is an oligonucleotide.

Oligonucleotides are subject to being degraded or inactivated by cellular endogenous nucleases. To counter this problem, it is possible to use modified oligonucleotides, eg having altered internucleotide linkages, in which the naturally occurring phosphodiester linkages have been replaced with another linkage. For example, Agrawal et al (1988) Proc. Natl. Acad. Sci. USA 85, 7079-7083 showed increased inhibition in tissue culture of HIV-1 using oligonucleotide phosphoramidates and phosphorothioates. Sarin et al (1988) Proc. Natl. Acad. Sci. USA 85, 7448-7451 demonstrated increased inhibition of HIV-1 using oligonucleotide methylphosphonates. Agrawal et al (1989) Proc. Natl. Acad. Sci. USA 86, 7790-7794 showed inhibition of HIV-1 replication in both early-infected and chronically infected cell cultures, using nucleotide sequence-specific oligonucleotide phosphorothioates. Leither et al (1990) Proc. Natl. Acad. Sci USA 87, 3430-3434 report inhibition in tissue culture of influenza virus replication by oligonucleotide phosphorothioates.

Oligonucleotides having artificial linkages have been shown to be resistant to degradation in vivo. For example, Shaw et al (1991) in Nucleic Acids Res. 19, 747-750, report that otherwise unmodified oligonucleotides become more resistant to nucleases in vivo when they are blocked at the 3′ end by certain capping structures and that uncapped oligonucleotide phosphorothioates are not degraded in vivo.

A detailed description of the H-phosphonate approach to synthesizing oligonucleoside phosphorothioates is provided in Agrawal and Tang (1990) Tetrahedron Letters 31, 7541-7544, the teachings of which are hereby incorporated herein by reference. Syntheses of oligonucleoside methylphosphonates, phosphorodithioates, phosphoramidates, phosphate esters, bridged phosphoramidates and bridge phosphorothioates are known in the art. See, for example, Agrawal and Goodchild (1987) Tetrahedon Letters 28, 3539; Nielsen et al (1988) Tetrahedron Letters 29, 2911; Jager et al (1988) Biochemistry 27, 7237; Uznanski et al (1987) Tetrahedron Letters 25, 3401; Bannwarth (1988) Helv. Chim. Acta. 71, 1517; Crosstick and Vyle (1989) Tetrahedron Letters 30, 4693; Agrawal et al (1990) Proc. Natl. Acad. Sci. USA 87, 1401-1405, the teachings of which are incorporated herein by reference. Other methods for synthesis or production also are possible. In a preferred embodiment the oligonucleotide is a deoxyribonucleic acid (DNA), although ribonucleic acid (NA) sequences may also be synthesized and applied.

The oligonucleotides useful in the invention preferably are designed to resist degradation by endogenous nucleolytic enzymes. In vivo degradation of oligonucleotides produces oligonucleotide breakdown products of reduced length. Such breakdown products are more likely to engage in non-specific hybridization and are less likely to be effective, relative to their full-length counterparts. Thus, it is desirable to use oligonucleotides that are resistant to degradation in the body and which are able to reach the targeted cells. The present oligonucleotides can be rendered more resistant to degradation in vivo by substituting one or more internal artificial internucleotide linkages for the native phosphodiester linkages, for example, by replacing phosphate with sulphur in the linkage. Examples of linkages that may be used include phosphorothioates, methylphosphonates, sulphone, sulphate, ketyl, phosphorodithioates, various phosphoramidates, phosphate esters, bridged phosphorothioates and bridged phosphoramidates. Such examples are illustrative, rather than limiting, since other internucleotide linkages are known in the art. See, for example, Cohen, (1990) Trends in Biotechnology. The synthesis of oligonucleotides having one or more of these linkages substituted for the phosphodiester internucleotide linkages is well known in the art, including synthetic pathways for producing oligonucleotides having mixed internucleotide linkages.

Oligonucleotides can be made resistant to extension by endogenous enzymes by “capping” or incorporating similar groups on the 5′ or 3′ terminal nucleotides. A reagent for capping is commercially available as Amino-Link II™ from Applied BioSystems Inc, Foster City, Calif. Methods for capping are described, for example, by Shaw et al (1991) Nucleic Acids Res. 19, 747-750 and Agrawal et al (1991) Proc. Natl. Acad. Sci. USA 88(17), 7595-7599, the teachings of which are hereby incorporated herein by reference.

A further method of making oligonucleotides resistant to nuclease attack is for them to be “self-stabilized” as described by Tang et al (1993) Nucl. Acids Res. 21, 2729-2735 incorporated herein by reference. Self-stabilized oligonucleotides have hairpin loop structures at their 3′ ends, and show increased resistance to degradation by snake venom phosphodiesterase, DNA polymerase I and fetal bovine serum. The self-stabilized region of the oligonucleotide does not interfere in hybridization with complementary nucleic acids, and pharmacokinetic and stability studies in mice have shown increased in vivo persistence of self-stabilized oligonucleotides with respect to their linear counterparts.

In accordance with the invention, the inherent binding specificity of antisense oligonucleotides characteristic of base pairing is enhanced by limiting the availability of the antisense compound to its intend locus in vivo, permitting lower dosages to be used and minimizing systemic effects. Thus, oligonucleotides are applied locally to achieve the desired effect. The concentration of the oligonucleotides at the desired locus is much higher than if the oligonucleotides were administered systemically, and the therapeutic effect can be achieved using a significantly lower total amount. The local high concentration of oligonucleotides enhances penetration of the targeted cells and effectively blocks translation of the target nucleic acid sequences.

The oligonucleotides can be delivered to the locus by any means appropriate for localized administration of a drug. For example, a solution of the oligonucleotides can be injected directly to the site or can be delivered by infusion using an infusion pump. The oligonucleotides also can be incorporated into an implantable device which when placed at the desired site, permits the oligonucleotides to be released into the surrounding locus.

The oligonucleotides are most preferably administered via a hydrogel material The hydrogel is noninflammatory and biodegradable. Many such materials now are known, including those made from natural and synthetic polymers. In a preferred embodiment, the method exploits a hydrogel which is liquid below body temperature but gels to form a shape-retaining semisolid hydrogel at or near body temperature. Preferred hydrogel are polymers of ethylene oxide-propylene oxide repeating units. The properties of the polymer are dependent on the molecular weight of the polymer and the relative percentage of polyethylene oxide and polypropylene oxide in the polymer. Preferred hydrogels contain from about 10 to about 80% by weight ethylene oxide and from about 20 to about 90%o by weight propylene oxide. A particularly preferred hydrogel contains about 70% polyethylene oxide and 30% polypropylene oxide. Hydrogels which can be used are available, for example, from BASF Corp., Parsippany, N.J., under the tradename Pluronic^{R}.

In this embodiment, the hydrogel is cooled to a liquid state and the oligonucleotides are admixed into the liquid to a concentration of about 1 mg oligonucleotide per gram of hydrogel. The resulting mixture then is applied onto the surface to be treated, for example by spraying or painting during surgery or using a catheter or endoscopic procedures. As the polymer warms, it solidifies to form a gel, and the oligonucleotides diffuse out of the gel into the surrounding cells over a period of time defined by the exact composition of the gel.

The oligonucleotides can be administered by means of other implants that are commercially available or described in the scientific literature, including liposomes, microcapsules and implantable devices. For example, implants made of biodegradable materials such as polyanhydrides, polyorthoesters, polylactic acid and polyglycolic acid and copolymers thereof, collagen, and protein polymers, or non-biodegradable materials such as ethylenevinyl acetate (EVAc), polyvinyl acetate, ethylene vinyl alcohol, and derivatives thereof can be used to locally deliver the oligonucleotides. The oligonucleotides can be incorporated into the material as it is polymerized or solidified, using melt or solvent evaporation techniques, or mechanically mixed with the material. In one embodiment, the oligonucleotides are mixed into or applied onto coatings for implantable devices such as dextran coated silica beads, stents, or catheters.

The dose of oligonucleotides is dependent on the size of the oligonucleotides and the purpose for which is it administered. In general, the range is calculated based on the surface area of tissue to be treated. The effective dose of oligonucleotide is somewhat dependent on the length and chemical composition of the oligonucleotide but is generally in the range of about 30 to 3000 μg per square centimetre of tissue surface area.

The oligonucleotides may be administered to the patient systemically for both therapeutic and prophylactic purposes. The oligonucleotides may be administered by any effective method, for example, parenterally (eg intravenously, subcutaneously, intramuscularly) or by oral, nasal or other means which permit the oligonucleotides to access and circulate in the patient's bloodstream. Oligonucleotides administered systemically preferably are given in addition to locally administered oligonucleotides, but also have utility in the absence of local administration. A dosage in the range of from about 0.1 to about 10 grams per administration to an adult human generally will be effective for this purpose.

It will be appreciated from the foregoing that the invention contemplates the use of a polynucleotide, or antibody, to detect a cell expressing plu-1.

The invention also includes the use of plu-1 polypeptide or an active variant or fragment or derivative or fusion thereof or an active fusion of a variant or fragment or derivative thereof in an assay for identifying compounds which modulate the activity of the plu-1 polypeptide.

As noted above, the plu-1 polypeptide contains a domain which is similar to DNA binding domains from other polypeptides (see, for example, Takeuchi et al (1995) Genes Develop. 9, 1211-1222 which describes the mouse jumonji gene; Gregory et al (1996) Mol. Cell. Biol. 16, 792-799 which describes the Drosophila dead ringer gene; and Coté et al (1994) Science 265, 53-60 which describes the yeast SWI/SNF protein complex), thus, in a preferred embodiment of the assay a portion of DNA containing a plu-1 DNA binding site is immobilised on a solid support such as a filter, a multi-welled plastic plate, or a bead using methods well known in the art. Recombinant plu-1 protein, or a fragment thereof containing the DNA binding motif (amino acids 75-191) is produced using techniques methods well known in the art (described earlier in the application). The recombinant plu-1 protein is labelled using antibodies, fluorescent molecules, biotin, radioactivity or other suitable method. The labelled recombinant plu-1 protein is then applied to the immobilised DNA in the presence or absence of a test compound. The reaction is washed to remove non-specific binding activity and standard detection techniques are used to determine the relative quantity of labelled protein which remains associated with the DNA on the solid support. The degree of binding inhibition exerted by the test compound can thus be determined. The specificity of the inhibition can be determined by using the same compound in a control assay which contains an unrelated DNA binding protein and its DNA binding site.

The above assay can also be performed in reverse with unlabelled plu-1 protein immobilised on a solid support and labelled DNA added to this in the presence or absence of the test compound.

Alternatively, a plu-1 DNA binding site may be cloned upstream of a reporter gene such as luciferase and the vector introduced into a suitable host cell such as yeast or bacteria. A vector encoding plu-1 protein, or a fragment thereof is introduced into the same cell in the presence or absence of a test compound and the level of transcription of the reporter gene is monitored.

High throughput screens which make use of, for example, a scintillation proximity assay or a solid-phase, non-separation assay are described in Lerner & Saiki (1996) Anal. Biochem. 240, 185-196 and Carlsson & Haggbled (1995) Anal. Biochem. 232, 172-179. These, and other suitable, methods may be adapted for use with plu-1 polypeptide in the practice of the present invention.

Similarly, the DNA binding assay described in Gregory et al (1996) Mol. Cell Biol. 16, 792-799 may be adapted for use with plu-1 polypeptide in the practice of the present invention.

Small molecule drugs which specifically modulate (inhibit or enhance) the binding of plu-1 to its DNA binding site(s) may be useful in the treatment of cancer, particularly breast cancer.

Further aspects of the invention provide polypeptides, antibodies and nucleic acids of the invention for use in medicine.

A further aspect of the invention provides a kit of parts comprising an antibody of the invention and a control sample comprising plu-1 polypeptide or an immunoreactive fragment thereof. The kit may usefully further comprise a component for testing for a further cancer-related polypeptide such as antibodies which are reactive with one or more of the following cancer-related polypeptides, all of which are well known in the art: MAGE-1, MAGE-3, BAGE, GAGE-1, CAG-3, CEA, p53, oestrogen receptor (ER), progesterone receptor (PR), MUC1, p52 trefoil peptide, Her2, PCNA, Ki67, cyclin D, p90^{rak3}, p170 glycoprotein (mdr-1) CA-15-3, c-erbB1, cathepsin D, PSA, CA125, CA19-9, PAP, myc, cytokeratins, bcl-2, telomerase, glutathione S transferases, rad51, VEGF, thymidine phosphorylase, Flk1 or Flk2.

A still further aspect of the invention provides a kit of parts comprising a nucleic acid which hybridises selectively to plu-1 nucleic acid and a control sample comprising a plu-1 nucleic acid. The kit may usefully further comprise a nucleic acid which selectively hybridises to a further cancer-related nucleic acid such as a gene or mRNA which encodes any of the cancer-related polypeptides as described above. In addition, useful nucleic acids which may be included in the kit are those which selectively hybridise with the genes or mRNAs: ras, APC, BRCA1, BRCA2, ataxia telangiectasia (ATM), hMSH2, hMCH1, hPMS2 or hPMS1. It is preferred if the further nucleic acid is one which selectively hybridises to the gene or MRNA of any of erbB2, p53, BRCA1, BRCA2 or ATM. It is preferred if the nucleic acid does not hybridise to genes or MRNA for CA-125, CA19-9 or Ca15-3.

The kits usefully may contain controls and detection material, (for example, for immunohistochemistry, secondary antibodies labelled fluorophores, or enzymes, or biotin, or digoxygenin or the like). For immunoassays, additional components to the kit may include a second antibody to a different epitope on plu-1 (optionally labelled or attached to a support), secondary antibodies (optionally labelled or attached to a support), plu-1 polypeptide, positive and negative controls, and dilution and reaction buffers. Similar additional components may usefully be included in all of the kits of the invention.

A further aspect of the invention provides a pharmaceutical composition comprising plu-1 polypeptide or a variant or fragment or derivative or fusion thereof or a fusion of a variant or fragment or derivative thereof and a pharmaceutically acceptable carrier.

A still further aspect of the invention provides a pharmaceutical composition comprising a nucleic acid encoding plu-1 polypeptide or a variant or fragment or derivatives or fusion thereof or a fusion of a variant or fragment or derivative thereof and a pharmaceutically acceptable carrier.

The pharmaceutical compositions are sterile and pyrogen-free and conveniently they may include suitable stabilizers and preservatives.

The invention will be described in more detail with reference to the following Examples and Figures wherein

FIG. 1 shows the nucleotide sequence of a cDNA encoding the plu-1 polypeptide sequence;

FIG. 2 shows the amino acid sequence of the plu-1 polypeptide. This is a translation of the cDNA sequence given in FIG. 1 from positions 90 to 4724. Peptides used for antibody production are boxed and the DNA binding motif is underlined;

FIG. 3 shows an alignment of the plu-1 polypeptide amino acid sequence with various, known human amino acid sequences. Peptides useful for raising antisera are boxed, and peptides useful for immunotherapy are marked⇄(MHC molecules to which they may bind are indicated). None of the plu-1 homologues shown in this Figure have been shown to have tissue restricted expression: they are all ubiquitously expressed;

Rbp-2 is a cellular protein which binds to the retinoblastoma gene product (see Fattaey et al (1993) Oncogene 8, 3149-3156. Humxe169a is a human X-linked gene which is widely expressed in adult tissues and escapes X-chromosome inactivation (see Wu et al (1994) Hum. Mol. Genet. 3, 153-160). The term hssmcy means the human homologue of mouse smcy gene; the mouse smcy gene is a Y chromosome gene encoded by a region essential for spermatogenesis and expression of male-specific MHC antigens (see Agulnik et al (1994) Hum. Mol. Genet. 3, 873-878);

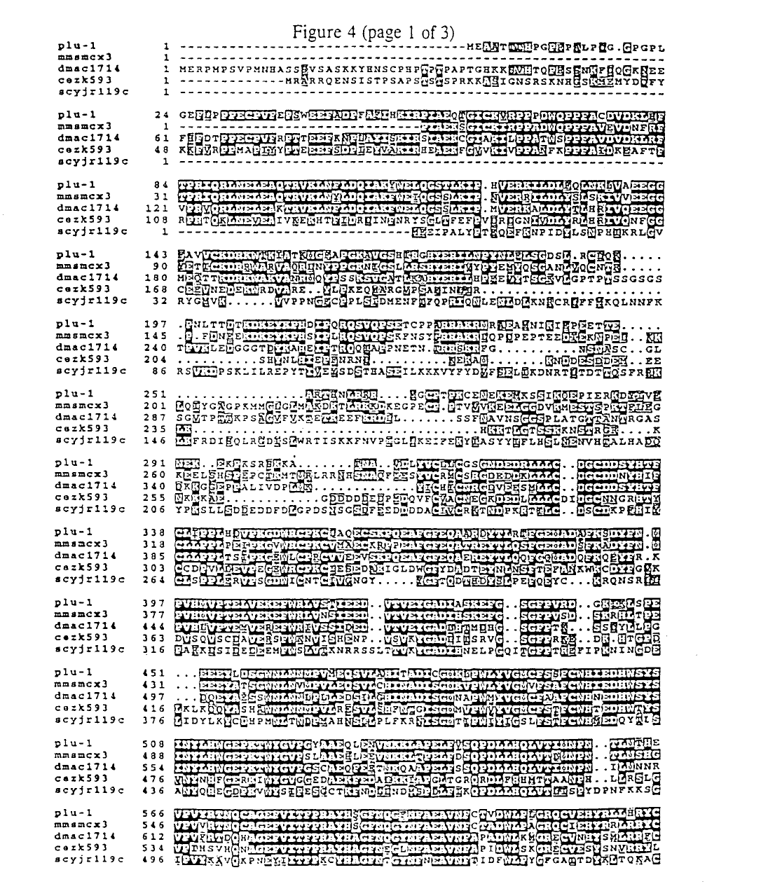

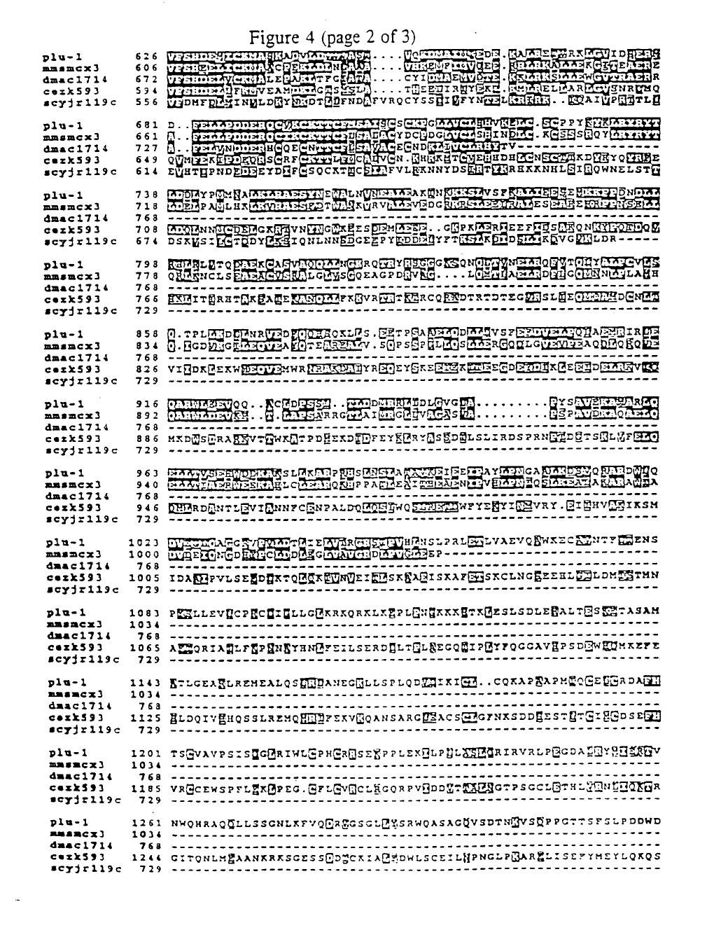

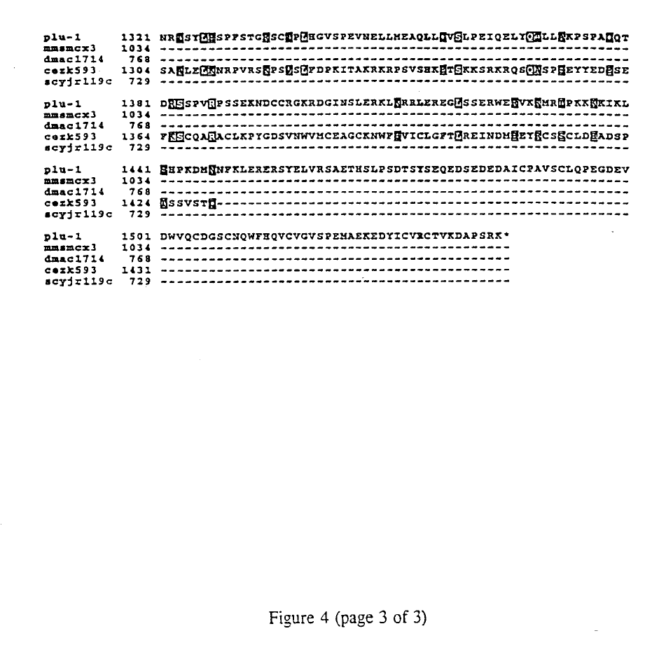

FIG. 4 shows an alignment of the plu-1 polypeptide amino acid sequence with various, known amino acid sequences from non-human species;

Mmsmcx3 is a mouse X-linked gene which escapes X-chromosome inactivation (see Agulnik et al (1994) Hum Mol. Genet. 3, 879-884);

Dmac 1714 is a Drosophila melanogaster subclone 1-a4 from P1 DSOS973 (D122) sequence (see Martin et al, GenBank Accession AC 001714);

C. elegans cosmid ZK 593 is described in Wilson et al (1994) Nature 368, 32-38.

Scyjr119c is described in Rose et al GenBank accession Z49619.

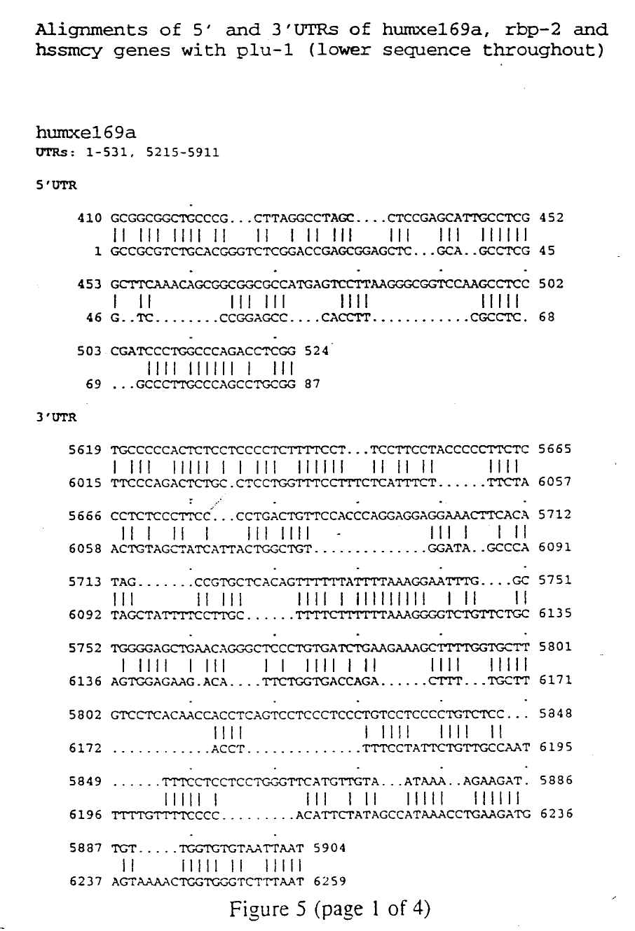

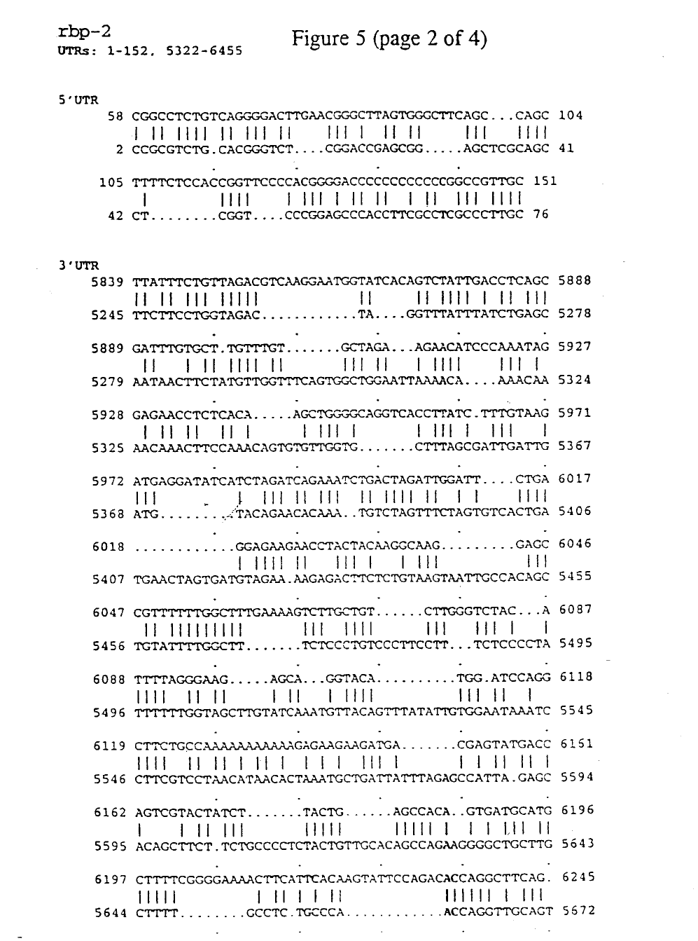

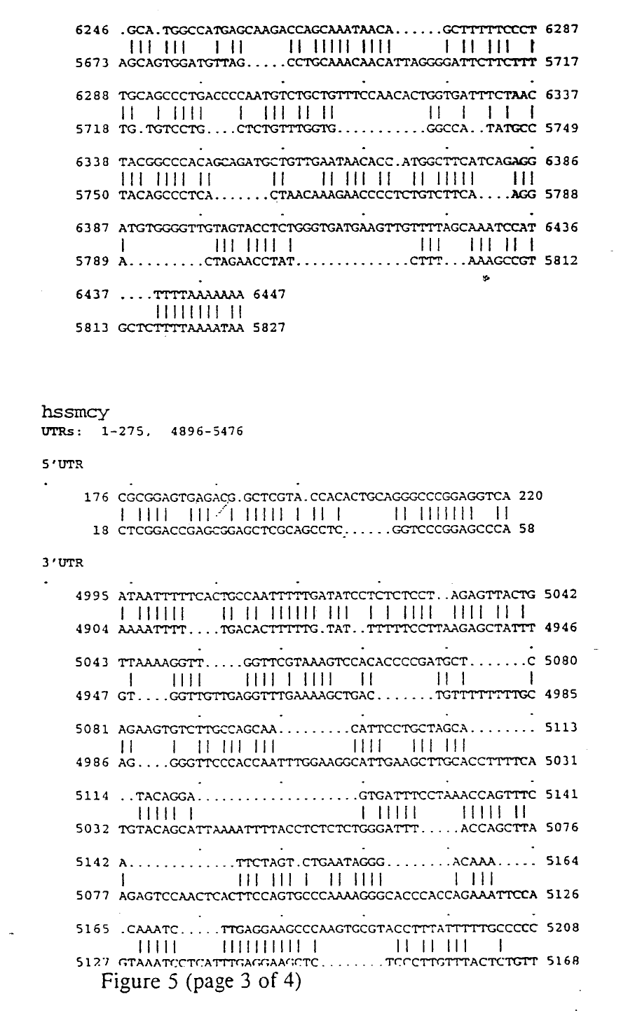

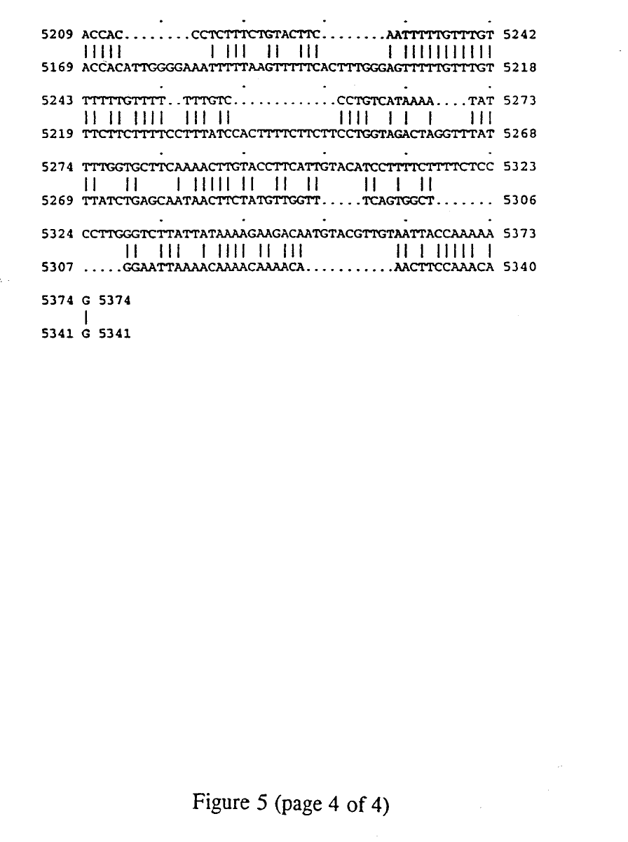

FIG. 5 shows the alignments of the 5′ and 3′ untranslated regions (UTRs) of humxe169a, rbp-2 and hssmcy genes with plu-1 (lower sequence throughout);

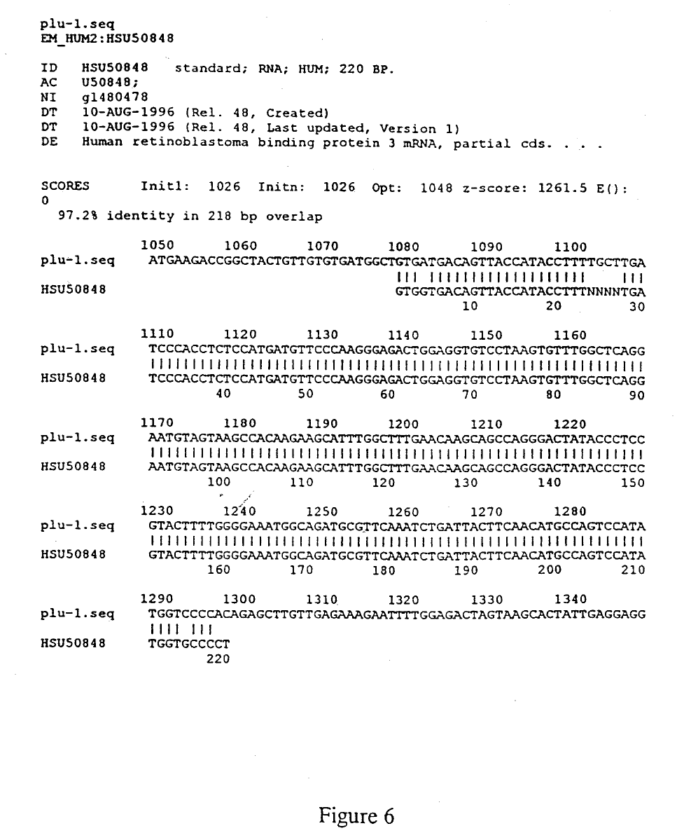

FIG. 6 shows an alignment between part of the plu-1 cDNA sequence and the sequence of HSU50848 (designated as human retinoblastoma binding protein 3);

FIG. 7 gives tables of expressed sequence tags (ESTs) which show homology to the open reading frame (ORF) plu-1 cDNA. The table on the first page gives all ESTs whereas the tables on pages 2 and 3 list the human and mouse clones, respectively;

FIG. 8 is a northern blot showing hybridisation of probes for the plu-1 gene (probe from original 253g2 clone), c-erbB2 and GAPDH with RNA from the cel cell line with and without treatment with an anti-c-erbB2 monoclonal antibody and with the MCF7 breast carcinoma cell line;

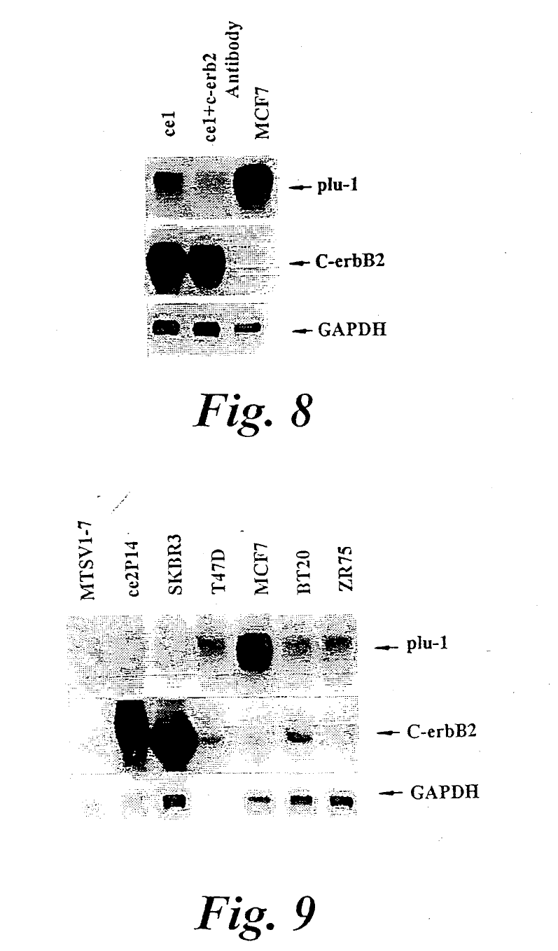

FIG. 9 is a northern blot showing hybridisation of probes for the plu-1 gene (probe from original 253g2 clone), c-erbB2 and GAPDH with RNA from the non-tumorigenic breast epithelial cell line MTSV1-7 and from various breast carcinoma cell lines;

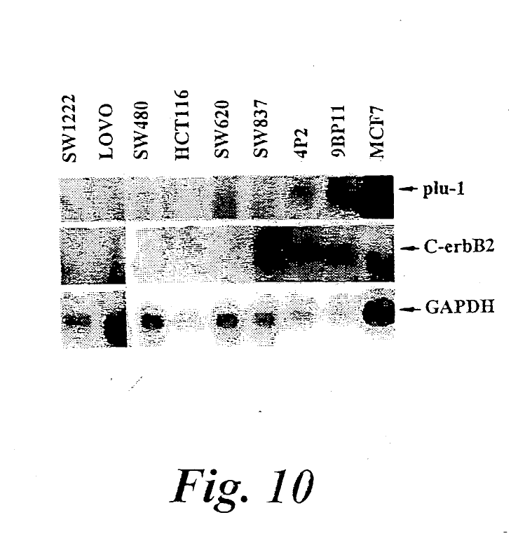

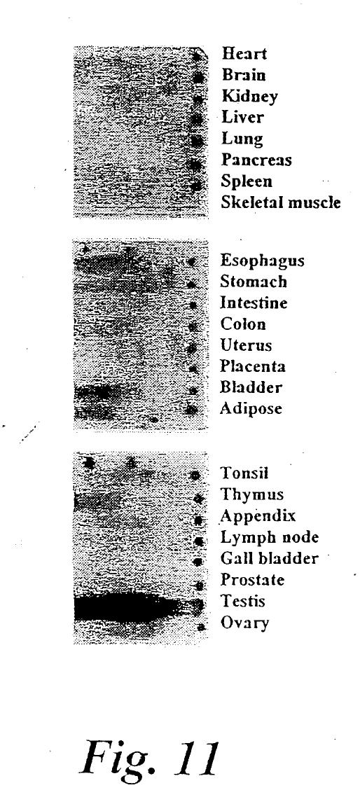

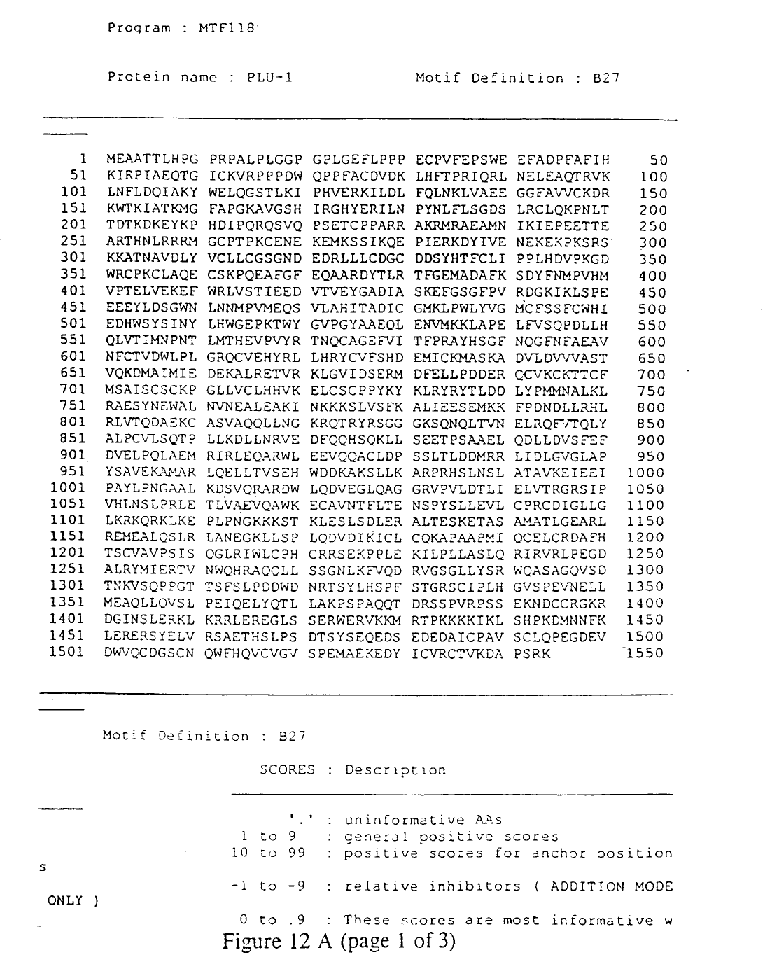

FIG. 10 is a northern blot showing hybridisation of probes for the plu-1 gene (probe from original 253g2 clone), c-erbB2 and GAPDH with RNA from colon carcinoma cell lines (SW1222, LoVo, SW480, HCT116 and SW837) and with RNA from primary cultures of breast carcinoma explants (4P2 and 9BP11). The MCF7 breast carcinoma cell line is included as control;

FIG. 11 is a multi-tissue northern blot (commercially obtained) hybridised with a probe from the plu-1 gene. Sources of RNA are as shown;

For all of the northern blots shown in FIGS. 8 to 11 a probe which contains nucleotides 3633-5559 of the plu-1 cDNA (ie mainly 3′ untranslated region was used as a probe. The probe is called 253g2.

FIG. 12 shows predicted peptides from the plu-1 polypeptide which may bind to the human class I alleles B27, A2, A3 and A11. The peptides were predicted using the MTF118 program and the HLA binding peptide predictions are ranked (scored) based on a predicted half-time of dissociation to HLA class I molecules.

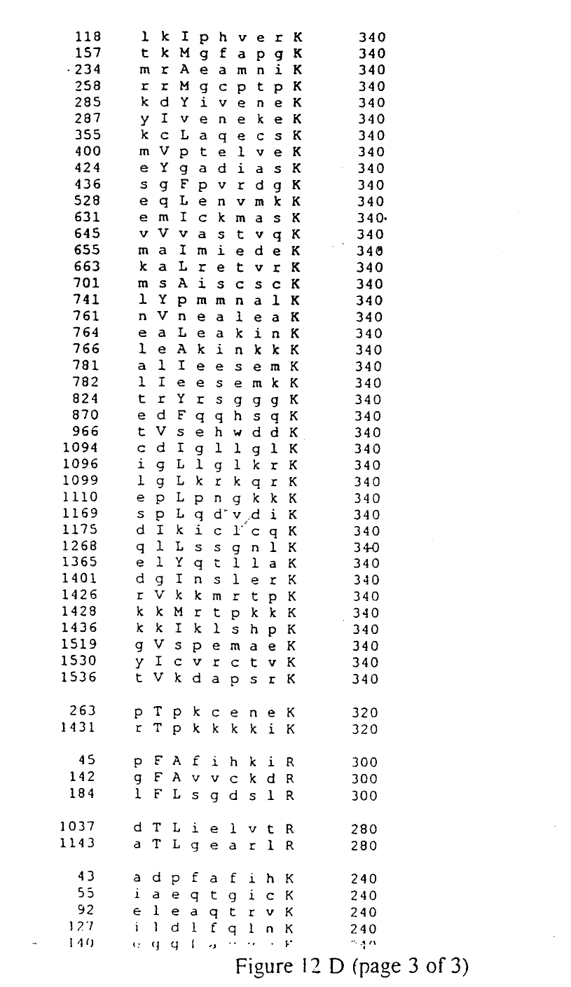

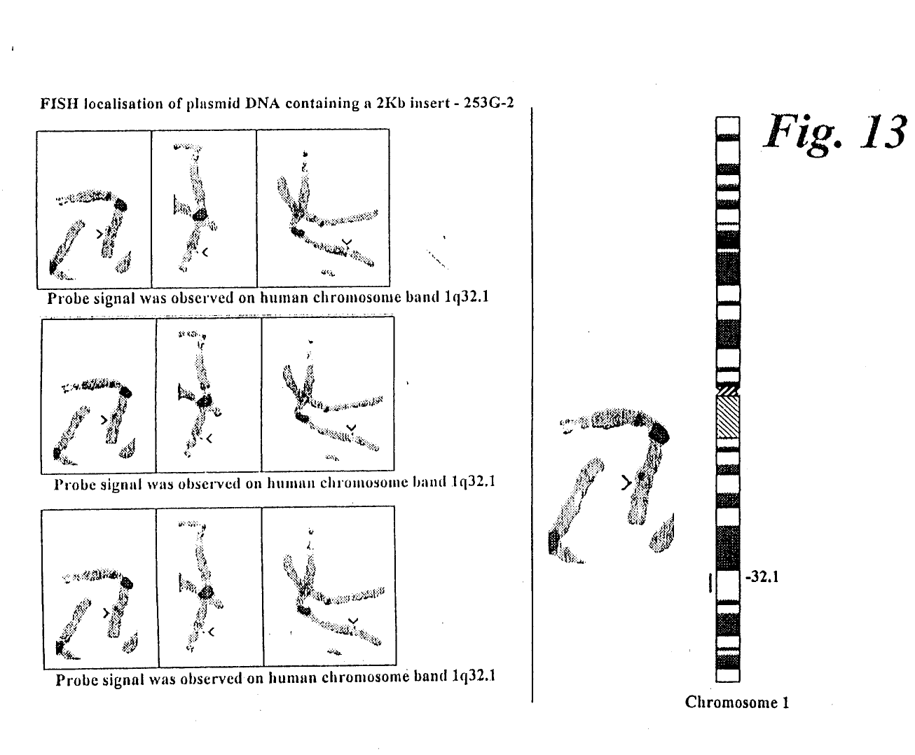

FIG. 13 shows the chromosomal location of the plu-1 gene as human chromosome band 1q32.1. The plasmid clone 253G-2 was used as a probe (see above). Fluorescent in situ hybridisation (FISH) was performed and the probe was detected with one round of avidin-fluoroisothiocyanate (FITC). At least 20 cells were examined. Hybridisation efficiency was low (only approximately 50% of cells examined showed a signal) because the insert size was small at approximately 2 kb. However, the signal was small and discrete and could be localised to human chromosome band 1q32.1.

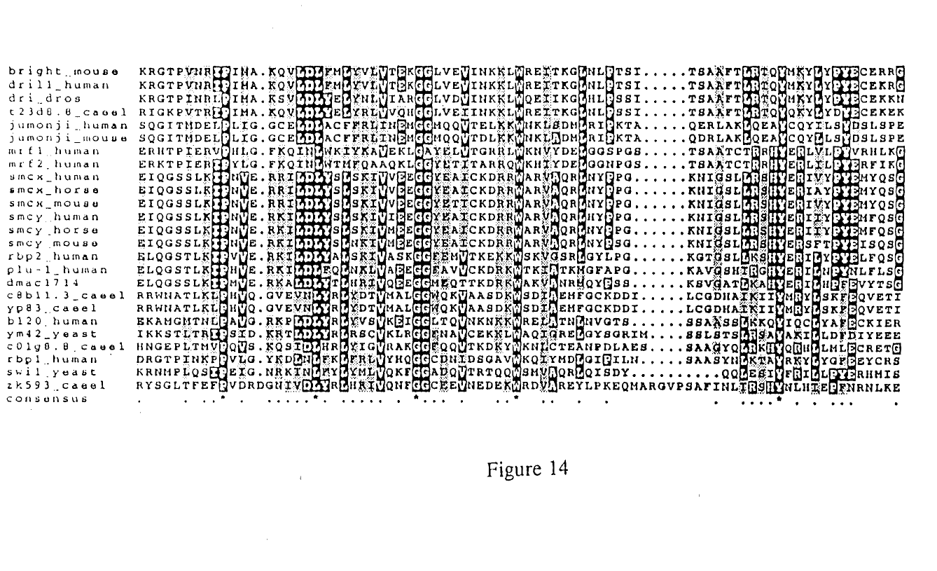

FIG. 14 shows an alignment of the conserved DRI (dead ringer) domain within plu-1 and related proteins. The sequences are listed in descending order of overall similarity, the following list provides the amino acid residues ranges and appropriate database accession number for each protein:

bright_mouse (259-336; TREMBL:Q62431)

drill_human (254-331; TREMBL:Q99856)

dri_dos (296-374; TREMBL:Q24573)

t23d8.8_caeel (23-100; TREMBL:O002326)

jumonji_human (637-714; TREMBL:Q92833)

jumonji_mous (635-712; Swissprot:Q62315)

mrf1_human (91-168; TREMBL:Q03989)

mrf2_human (33-110; TREMBL:Q14865)

smcx_human (94-170; Swissprot:P41229)

smcx_horse (59-135; TREMBL:P79352)

smcx_mouse (59-132; Swissprot:P41230)

smcy_human (94-170; TREMBL:Q92809)

smcy_horse (59-132; TREMBL:P79353)

smcy_mouse (72-151; TREMBL:Q62240)

rbp2_human (99-175; Swissprot:P29375)

plu-1_human (112-188)

dmac1714 (87-163 EMBL:AC001714)

c8b11.3_caeel (40-117; Swissprot:Q09441)

yp83_caeel (40-117; TREMBL:Q09441)

b120_human (650-726; TREMBL:D1024146)

ym42_yeast (202-280; Swissprot:Q03214)

c01g8.8_caeel (278-356; TREMBL:P91019)

rbp1_human (325-402; TRENIBL:P29374)

swil_yeast (422-494; Swissprot:P09547)

zk593_caeel (136-219; TREMBL:Q23541)

The sequences were aligned using ClustalX, with gaps indicated by full stops. The consensus line and shading were created with BoxShade 3.2, conserved residues are shown as white on black, conservative substitutions are indicated as black on grey. The consensus line shows conservative substitutions as full stops and conserved positions as asterisks.

FIG. 15 shows the results of in situ hybridisation of breast tissue using a plu-1 probe. FIG. 15(a) (45-96C (human breast grade 1 ductal tumour)) shows increased plu-1 MRNA in invasive tissue. FIG. 15(b) (199 96C (human breast grade 3 ductal tumour)) shows presence of low levels of plu-1 mRNA in a cyst, increased levels of plu-1 mRNA in a DCIS region, and further increased levels of plu-1 mRNA in invasive tissue. For each pair of tissue sections the top panel has been stained with Giemsa, while the bottom panel has been processed for in situ hybridisation using the 253g2 clone as a probe.

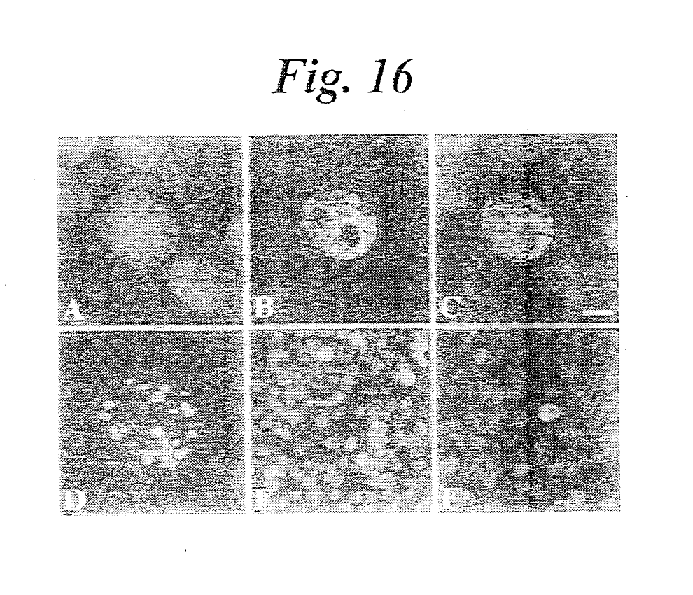

FIG. 16 shows nuclear localisation of the plu-1 gene product. Cos cells were transiently transfected with the myc-tagged plu-1 gene and stained with the 9E10 antibody and or DAPI after 3 days (panels A-E). At three days, the G418 selectable marker was added and the cells stained 17 days later (panel F). Staining with DAPI (A), with Ab 9E10, or with both reagents (C) illustrates the nuclear but not nucleolar localisation of the plu-1 product.

An individual cell stained with the 9E10 antibody (D) was analysed by confocal microscopy and a composite image assembled demonstrating the presence of the tagged plu-1 product in discrete foci in the nucleus.

Loss of expression of plu-1 with time after transfection and selection is illustrated by comparing panels E and F, both stained with DAPI and 9E10.

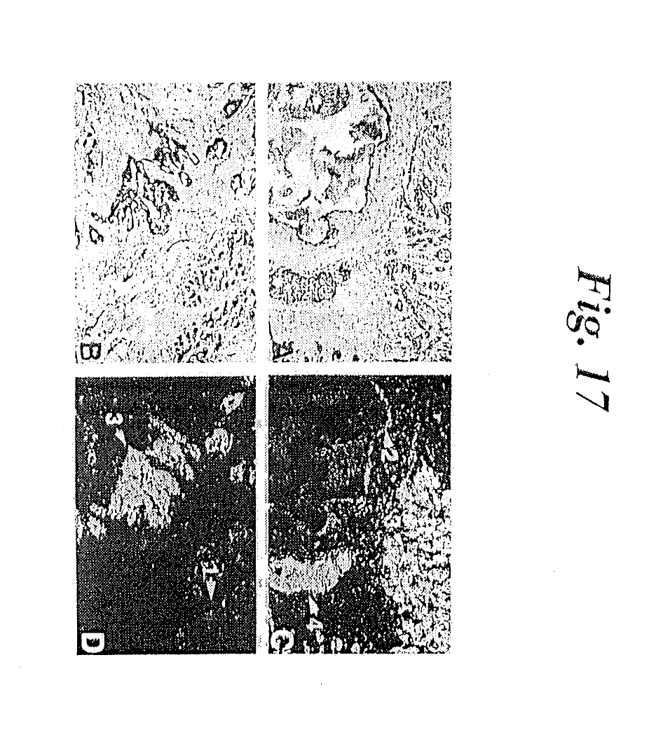

FIG. 17 shows in situ analysis of plu-1 mRNA expression in a Grade 3 ductal carcinoma (A,C), and in a grade I ductal carcinoma. Paired light and dark field photomicrographs of tumour sections hybridized with a plu-1 riboprobe. In light field illumination reduced silver over the hybridized mRNA is seen as a black deposit (AB), whilst under dark field illumination the silver appears white (C,D). Invasive grade III ductal carcinoma of the breast (A,B→3) shows a very strong signal for plu-1 mRNA as does ductal carcinoma in situ [DCIS→4]. There is a weak signal over epithelium lining the large cyst (→2) representing attenuated DCIS epithelium. Invasive grade I ductal carcinoma shows a strong signal for plu-1 mRNA (C,D→3) in contrast to the weaker signal over the benign acini (→1), particularly when remote from the malignant tissue.

### EXAMPLE 1

### Isolation and Identification of the plu-1 cDNA and its Relationship to the Breast

Isolation of a Partial cDNA Coding for the plu-1 Gene

The breast epithelial cell line MTSV1-7 developed from cultured human milk epithelial cells (Bartek et al (1991) PNAS 88, 3520-24) was transfected with the c-erbB2 oncogene (D'Souza et al (1993) Oncogene 8, 1797-1806). Such cells exhibit a similar phenotype to breast cancer cells. The transfected cell line (cel) was treated for 2 days with an antibody to down regulate the phosphorylation of the c-erbB2 and thus inhibit signalling. cDNAs were prepared from mRNA isolated from the untreated cel cells, and the cel cells treated with antibody, and these cDNAs were used as probes to screen a foetal brain library.

A clone (23G2) was isolated which, in northern analysis, bound to a band of approximately 6 kb expressed at high levels by cel cells, but not by the parental MTSV1-7 cell line. The level of the 6 kb mRNA was reduced in cel cells treated with the antibody.

As is described in more detail below, several ESTs in the data base showed homology with the 23G2 sequence.

Translation in one reading frame showed homology with the RBP-2 (retinoblastoma binding protein-2) gene but the LFCDE (LxCxE) sequence in the encoded polypeptide, believed to be required for RB binding was not present. Homology with the huxe169 gene was also noted.

Isolation of Full Length cDNA Sequence

Further sequence for the plu-1 gene was obtained by screening a library from the breast cancer cell line ZR75.

The full length cDNA nucleotide sequence is shown in FIG. 1, and the amino acid sequence is in FIG. 2. Homologies with other genes and known ESTs are shown in FIGS. 3 to 7.

Expression of the plu-1 Gene

Using the 25G2 probe the plu-1 gene was seen to be expressed in all breast cancer cell lines examined (FIG. 9) by northern analysis, but not in colon cancer cell lines (FIG. 10). There appears to be no correlation between the level of expression of c-erbB2 and the level of expression of plu-1. Expression was also seen in two early cultures of a primary breast cancer.

Using in situ analysis the plu-1 gene was shown to be expressed in primary breast cancers, but not in colon cancers. Normal adult tissues were examined by northern analysis and plu-1 was found to be expressed at high levels only in testis, with low levels being detected in placenta ovary and tonsil.

FIGS. 8 to 11 show various northern blots.

The plu-1 gene has been located on chromosome 1q32.1.

### CONCLUSION

The sequence of a gene (plu-1) has been obtained which appears to be overexpressed in breast cancers, and which is normally silent in most adult tissues. The gene has at least two potential applications:

as a marker for breast cancer

as a target antigen for the immune system in immunotherapy of breast cancer.

Materials and Methods

Cell Culture

Cell lines: MTSV1-7, cel, T47D, and ZR75 cells were grown in DMEM supplemented with 10% FCS (Gibco) and 0.3 μg/ml glutamine. This medium was supplemented with 5 μg/ml of hydrocortisone (Sigma) and 10 μg/ml of insulin (Sigma) for MTSV1-7 cells and ce-1. For ce-1 cells, the selectable marker G418 (Gibco) was also added at a concentration of 500 μg/ml. The SKBR-3 and MCF-7 cells were grown in RPMI containing 3.7% bicarbonate, 10% FCS (Gibco) and glutamine. The same medium with added insulin was used for MCF-7. The BT20 cell line was maintained in MENBic with 15% FCS plus insulin and glutamine.

Culture of Primary Breast Carcinomas: Two samples of invasive breast carcinomas (numbers 4 and 9) provided by the Hedley Atkins/ICRF Breast Pathology Group at Guy's Hospital were cut into 1.2 mm³ sections and digested with 20 ml of collagenase (Sigma) at 450 units/ml in E4 10% FCS overnight on a rotary shaker. After washing with E4 in decreasing concentrations of FCS (10, 5, 2%o), the cells were grown in 1.05 mM Ca⁺⁺ E4/F12 (1:1) supplemented with 2% FCS depleted of Ca⁺⁺ and growth factors. After 1-2 days the medium was replaced with medium of identical composition but with lower Ca⁺⁺ (0.06 mM) (Shearer et al (1992) Int. J. Cancer 51, 602-612). Cultures were passaged by trypsinization and total cellular RNA was extracted from tumour number 4 at passage 2. Cells from tumour number 9 were transduced with the bcl-2 gene using a recombinant retrovirus (Lu et al (1995) J. Cell Biol. 129, 1363-1378), and RNA was extracted at passage 11.

Isolation of cDNA Coding for the Novel plu-1 Gene

Isolation of the first partial clone: ce-1 cells were grown to approximately 50% confluence and then grown for 48 hrs in the presence or absence of 50 ng/ml of an antibody which inhibits phosphorylation of c-erbB-2 on tyrosine residues. Poly A+RNA was isolated from total RNA from the treated and untreated cells using oligo (dT) chromatography according to the poly A Quik kit (Stratagene), then converted to CDNA using the Superscript II reverse transcriptase (Gibco). The cDNAs were subsequently used as probes labelled with [α-³³P] dCTP by random priming.

Filters carrying 10⁵ clones from a cDNA library made from human foetal brain were hybridized with the above labelled probes. The labelling was evaluated by computerized analysis with a phosphorimager. Differentially expressed clones were selected and expression verified by northern blot of the cel cells. Analysis of 7 clones, demonstrated a novel sequence in clone 253G2 which gave a weaker signal with the probe from the antibody treated cells.

Isolation of clones covering the full plu1 gene: For isolation of further sequences of the gene containing the 253G2 sequences, 3 cDNA libraries were used, namely a ZR75 phage library, a Jurkat plasmid library and a testis phage library. The cDNA library from the human breast carcinoma cell line ZR75 was oligo/dT primed and cDNA sequences were cloned into the uni-ZAP XR vector (Stratagene) with XhoI at the 3′ and EcoR1 at the 5′ end (Cavailles et al (1995) EMBO J. 14, 3741-3751). 106 plaques from the ZR75 library were screened initially using a fragment of 253G2-sequence and subsequently with 5′ sequence obtained from the longer clones. Three consecutive screenings were performed and 22, 27, and 12 plaques picked respectively from the original plates. The plaques containing the largest clones with most 5′ sequence were determined by toutdown and semi-nested PCR on the original plaques. Plaques were then purified by secondary and tertiary screens and pBS-SK(−) plasmids obtained by in vivo excision.

Since the 5′ end of the gene was not obtained in the three screens of the ZR75 library, a Jurkat cDNA library was screened. This library was prepared by priming cDNA from the human T-leukemia cell line J6 with random hexamers (Dunne et al (1995) Genomics 30, 207-223). The whole library was screened by PCR using a sequence from the ZR75 clones containing the most 5′ sequence. The PCR product was purified using a JET-sorb DNA Extraction kit and sequenced. 450 bp of new 5′ sequence was thus obtained and used as a probe for a 4^{th} screen of the ZR75 library from which the clone 1.2 was isolated. 280 bp of sequence was covered by only one clone (between consensus sequence 665-937). This piece of the sequence was further confirmed by screening a human Testis 5′-STRETCH PLUS cDNA Library from Clontech and isolating clones covering the sequence. Sequencing was performed using an ABI Prism Dye Terminator Automated cycle Sequencer.

The entire sequence was obtained from at least two individual clones covering the same region and both were sequenced in each direction. Analysis of the consensus cDNA sequence revealed a single long ORF of 4635 nucleotides, starting at position 90 and ending with a TAA termination codon at 4724. The sequence encodes a 1545 amino acid protein with a predicted size of 170 KD. The untranslated 3′ is 1569 nt, and contains a terminal polyA region of 65 As.

Assembly of Full Length plu1 cDNA

Construction of the full length plu-1 cDNA: Three overlapping clones (ZR75 1.2, 3.1 and 14) containing unique restriction enzyme sites were used for construction of the full length cDNA. The most 5′ clone (clone 1.2) in the Bluescript plasmid was cut with Bgl II/XhoI at base 466 and at the 3′ end cloning site respectively, leaving the 5′ 466 bp in the plasmid vector. The 2^{nd} clone (3.2) was digested with BglII/Avr II and the 2435 bp fragment isolated. The 3′ clone (14) was cut with Avr II/Xho I and the 3476 bp fragment comprising the rest of the 3′ sequence was separated. The 3 purified fragments were joined together in one reaction with T4 DNA ligase. The recombinant clones were sequenced over the join regions, and the final construct with a 6.4 kb insert is referred to as pBS-SK(−)/plu1.

Development of plu1 cDNA with Myc-His tag: Based on the analysis of the restriction enzymes in the sequence and the amino acid coding sequence, the mammalian expression vector pcDNA 3.1 (−)/Myc-His A (Invitrogen) with a C-terminal Myc-His tag driven by the CMV promoter was selected for constructing the tagged gene. A 3′ plu-1 coding fragment (632 bp) was generated by PCR where, at the 3′ end, the TAA stop codon was replaced to give an Xho I site flanked by a HindIII site. [The HindIII site at the 3′ end allowed the cloning into the pcDNA vector, while the Xho site allowed the whole plu-1 sequence to be retrieved if required]. The 3′ sequence on the coding strand generated by the p3HindIII antisense primer is aligned below with the wild type sequence.

GAC GCA CCA AGC CGA AAG TAA AAA CAC AAA AAC AGA

(WT)

GAC GCA CCA AGC CGA AAG CTC GAG AAG CTT AAC AG
Xho I HindIII

The 5′ primer included an NcoI site to link the PCR fragment to the rest of the plu-1 sequence which was excised as a 4106 bp XbaI/NcoI fragment from the pBS-SK(−)plu-1 construct. The PCR product, (cut with NcoI and HindIII) the Xba/NcoI fragment and the pcDNA 3.1 Myc-HisA vector, linearized with XbaI and HindIII were then ligated in one reaction. The recombinant clones were sequence over the joins and PCR regions and the final construct with a 4.781 kb insert is referred as plu1-ORF/Myc-His A.

Transient Transfection

Electroporation: The expression of the recombinant protein with the tagged plu1-ORF/Myc-His A construct was first checked by transient expression of Cos cells. The cells were grown to 70% confluence, trypsinized, washed with PBS, and 5×10⁶ cells resuspended in 1 ml PBS with 20 μg DNA either from the Myc-His construct of the empty vector as control. The cells were electroporated with a Gene pulser (Bio Rad) using 250 μF with 450V and then resuspended in 30 ml growth medium and plated on 9 cm dishes and glass cover slips for western blot analysis and immunostaining.

Calcium phosphate mediated transfection: Breast cancer cell lines (T47D, MCF-7, BT.20, ZR.75 and the HT1080 cell line) were grown on 3 cm dishes to approximately 60% confluence and transfected directly with the calcium phosphate coprecipitate overnight as previously described (D'Souza et al (1993) Oncogene 8, 1797-1806). Two to three days after the removal of the DNA precipitates, cells were used for indirect immunofluorescent staining.

Immunofluorescent Staining

Cells on cover slips or 3 cm dishes were washed with PBS, fixed with 4% paraformaldehyde for 15 minutes, and permeabilized with 0.1% Triton for 5 min. After blocking with 20% FCS/PBS for 30 min, cells were incubated with the 9E10 mAb to the Myc tag (10 ug/ml) and then with FITC conjugated rabbit anti-mouse Ig 1:50 (Dako).

Western Blot Analysis

The level of inhibition of tyrosine phosphorylation of the c-erbB2 gene product with the c-erbB2 antibody, and the expression of the Myc-tagged plu-1 gene product from transiently transfected Cos cells was assessed by subjecting 100 μg of total lysates to immunoblot analysis with the respective Abs.

Confluent ce-1 cells, (treated or not treated with c-erbB2 Ab) in 9 cm tissue culture dishes were washed three times with cold phosphate-buffered saline (PBS) containing 1 mM sodium orthovanadate and then lysed with 1 ml of lysis buffer (D'Souza et al (1993) Oncogene 8, 1797-1806). For detection of the recombinant Myc-tagged plu1 gene product, the transiently transfected cos cells were lysed in HNET buffer (50 mM Hepes, pH 7.5, 100 mM NaCl, 1 mM EGTA, 1% Triton X-100, 1 nMDTT, and 1 mMPMSF). After clarification of the lysates by centrifugation at 15,000 g for 10 min at 4° C. the protein concentration of the lysates was estimated using the Bio-Rad protein assay kit. Samples were then electrophoretically separated on a 5% stacking/7.5% running SDS-PAGE, and transferred to Hybond-C membrane (Amersham).

Immunoblots were blocked with 3% BSA or 5% skimmed milk/0.1% Tween-20 in PBS for 2 hrs, probed with antiphosphotyrosine mAb PY20, 1:100 (Upstate Biotechnology) or 1 μg/ml anti-Myc mAb, 9E10, for 2 hrs. The immune complexes were detected with ¹²⁵I-labelled sheep anti-mouse Ig 0.5 μci/ml (Amersham) for PY20 or peroxidase-conjugated rabbit anti-mouse 1:2000 (Dako) for 1 hr. The band was developed using the enhanced chemiluminescence detection kit (Amersham).

Northern Analysis of RNA from Cell Lines and Strains

Total cellular RNA from the cell lines or cultures of primary breast cancers was isolated according to the method of Chomczynski and Sacchi (1987) Anal. Biochem. 162, 156-159. The total cellular RNA of the colon cancer cells was a kind gift from Helga Durbin. 20 μg RNA from each cell type was denatured in 1×Mops, 0.66M formaldehyde and 50% (vol/vol) formamide, and subsequently size fractionated on a 1.2% agarose-formaldehyde gel. The RNA was transferred and immobilized onto Hybond-N (Amersham). The membrane-bound RNAs were hybridized with the ³²P dCTP cDNA probes labelled by random priming, and washed to high stringency according to the protocol of Church and Gilbert (1984). The 1.97 kb NotI/SalI cDNA fragment from the initial clone 253G2 (3′plu1) was used for detecting plu1 mRNA, and the 4.4 kb HindIII fragment of the pSV2-erbB2 for c-erbB2 mRNA. To assess the efficiency of loading and transfer of the RNA, the membranes were reprobed for GAPDH expression. The Human Normal Blots I, II, III (FIG. 11) carried total RNA from 24 normal adult tissues, 8 on each blot (Invitrogen), and the control β-actin probe was provided.

Fluorescence in situ Hybridisation (F7SH) of plu1 for Chromosomal Localisation

30 metaphase spreads prepared from phytohaemaglutinin-stimulated normal human lymphcytes by standard techniques were analysed. Before hyridisation the slides were denatured in 70% formarnmide and 2×SSC at 73° C. for 3 minutes, washed in 2×SSC and dehydrated through an ethanol series of cold 70%, 95% and absolue ethanol. Probe DNA (either 253G2, or the full length sequence from BS-SK(−) plu-1) was biotinylated using the Bionick kit (Gibco BRL). 500 ng of labelled probe was mixed with 5 μg Cot-1 DNA (Gibco BRL) precipitated, resuspended in 11 μg hybridisation mix, denatured at 85° C. for 5 minutes and allowed to preanneal at 37° C. for 30 minutes. After preannealing, the probe was applied to a denatured slide and hybridised at 37° C. overnight.

Slides were washed in 50% formamide, 2×SSC pH 7.0 at 42° C., followed by 1×SSC at 60° C. Blocking solution (3% BSA, 4×SSC and 0.1% Tween 20) was applied and slides incubated at 37° C. for 30 minutes. After incubation, avidin-FITC (diluted in 1% BSA, 4×SSC, 0.1% Tween 20) was applied and slides incubated at 37° C. for 40 minutes. Slides were washed in 4×SSC, 0.1% Tween 20 at 42° C. and counterstained with DAPI (4, 6-diamidino-2-phenylindole 200 ng/ml), followed by 2 minutes in 2×SSC. Slides were mounted in Citifluor and images captured using a Photometrics KAF 1400-50 CCD camera attached to a Zeiss Axioskop epifluorescence microscope. Separate images of probe signals and DAPI banding patterns were pseudocoloured and merged using SmartCapture software (Vysis, Inc, Chicago, Ill., USA). In all the spreads a signal was observed on both copies of chromosome 1 band 1q321. No other consistent signal was observed.

Results

Isolation of the plu1 Gene

The MTSV1-7 cell line was derived by immortalisation of luminal epithelial cells cultured from human milk (Bartek et al (1991) Proc. Natl. Acad. Sci. USA 88, 3520-3524) and the ce-I cell line was developed by transfection of MTSV1-7 with c-erbB2 cDNA (D'Souza et al (1993) Oncogene 8, 1797-1806). To look for genes whose expression is regulated by signals generated through c-erbB2, phosphorylation of the receptor was down regulated by treatment with the c-erbB2 antibody for 48 hours. The cDNAs, prepared from MRNA from cel cells treated or not treated with antibody, were then used as labelled probes to differentially screen a foetal brain library. The clone 25G2, which showed a weaker signal with cDNA from the antibody treated cells, was identified and the insert sequenced. Translation of the open reading frame gave an amino acid sequence which showed strong homology with the RB binding protein RPP-2 (Defeo-Jones et al (1991) Nature 352, 251-254; Fattaey et al (1993) Oncogene 8, 3149-3156). Using 5′ sequences from the 25G2 clone further clones covering and extending the 25G2 sequence were isolated from a cDNA library prepared from a breast cancer cell line (ZR 75). Further screens of the breast cancer library were required to obtain the full sequence and three overlapping clones were assembled (as described in Materials and Methods) to give a full length cDNA region (see FIG. 1).

Homologies with Other Genes

FIG. 3 shows the translated open reading frame, optimally aligned to other genes in the data base showing homology and FIG. 1B summarised the data diagrammatically. The strongest homology was seen with a human RB binding protein RBP-2, particularly in the first 200 amino acids and over a large domain beginning around amino acid 308. This domain has seven conserved cysteines within the first 50 amino acids and there is extensive conservation of aromatic amino acids (6 tryptophans, 5 tyrosines) as well as basic and hydrophobic residues. There is, however, no known function identified with this region. The RB binding motif LXCXE found in RBP2 was not found in plu-1. Homology to these same regions is found in other human genes, including the humx169a gene which is found on the X chromosome but which is not inactivated (Wu et al (1994) Hum. Mol. Genet. 3, 153-160) and which shows 90% homology to sequences found and expressed on the Y chromosome (Agalnik et al (1994) Hum. Mol. Genet. 3, 879-894). Another gene, KIAA (Nagase et al (1996) DNA Res. 3, 321-329) shows a stronger homology with the humx169a and hssmcy genes than with plu-1.

The two domains in plu-1 also exhibit strong homology with sequences found in other organisms. The mouse gene homologous to the human 169a gene represents the first gene on the mouse X chromosome reported to escape inactivation. The functions of most of the homologous genes, including those in C. elegans, Drosophila and S. cerevisiae, have not been defined (for accession numbers see legends to FIGS. 3 and 4).

The domain at the 5′ end contains a DNA-binding motif found in several known genes and previously reported in the dead ringer (dri) drosophila gene (Gregory et al (1996) Mol. Cell. Biol. 16, 792-799). The sequence from dead ringer, when expressed, has been shown to bind the same DNA sequence in vitro as the engrailed (which contains a classic homeodomain), even though dri and engrailed show no homology. The dri motif is found in a large number of genes, many of which do not show extensive homology to plu-1. The members of this family may be important in the regulation of genes related to particular cell phenotypes.

Other motifs of interest present in plu-1 are 3 PHD domains which are zinc binding domains thought to be involved in transcription, and 3 nuclear import signals. Together with the homology to the dri motif, these sequences suggest that the plu1 gene product is a nuclear protein, possibly involved in transcriptional control.

Cellular Localisation of the plu1 Gene Product

To determine the intracellular location of the protein, the cDNA was tagged with a myc epitope recognised by the antibody 9E10 and transiently transfected into Cos cells. Western blot analysis of extracts of the transfected cells using the anti-myc antibody showed detected a single band of the expected size (170 kDa). Immunohistochemical staining of the transfected cells 3 days after transfection showed unambiguously that the protein was localised to the nucleus, but not the nucleolus (FIGS. 16A-C) FIG. 16D, representing a composite image from con-focal microscopy, shows that the staining of the tagged gene product is clearly associated with discrete foci in some of the cells. Similar patterns of staining were obtained in transient transfections of other cell lines (-breast cancer cell lines T47D, MCF7, ZR-75, BT20, MTSV1-7, and two non epithelial cell lines HT1080 and HB96, data not shown). The pattern of nuclear foci shown by plu-1 was compared to that shown by two sn RNP recognised by the antibodies Y12 and SC35. The larger number of foci seen with plu-1 was also noted with antibody SC35, while only 1-3 foci were seen in the Y12 stained nuclei (data not shown).

The plu 1 Gene is Specifically Expressed in Breast Cancers

The original sequences isolated in the clone 253G2 were used to examine expression of plu-1 mRNA by Northern analysis. The 253G2 clone contains some translated sequence together with untranslated sequence all of which shows little homology with the other human genes and therefore this probe should detect only plu-1 RNA. FIG. 8 shows that the level of expression of plu-1 mRNA in ce-1 cells decreases after treatment with the anti-c-erbB2 antibody which strongly inhibits phosphorylation of c-erbB2.

To evaluate expression of plu-1 in primary breast cancers more fully, in situ hybridisation was performed using the 253G2 probe and sections of breast cancers and benign lesions. Fifteen malignant tumours were examined (4 Ductal Grade 1, 4 Ductal Grade 2, 4 Ductal Grade 3 and 3 lobular carcinomas). In all the ductal carcinomas and in 2 of the 3 lobular carcinomas, the invasive component showed strong staining for plu-1, with the Grade 3 Ductal tumours showing the highest level of expression. In situ components also showed strong staining with the 253G2 probe, while benign components of the carcinomas were negative or weakly positive except when closely bordering the invasive component, when the labelling became stronger. Fibroadenomas (3) and lactating adenomas (2) showed only a weak signal with the plu-1 probe. FIG. 17 shows examples of staining of invasive, in su, and benign components of a grade 1 and a grade 3 ductal carcinoma. Although the numbers are small, the results suggest that plu-1 expression is upregulated in breast cancers but not in benign lesions and within the tumours, the highest expression is seen in the invasive component.

Restricted Expression of plu-1 in Normal Adult Tissues

To assess the expression of plu-1 in normal adult tissue, Northern blots of mRNA from a range of tissues were probed with the 25G2 probe. FIG. 11 shows that the only tissue showing a high expression of plu-1 is testis, although low levels of-expression of mRNA were detectable in placenta, ovary and tonsil. Apparently expression of plu-1 is highly restricted in normal adults, which distinguishes it from the homologous RBP-2 and humxe169a genes, reported to be ubiquitously expressed (Fattaey et al (1993) Oncogene 8, 3149-3156; Wu et al (1994) Genetics 3, 153-160). The chromosomal location of plu-1 also distinguishes it from the homologous genes as it is located on chromosome 1q32.1 as shown in FIG. 13.

### EXAMPLE 2

### Production of Activated Cytotoxic Lymphocytes (CTL) Using Class I Molecules and plu-1 Antigen and Their Administration

Activated cytotoxic T lymphocytes (CTLS) are produced using HLA-A2 Class I molecules and any of the plu-1 peptide antigens listed in FIG. 12.

In particular, any of the 9-mer peptides starting at positions 711, 906, 1058 and 1338 in the plu-1 polypeptide sequence are used.

The method described in PCT patent application WO 93/17095 is used to make the CTLs. Drosophila cells are used to present the peptide antigen to CTL. The HLA-A2 molecule is expressed in the Drosophila cells.

Antigenic plu-1 peptides are obtained from naturally-occurring sources or are synthesised using known methods. For example, peptides are synthesised on an Applied Biosystems synthesiser, ABI 431A (Foster City, Calif., USA) and subsequently purified by HPLC.

As is described in detail in WO 93/17095, in order to optimize the in vitro conditions for the generation of specific cytotoxic T cells, the culture of stimulator cells is maintained in an appropriate medium. The stimulator cells are Drosophila cells as described in WO 93/17095, which are preferably maintained in serum-free medium (eg Excell 400).

Prior to incubation of the stimulator cells with the cells to be activated, eg precursor CD8 cells, an amount of antigenic peptide is added to the stimulator cell culture, of sufficient quantity to become loaded onto the human Class I molecules to be expressed on the surface of the stimulator cells. A sufficient amount of peptide is an amount that will allow about 200, and preferably 200 or more, human Class I MHC molecules loaded with peptide to be expressed on the surface of each stimulator cell. The stimulator cells are typically incubated with >20 μg/ml peptide.

Resting or precursor CD8 cells are then incubated in culture with the appropriate stimulator cells for a time period sufficient to activate the CD8 cells. The CD8 cells shall thus be activated in an antigen-specific manner. The ratio of resting or precursor CD8 (effector) cells to stimulator cells may vary from individual to individual and may further depend upon variables such as the amenability of an individual's lymphocytes to culturing conditions. The lymphocyte:stimulator cell (Drosophila cell) ratio is typically in the range of about 30:1 to 300:1. For example, 3×10⁷ human PBL and 1×10⁶ live Drosophila cells are admixed and maintained in 20 ml of RPMI 1640 culture medium.

The effector/stimulator culture are maintained for as long a time as is necessary to stimulate a therapeutically usable or effective number of CD8 cells. The optimum time is typically between about one and five days, with a “plateau”, ie a “maximum” specific CD8 activation level, generally being observed after five days of culture. In vitro activation of CD8 cells is typically detected within a brief period of time after transfection of a cell line. Transient expression in a transfected cell line capable of activating CD8 cells is detectable within 48 hours of transfection. This clearly indicates that either stable or transient cultures of transformed cells expressing human Class I MHC molecules are effective in activating CD8 cells.

Activated CD8 cells may be effectively separated from the stimulator (Drosophila) cells using monoclonal antibodies specific for the stimulator cells, for the peptides loaded onto the stimulator cells, or for the CD8 cells (or a segment thereof) to bind their appropriate complementary ligand. Antibody-tagged molecules are then extracted from the stimulator-effector cell admixture via immunoprecipitation or immunoassay methods.

Effective, cytotoxic amounts of the activated CD8 cells can vary between in vitro and in vivo uses, as well as with the amount and type of cells that are the ultimate target of these killer cells between about 1×10⁶ and 1×10¹² activated CTL are used for adult humans, compared to between about 5×10⁶ and 5×10⁷ cells used in mice.

The activated CD8 cells are harvested from the Drosophila cell culture prior to administration of the CD8 cells to the individual being treated. It is important to note, however, that unlike other present and proposed treatment modalities, the method described in this Example uses a cell culture system (ie Drosophila cells) that are not tumorigenic. Therefore, if complete separation of Drosophila cells and activated CD8 cells is not achieved, there is no inherent danger known to be associated with the administration of a small number of Drosophila cells, whereas administration of mammalian tumor-promoting cells may be hazardous.

Methods of re-introducing cellular components are used such as those exemplified in U.S. Pat. No. 4,844,893 to Honsik et al and U.S. Pat. No. 4,690,915 to Rosenberg. For example, administration of activated CD8 cells via intravenous infusion is appropriate.

### EXAMPLE 3

### Dendritic Cells Pulsed with plu-1 Peptide for Treating Breast Cancer

Any of the 9-mer peptides starting at positions 711, 906, 1058 and 1338 in the plu-1 polypeptide sequence are used.

Breast carcinoma is potentially curable only when truly localised. The most common problem is either late presentation with overt metastases or, more frequently, the development of systemic metastases after apparent local cure. Metastatic breast carcinoma is highly chemosensitive and effective chemotherapy routinely induces disease remission, allowing delay in the onset of secondary disease or amelioration of the symptoms of extensive disease.

Adoptive immunotherapy is based on the proposition that tumour growth and dissemination reflects a failure in immunological surveillance, either due to reduction in antigen presentation by the neoplastic cells or due to generalised decline in patient immunity. There is evidence that both mechanisms occur in breast carcinoma and in particular that there are important deficiencies in dendritic cell (DC) function (Gabrilovich et al (1997) Clin. Cancer Res. 3, 483-490). Cytotoxic T cell responses are demonstrated in vitro to immunogenic peptides such as plu-1. DC are professional antigen-processing and presenting cells which are critical to the development of primary MHC-restricted T-cell immunity. They originate from a CD34⁺ precursor in bone marrow, but can also be derived from a post colony-forming unit CD14⁺ intermediate in the peripheral blood. DC migrate to peripheral sites in skin, mucosa, spleen and thymus. They have been implicated in a variety of clinically important processes, including allograft rejection, atopic disorders, autoimmunity and anti-tumour immunity.

The patient is typed as HLA-A2.

DC are cultured ex vivo from CD34⁺ stem cells or CD14⁺ peripheral blood monocytes using cytokines, principally GM-CSF, IL-4 and TNFα. DC from both these sources are immunocompetent and can take up exogenously presented antigen, process it and then present it to cytotoxic T-cells (Grabbe et al (1995) Immunology Today 16, 117-121; Girolomoni & Ricciardi-Castagnoli (1997) Immunology Today 18, 102-104). Recent studies have demonstrated that DC can transfer antigen-specific tumour immunity generated in vivo (Kwak et at (1995) Lancet 345, 1016-1020) and that autologous DC pulsed with tumour antigen ex vivo can induce a measurable anti-tumour effect (Hsu et al (1996) Nature Medicine 2, 52-58). DC can be effectively pulsed using a crude tumour membrane lysate, purified peptides or peptide fragments.

Plu-1 is a polypeptide expressed by breast cancers. Although plu-1 is expressed by normal cells, adenocarcinomas display alterations in intensity of expression.

Keyhole limpet haemocyanin (KLH) is an immunogenic protein which is used as an innocuous positive control for the immunocompetence of the patient in studies similar to this (Hsu et al (1996) Nature Medicine 2, 52-58).

The feasibility of using ex vivo expanded autologous dendritic cells from patients with recurrent breast carcinoma, loaded with a purified preparation of the tumour antigen plu-1 and reinfused as adoptive immunotherapy, is established in the following way.

The work described establishes optimal methodology for the generation of autologous DC by ex vivo expansion from peripheral blood of patients with recurrent breast carcinoma; assesses the feasibility of loading DC with exogenous peptides plu-1; examines acute tolerability and toxicity of autologous reinfusion; examines whether an immune response to plu-1 or KLH develops; and examines the effect on measurable tumour bulk.

Adoptive immunotherapy is likely to prove most effective in the control or elimination of minimal residual disease rather than in the reduction of bulk disease. It is conceivable that immunotherapy may temporarily increase the dimensions of bulk disease due to influx of cytotoxic T lymphocytes. Extent and bulk of disease will be monitored following therapy but not used as a formal endpoint. Patients are followed up in the routine manner in the long term to ensure that no long term adverse events are manifest.

Dendritic Cell Culture from Normal Volunteers

CD14⁺ peripheral blood monocytes are adhered to tissue culture flasks and cultured in the presence of 1% AB serum, GM-CSF (400 ng/ml) and IL4 (400 IU/ml) for 7 days. This yields cells with the morphology of DC and a mean of 49% with the CD1a⁺ marker which is indicative of the immature form of the DC capable of taking up and presenting antigen. These cells are then matured to CD83⁺ cells by the addition of TNFα (15 ng/ml), which enables the DC to present antigen to cytotoxic T-cells. 7% of the cells become CD83⁺ within 1 day, but 3 days at least are required for maximum effect. It is possible that monocyte conditioned medium could replace the 1% AB serum.

Dendritic Cell Culture from Patients with Relapsed Breast Carcinoma

DC are generated from 6 patients with relapsed metastatic disease, both prior to and following salvage chemotherapy (a total of 12 samples of peripheral blood, each of 50 mls).

Clinical Study

Patients donate a single unit of autologous blood according to standard protocol. Patients are evaluated prior to donation by a blood transfusion service physician. Autologous donations are screened in the same way as allogeneic donations for routine virus markers (HIV, HBV, HCV and syphilis) and patients give consent to this after appropriate counselling if they wish to participate. This precaution protects clinical and laboratory staff from potential infection and the routine blood supply from the possibility of cross-contamination. The blood is taken into a routine quad-pack. This allows automated separation of red cells, buffy coat and plasma. The buffy coats yields approximately 670×10⁶ mononuclear leukocytes which give approximately 47×10⁶ DC using current techniques. A dosage range of 8-128×10⁶ DC per patient is used. Peripheral blood monocytes are divided into 2 aliquots and pulsed with plu-1 and KLH between days 1 and 10. Serum-free culture conditions or autologous plasma is used in preference to allogeneic AB serum. Cultured DCs are pooled, washed and resuspended in 100 mls saline prior to infusion over 1 hour. The autologous red cell concentrate is not returned to the patient other than for a standard clinical indication. The ex vivo DC culture procedures are carried out following good manufacturing practices.

Patients who donated the initial blood samples will, by this time, have received salvage chemotherapy and may or may not be in clinical remission. Further patients with relapsed metastatic disease receive treatment prior to receiving chemotherapy. There are two treatment regimes:

(1) metastatic relapse, standard therapy followed by adoptive immunotherapy;

(2) metastatic relapse, adoptive immunotherapy followed by standard therapy.

Criteria to include patients for treatment are:

Patients with localised relapse or metastatic breast carcinoma.

Previous treatment with cytotoxic chemotherapy or hormonal therapy.

Evaluable disease (UICC criteria).

Survival predicted to be>12 weeks.

Fulfil criteria for autologous blood donation (including HgB>120 g/l).

Informed consent.

Age between 18 years and 70 years.

Criteria to exclude patients from treatment are:

Pregnancy.

CNS metastases.

Previous or concomitant metastases.

Unable to give informed consent.

Consent refused.

Age <18 years or >70 years.

Product infusion is carried out under the direct supervision of an experienced physician on a ward on day bed unit where resuscitation and supportive care facilities are available if required.

### EXAMPLE 4

### Polynucleotide Anti-Tumour Immunization to plu-1 Antigen in Patients with Breast Cancer

The polynucleotide anti-tumor immunization strategy employs the direct, intramuscular injection of naked plasmid DNA. The cDNA for human plu-1 is inserted into a simplified eukaryotic expression vector which utilizes separate CMV intermediate early promoter/enhancers to regulate transcription of plu-1. The plasmids are derived from the commercially available eukaryotic expression vector pcDNA3 (Invitrogen). The plasmid structure contains the cytomegalovirus early promoter/enhancer and the bovine growth hormone polyadenylation signal flanking a polylinker for insertion of heterologous open reading frames. The pcDNA3 plasmid has been modified by removal of sequences encoding the SV40 origin of replication and the neomycin resistance gene. Additionally, gene sequences encoding kanamycin resistance have been added. The plasmid DNAs are grown in the E. coli host strain DH10B. Purification is by anion exchange, ion paired reverse phase and hydrophobic interaction chromatography. Endotoxin is removed by a combination of the column chromatography and extraction with NP40. The identity of the plasmid is verified by restriction endonuclease analysis. Purity of prepared DNA is validated by gel analysis, assessment of supercoiled and linear DNA content, and residual protein content. Endotoxin and bioburden tests are also performed. A bioassay is also performed to verify expression of the plasmid-encoded cDNAs. Vialed plasmid DNA for polynucleotide immunization will be formulated in a citrate buffered saline solution containing 0.25% bupivacaine-HCl at a DNA concentration of 0.5 mg/mL.

Immunization Strategy and Schedule

Overall Approach

Patients receive progressively increasing-intensity of immunization. The decision to progress to the next dose level for a patient receiving a single vaccination will be based on lack of acute toxicity following four weeks of follow-up; ten weeks of follow-up is required for a patient receiving three immunizations.

Treatment Schedule

The following schedules may be performed:

1) 0.05 mg plu-1 polynucleotide injection into each deltoid muscle on Day 1.

2) 0.15 mg plu-1 polynucleotide injection into each deltoid muscle on Day 1.

3) 0.5 mg plu-1 polynucleotide injection into each deltoid muscle on Day 1.

4) 0.15 mg plu-1 polynucleotide injection into each deltoid muscle on Days 1, 22 and 43.

5) 0.5 mg plu-1 polynucleotide injection into each deltoid muscle on Days 1, 22 and 43.

Specific Therapeutic Plan

Each patient receives bilateral intramuscular (deltoid muscle) injections of the plu-1 polynucleotide reagent The use of bilaterial injections for each immunization is to reduce the likelihood that a technical failure of delivery into the body of the muscle will occur since such a delivery would preclude gene expression and immune response. Secondly, gene expression has been reported to be greater if more than one site is used.

The intramuscular injection technique utilizes standard aseptic technique utilizing a 1 ml syringe, 25 g needle and a volume of ≦1 ml for each injection. The patient is monitored (vital signs Q 15 minutes times 4 and Q hour times 3) for four hours for local pain, discomfort or signs of inflammation and be re-examined 24 hours later to monitor for any local or systemic signs of inflammation or toxicity. The patient is monitored by phone conversations at 48 and 72 hours and return for visits/exams weekly times 2 for evaluation for toxicity and blood samples.

Humoral and cellular immunity to plu-1 is detected as described (with reference to CEA as the antigen) in Conry et al (1996) Hum. Gene Ther. 7, 755-772; this paper describes lymphoblastic transformation assays, lymphokine release assays, CTL response assays, and serologic assays.

### EXAMPLE 5

### Recombinant plu-1 Vaccinia Virus Vaccine with Post Vaccination plu-1 Peptide Challenge

Vaccinia Virus Vaccine—Clinical Formulation and Drug Supply

The recombinant product is a frozen preparation of live vaccinia virus prepared by standard procedures and will be given at a dose of 2.5×10⁶ PFU/vaccination. The vaccine is prepared from standard strains of vaccinia virus. It has been genetically engineered using a pT108 plasmid vector to contain a copy of the human plu-1 gene in the viral genome inserted in the viral 30K gene (Hind III M fragment). Virus for vaccination is grown in CV1 monkey cell line. Each vial contains 0.1 ml (100 microliters) of bulk vaccine containing approximately 2×10⁹ plaque forming units (PFU)/ml.

Stability: The vaccine must be stored frozen at −70° C. or colder. Once thawed, the vaccine may be stored refrigerated at 2-8° for four days.

Clinical preparation: The dilutions are prepared in a laboratory laminar air flow hood by the investigator or by his assistant, or the pharmacy. 2.5×10⁶ PFU are made by first removing 19.9 ml from the saline vial with sterile technique and sterile syringe and placing this in a sterile empty vial. 100 microliters are then removed from the vaccine vial and added to the 19.9 ml of saline. A tuberculin syringe is then used to delivery approximately 2.5×10⁶ PFU/2.5 microliters volume intradermally.

Plu-1 Peptide/Detox—Clinical Formulation and Drug Supply

Peptide synthesis and verification is done using standard protocols for clinical use. Peptide used in this study is a 9-mer which starts at position 711 in the plu-1 polypeptide sequence residue GMP grade >95% pure. Residual solvent levels by gas chromatography-mass spectrometry are at acceptable levels. CAP-1 peptide is formulated as a lyophilized powder dissolved in 100% DMSO at a concentration of 3.3 mg/ml. The peptide is provided in 2 ml vials, with a total volume of 0.6 ml/vial of peptide solution and will be stored at −70° C.

Detox™ Adjuvant is formulated as a lyophilized oil droplet emulsion. Each vial, which has a red label to distinguish it from a previous formulation, contains 280 μg Cell Wall Skeleton (CWS) from Mycobacterium phlei, 28 μg of Menophosphoryl Lipid A (MPL) from Salmonella minnesota Re595, 4.5 mg squalene, 1.1 mg Tween 80, and 4.8 mg NaCl. Vials are stored at refrigerated temperature (2-8° C.).

Each vial of Detox is reconstituted with 1.4 ml of Sterile Water for Injection, USP. When 1.25 ml of the resultant emulsion is withdrawn, it contains 250 μg Cell Wall Skeleton (CWS) and 2.5 μg of MPL.

To reconstitute Detox:

1. Inject 1.4 ml of Sterile Water for Injection into the vial using a 3 cc syringe and a 22 gauge needle. Inject and aspirate repeatedly for two minutes.

2. Warm the vial of Detox in hot tap water for one to two minutes and repeat the aspiration step. Do NOT heat over 37 degrees C.

3. If the emulsion stands for any length of time, it should be shaken vigorously immediately before use.

The peptide solution is mixed with 1.45 ml of reconstituted Detox for a final volume of 2.0 ml to be delivered as follows:

The plu-1 peptide+Detox™ admixture will be administered to patients subcutaneously (sc) with 1 25-gauge, ⅝ inch needle.

Peptide vaccination is administered to the patient using any of the following doses:

1) 100 μg/2.0 ml sc

2) 500 μg/2.0 ml sc

3) 1000 μg/2.0 ml sc

4) 1500 pg/2.0 ml sc

The range of peptide dose levels is based on concentration of plu-1 peptide used in vitro for stimulation of plu-1-specific T-cells. No further group will be added because of solubility limitations (maximum 1 mg of peptide/1 ml of solution) and no intrapatient dose escalation is planned.

All patients receive 2.0 ml of peptide vaccination solution consisting of the 1.4 ml of the diluent adjuvant Detox™ admixed under sterile conditions with the appropriate dose of plu-1 peptide as follows:

1) 30 μl peptide+570 μl sterile H₂O+1.4 ml Detox

2) 150 μl peptide+450 μl sterile H₂O+1.4 ml Detox

3) 300 μl peptide+300 μl sterile H₂O+1.4 ml Detox

4) 450 μl peptide+150 μl sterile H₂O+1.4 ml Detox, corresponding to those above.

The peptide vaccine is administered subcutaneously. Each patient receives the total dose administered over the deltoids, the thighs, and the abdomen (2.0 ml/site).

Treatment Plan

Patients receive rV plu-1 and plu-1 peptide vaccination and weekly follow-up. The patients are typed as HLA-A2.

Live, recombinant vaccinia virus is thawed prior to use. A tuberculin syringe is then used to administer 250 μl (2.5×10⁶ pfu) intradermally over the deltoid muscle of either arm, thigh, or abdomen. The skin area with at least a 5 cm radius must be healthy and without infection or trauma. The site is covered by a sterile non adherent (Telfa) pad and then by a clear semipermeable (Opsite) dressing. Patients receive an instruction sheet regarding dressing care, bathing, etc.

Two vaccinations of 2.5×10⁶ PFU rV plu-1 are administered to each patient at four week intervals unless there is unacceptable toxicity or the patient is unable to receive the treatment as the result of debilitating disease progression.

Subsequent to the rV plu-1 vaccinations, three vaccinations with plu-1 peptide Detox adjuvant will be administered at four week intervals unless there is unacceptable toxicity or the patient is unable to receive the treatment as the result of debilitating disease progression. Dose escalations will proceed on the following schedule:

1) 100 μg/2.0 ml sc

2) 500 μg/20 ml sc

3) 1000 μg/2.0 ml sc

4) 1500 μg/2.0 ml sc

### EXAMPLE 6

### In situ Hybridisation on plu-1 mRNA

In situ hybridisation was performed with the plu-1 probe 253g2. This probe contains nucleotides 3633-5559 of the plu-1 cDNA (ie mainly 3′ untranslated region, UTR). The hybridisation was carried out essentially as described in Senior et al (1988) Development 104, 431-446.

There is an increased expression of plu-1 mRNA in progression from benign to ductal carcinoma in situ (DCIS) to invasive tumour epithelium. Cysts and lactating epithelium are generally weak. However, in sample 45-96C (human breast grade 1 ductal tumour; FIG. 15(a)) an increase in plu-1 mRNA is shown in the invasive tissue.

In sample 19996G (human breast grade 3 ductal tumour; FIG. 15(b)) the presence of low level plu-1 mRNA is seen in a cyst, increased levels of plu-1 mRNA are in a DCIS region, and further increased levels of plu-1 mRNA are seen in invasive tissue.

Thus, for breast tumour samples, plu-1 mRNA is absent/weak in benign breast tumours, there is some expression in DCIS (an early stage of carcinogenesis), and increased plu-1 expression in invasive breast carcinomas.

In a small non-breast survey, prostate epithelial cells were weakly positive, foetal spermatic cords were positive and abnormal adult testis gave signals in sertoli cells. In 14.8 week foetal tissues, a subset of foetal kidney tubule epithelium and some urothelium was positive; nerve ganglia next to the spinal cord and liver were also positive for c112 mRNA. Heart appeared negative but other foetal muscles were questionably positive.

1. A recombinant polynucleotide encoding a polypeptide comprising the plu-1 amino acid sequence shown in FIG. 2 or variants or fragments or derivatives or fusions thereof or fusions of said variants or fragments or derivatives.

2. A polynucleotide according to claim 1 which contains no introns.

3. A polynucleotide according to claims 1 or 2 comprising the polynucleotide whose sequence is shown in FIG. 1.

4. A polynucleotide according to any one of the preceding claims, comprising the polynucleotide whose sequence is shown in FIG. 1 between positions 90 and 4724.

5. A replicable vector comprising a polynucleotide as defined in any one of claims 1 to 4.

6. A host cell comprising a recombinant polynucleotide or a replicable vector as defined in any one of claims 1 to 5.

7. A host cell comprising a recombinant polynucleotide or a replicable vector as defined in any one of claims 1 to 5 wherein the host cell is not a bacterium.

8. A host cell according to claim 7 wherein the host cell is an animal cell.

9. A host cell according to claim 8 wherein the host cell is a mammalian cell.

10. A method of making a polypeptide having the amino acid sequence shown in FIG. 2 or variants or fragments or fusions or derivatives thereof, or fusions of said variants or fragments or derivatives, the method comprising culturing a host cell as defined in any one of claims 6 to 9 which expresses said variant or fragment or derivative or fusion and lo isolating said polypeptide or variant or fragment or derivative or fusion from said host cell culture.

11. A polypeptide comprising the amino acid sequence shown in FIG. 2 or variants or fragments or fusions or derivatives thereof or fusions of said variants or fragments or derivatives.

12. A polypeptide obtainable by the method of claim 10.

13. An antibody reactive towards the polypeptide whose amino acid sequence is shown in FIG. 2 or natural variants thereof.

14. An antibody according to claim 13 which is not substantially reactive towards any other polypeptide.

15. An antibody reactive towards an epitope present in the-polypeptide whose amino acid sequence is shown in FIG. 2 or natural variants thereof.

16. An antibody according to claim 15 wherein the epitope is not present in any other polypeptide.

17. An antibody according to claims 13 or 14, reactive towards a molecule comprising any one of the peptides QQTDRSSPVRPSSEKNDC, PKDMNNFKLERERSYELVR or CTVKDAPSRK.

18. A method of making an antibody which is reactive towards the polypeptide whose amino acid sequence is shown in FIG. 2 or a natural variant thereof, the method comprising the steps of, where appropriate, immunising an animal with a plu-1 peptide and selecting an antibody which binds plu-1.

19. A method according to claim 18 wherein the peptide distinguishes plu-1 from any other polypeptide and the antibody does not substantially bind any other polypeptide.

20. A molecule which, following immunisation of an animal if appropriate, gives rise to antibodies which are reactive towards the polypeptide whose sequence is shown in FIG. 2 or natural variants thereof.

21. A molecule according to claim 20 wherein said antibodies are not reactive towards any other polypeptide.

22. A molecule according to claim 20 or 21 which is a peptide comprising any one of the sequences QQTDRSSPVRPSSEKNDC, PKDMNNFKLERERSYELVR or CTVKDAPSRK.

23. A polynucleotide which distinguishes a polynucleotide which encodes the polypeptide whose sequence is shown in FIG. 2 or a natural variant thereof and a polynucleotide which encodes any other polypeptide.

24. A polynucleotide which hybridises to a polynucleotide which encodes the polypeptide whose sequence is shown in FIG. 2 or a natural variant thereof but not to a polynucleotide which encodes any other polypeptide.

25. A polynucleotide according to claims 23 or 24, wherein the polynucleotide is an oligonucleotide.

26. A polynucleotide according to any one of claims 23 to 25, wherein the polynucleotide which encodes the polynucleotide whose sequence is shown in FIG. 2 or a natural variant thereof or the polynucleotide which encodes the other polypeptide is a MRNA or a cDNA.

27. A method for determining the susceptibility of a patient to cancer comprising the steps of
(i) obtaining a sample containing nucleic acid from the patient; and
(ii) contacting the said nucleic acid with a nucleic acid which hybridises selectively to plu-1 nucleic acid.

28. A method of diagnosing cancer in a patient comprising the steps of
(i) obtaining a sample containing nucleic acid from the patient; and
(ii) contacting the said nucleic acid with a nucleic acid which hybridises selectively to plu-1 nucleic acid.

29. A method of predicting the relative prospects of a particular outcome of a cancer in a patient comprising the steps of
(i) obtaining a sample containing nucleic acid from the patient; and
(ii) contacting the said nucleic acid with a nucleic acid which hybridises selectively to plu-i nucleic acid.

30. A method according to any one of claims 27 to 29 wherein the plu-1 nucleic acid is mRNA.

31. A method according to any one of claims 27 to 29 wherein the plu-1 nucleic acid is DNA and its methylation status is determined.

32. A method according to any one of claims 27 to 31 wherein the cancer is ovarian cancer or breast cancer.

33. A method according to any one of claims 27 to 32 wherein the sample is a sample of the tissue in which cancer is suspected or in which cancer may be or has been found.

34. A method according to any one of claims 27 to 33 wherein the sample is a sample of breast and the cancer is breast cancer.

35. A method according to any one of claims 27 to 34 wherein the nucleic acid which selectively hybridises to the plu-1 nucleic acid, further comprises a detectable label.

36. A method according to any one of claims 27 to 35 wherein the nucleic acid which selectively hybridises as said is single-stranded.

37. A method for determining the susceptibility of a patient to cancer comprising the steps of
(i) obtaining a sample containing protein derived from the patient; and
(ii) determining the relative amount, or intracellular location, of the plu-1 polypeptide.

38. A method of diagnosing cancer in a patient comprising the steps of
(i) obtaining a sample containing protein derived from the patient; and
(ii) determining the relative amount, or intracellular location, of the plu-1 polypeptide.

39. A method of predicting the relative prospects of a particular outcome of a cancer in a patient comprising the steps of
(i) obtaining a sample containing protein derived from the patient; and
(ii) determining the relative amount, or intracellular location, of the plu-1 polypeptide.

40. A method according to any one of claims 37 to 39 wherein the cancer is ovarian cancer or breast cancer.

41. A method according to any one of claims 37 to 40 wherein the sample is a sample of the tissue in which cancer is suspected or in which cancer may be or has been found.

42. A method according to any one of claims 37 to 41 wherein the sample is a sample of breast and the cancer is breast cancer.

43. A method according to any one of claims 37 to 42 wherein the relative amount of the plu1 polypeptide is determined using a molecule which selectively binds to plu-1 polypeptide or a natural variant or fragment thereof.

44. A method according to claim 43 wherein the molecule which selectively binds plu-1 polypeptide or a natural variant or fragment thereof is an anti-plu-1 antibody.

45. A method according to claim 43 or claim 44 wherein the molecule which selectively binds to plu-1 comprises a detectable label.

46. A method of detecting a cancer in a patient the method comprising administering to the patient an anti-plu-1 antibody or a fragment or derivative thereof labelled with a detectable label, allowing the labelled antibody to locate to the cancer, and imaging the cancer.

47. Use of a nucleic acid which selectively hybridises to plu-1 MRNA in the manufacture of a reagent for diagnosing cancer.

48. Use of a molecule which selectively binds to plu-1 polypeptide or a natural fragment or variant thereof in the manufacture of a reagent for diagnosing or imaging cancer.

49. Use of a nucleic acid as defined in claim 47 in a method of diagnosing cancer.

50. Use of a molecule which selectively binds to plu-1 polypeptide or a natural fragment or variant thereof in a method of diagnosing or imaging cancer.

51. A method of treating cancer comprising the step of administering to the patient a nucleic acid which encodes the plu-i polypeptide or a functional variant or portion or fusion thereof.

52. Use of plu-1 polypeptide or a variant or fragment thereof, or a nucleic acid which encodes the plu-1 polypeptide or a functional variant or portion or fusion thereof in the manufacture of a medicament for treating cancer.

53. A method of treating cancer, the method comprising administering to the patient an effective amount of plu-I polypeptide or a variant or fusion or fragment thereof, or an effective amount of a nucleic acid encoding a plu-1 polypeptide or a variant or fragment or fusion thereof, wherein the amount of said polypeptide or amount of said nucleic acid is effective to provoke an anti-cancer cell immune response in said patient.

54. A cancer vaccine comprising plu-1 polypeptide or variant or fragment thereof, or a nucleic acid encoding plu-1 polypeptide or fragment or variant thereof.

55. A method for producing activated cytotoxic T lymphocytes (CTL) in vitro, the method comprising contacting in vitro CTL with antigen-loaded human class I MHC molecules expressed on the surface of a suitable cell for a period of time sufficient to activate, in an antigen specific manner, said CTL wherein the antigen is an antigenic peptide derived from the plu-1 polypeptide.

56. A method of specifically killing target cells in a human patient which target cells express the plu-1 polypeptide, the method comprising (1) obtaining a sample containing precursor CTL from said patient, (2) contacting, in vitro, said CTL with antigen-loaded human class I MHC molecules expressed on the surface of a suitable cell for a period of time sufficient to activate, in an antigen specific manner, said CTL wherein the antigen is an antigenic peptide derived from the plu-1 polypeptide.

57. A method of treating a patient with cancer, the method comprising obtaining dendritic cells from said patient, contacting said dendritic cells with an antigenic peptide derived from the plu-1 polypeptide, or with a polynucleotide encoding said antigenic peptide, ex vivo, and reintroducing the so treated dendritic cells into the patient.

58. A method of treating a patient with cancer the method comprising administering to the patient an effective amount of a plu-1 antisense agent.

59. Use of plu-1 polypeptide or an active variant or fragment or derivative or fusion thereof or an active fusion of a variant or fragment or derivative thereof in an assay for identifying compounds which modulate the activity of the plu-1 polypeptide.

60. Use of an antibody which selectively binds plu-1 or a fragment or derivative thereof for treating, diagnosing or imaging cancer.

61. A polypeptide according to claim 11 for use in medicine.

62. An antibody according to claim 14 or 15 for use in medicine.

63. A nucleic acid which hybridises selectively to plu-1 nucleic acid for use in medicine.

64. A kit of parts comprising an antibody according to claim 14 or 15 and a control sample comprising plu-1 polypeptide or an immunoreactive fragment thereof.

65. A kit of parts comprising a nucleic acid which hybridises selectively to plu-1 nucleic acid and a control sample comprising a plu-1 nucleic acid.

66. A kit of parts according to claim 64 further comprising components for testing for a further cancer-related polypeptide.

67. A kit of parts according to claim 65 further comprising a nucleic acid which selectively hybridises to a further cancer-related nucleic acid.

68. A pharmaceutical composition comprising plu-1 polypeptide or a variant or fragment or derivative or fusion thereof or a fusion of a variant or fragment or derivative thereof and a pharmaceutically acceptable carrier.

69. A pharmaceutical composition comprising a nucleic acid encoding plu-1 polypeptide or a variant or fragment or derivatives or fusion thereof or a fusion of a variant or fragment or derivative thereof and a pharmaceutically acceptable carrier.
